(19) United States
(12) Patent Application Publication
(10) Pub. No.: US 2026/0207796 A1
(43) Pub. Date: Jul. 23, 2026

(54) METHODS AND MATERIALS FOR COMBINING BIOLOGICS WITH MULTIPLE CHELATORS

(71) Applicant: Mayo Foundation for Medical Education and Research, Rochester, MN (US)

(72) Inventors: Mukesh K. Pandey, Rochester, MN (US); Aditya Bansal, Rochester, MN (US); Geoffrey B. Johnson, Rochester, MN (US); David J. Bartlett, Rochester, MN (US)

(21) Appl. No.: 19/137,834

(22) PCT Filed: Dec. 21, 2023
(86) PCT No.: PCT/US2023/085553

§ 371 Date: Jun. 11, 2025

Related U.S. Application Data
(60) Provisional application No. 63/434,451, filed on Dec. 21, 2022.

(51) Int. Cl. A61K 51/04; A61K 51/12

(52) U.S. Cl. CPC A61K 51/0497; A61K 51/044; A61K 51/1203; A61K 2121/00

(57) ABSTRACT

Conjugates that include two or more chelators including functional groups capable of stabilizing a radiometal (e.g., a chelator of a radiotherapy isotope and a chelator of an imaging isotope) covalently attached to one or more binding moieties are provided herein. The conjugates can be used for treating cancer or non-cancer conditions, and can serve as both an imaging and a radiotherapy molecule when the imaging isotope is complexed to the chelator of the imaging isotope and the radiotherapy isotope is complexed to the chelator of the radiotherapy isotope.

### CROSS-REFERENCE TO RELATED APPLICATIONS

This application claims the benefit of U.S. Provisional Application Ser. No. 63/434,451, filed Dec. 21, 2022. The disclosure of the prior application is considered part of the disclosure of this application, and is incorporated in its entirety into this application.

### BACKGROUND

### 1. Technical Field

This document relates to conjugates of two or more chelators (e.g., a conjugate of a chelator of an isotope for imaging and a chelator of an isotope for radiotherapy) and one or more binding moieties, and using such conjugates for treating diseases such as cancer. For example, this document provides methods and materials for combining a binding moiety with two or more chelators, wherein one of the chelators is a chelator of an isotope used for imaging and one of the chelators is a chelator of an isotope used for radiotherapy. A conjugate in which the imaging isotope and the radiotherapy isotope are complexed to the chelators can be administered to a mammal in need of treatment, and can serve as both an imaging and a radiotherapy molecule.

### 2. Background Information

In the field of targeted radionuclide therapy, the ability to accurately calculate dosimetry (how much therapy drug has gone to tumors and tissues in the body) through imaging of a patient is a powerful way to understand the disease pathology, disease progression, and response to radionuclide therapy, and also helps to enhance drug development via a better understanding of pharmacokinetic and pharmacodynamics, expediting regulatory (e.g., FDA) approvals and personalize care for patients (e.g., cancer patients). The field of targeted radionuclide therapy is moving toward more effective and often more expensive alpha-emitters, and away from beta-emitters. However, alpha-emitters are typically not suited for imaging due to the unavailability or low abundance of the appropriate positron or photon-energy emissions (511 KeV for PET and 100-200 KeV for SPECT). The high linear energy transfer (LET) of alpha-emission, and the γ-photons, characteristic x-rays, or bremsstrahlung radiation that accompany decay of the parent alpha emitting radionuclide are poorly suited for quantifying target uptake, dosimetry, and therapy response compared to beta-emitters. Furthermore, even when performing therapy with beta-emitters that can be imaged, the beta-emitters are often imaged poorly with SPECT technology. If imaging of radionuclide therapies could be performed with PET technology, the resolution, accuracy, and quality of the images would be superior. As a result, most research and development, FDA submissions, and clinical programs have to depend on estimated biodistribution/dosimetry based on poor quality images or by using a surrogate imaging probe (a modified drug that can be imaged). These surrogate imaging probes differ significantly from the alpha-emitter therapy drug in multiple ways, making them less optimal for predicting the biodistribution/dosimetry of the alpha-emitting therapy drug. Therefore, there is a need for improved radiotherapies that can be imaged directly and accurately.

### SUMMARY

This document is based, at least in part, on the discovery of a method of combining (e.g., covalently attaching) a binding moiety or motif, e.g., a biologic or drug that binds to a target molecule in a mammal, with multiple chelators such that the resulting conjugate or mixtures of conjugates can serve simultaneously as both an imaging and radiotherapy molecule when suitable isotopes are complexed with the chelators. The resulting conjugates include two or more chelators and a binding moiety (e.g., two or more chelators covalently attached to a binding moiety via one or more linkers), wherein one of the chelators is a chelator of an isotope used for imaging (referred to herein as a “chelator of an imaging isotope”) and one of the chelators is a chelator of an isotope used for radiotherapy (referred to herein as a “chelator of a radiotherapy isotope”). As described herein, the conjugates can be selectively used for imaging or radionuclide therapy as needed by choosing radionuclides for imaging or therapy and filling the other chelator with a non-radioactive version of the imaging or therapy metal ion to maintain the same chemical nature of the molecule. Using the same chemical entity preserves the same biodistribution, and avoids using surrogate imaging probes that differ in structure and can have a different biodistribution. In addition, the same conjugate can be used for both imaging and radionuclide therapy by complexing both the chelators with appropriate imaging and therapy radionuclides, without being forced to choose only a single isotope that is suboptimal at one or both tasks. This disclosure is based, at least in part, on a realization that the stability of a radiometal within a chelator moiety may be enhanced by a functional group or groups located on the chelator moiety. As described more fully herein, suitable examples of stability-enhancing functional groups include amine, hydroxy, amide, carboxy, and other anionic and cationic functional groups, capable, e.g., to form a ligand for the radiometal.

The conjugates and methods described herein can allow the biodistribution and dosimetry of alpha-emitting therapy drugs to be evaluated prior to therapy and also evaluated with each cycle of radiotherapy, helping to speedup research and development, speedup FDA approvals, and guide clinical care. In addition, the methods described herein can be used to streamline the ongoing evaluation of patients who are receiving these expensive radiotherapies with more accurate therapy monitoring (e.g., by imaging of the therapy right after it is administered) and can do so with a straightforward clinical workflow. This can result in informed changes in the care-plan mid therapy, saving money by stopping futile therapy early, improving outcomes by adjusting or augmenting therapy when needed, or switching to a more effective therapy sooner.

The conjugates described herein can be designed so the half-life of the imaging isotope (e.g., an isotope for positron emission tomography (PET) or an isotope for single photon emission computed tomography (SPECT)) and the physical half-life of the radiotherapy isotope (e.g., an alpha or beta emitting radionuclide) are matched to ensure that the biodistribution of the therapy over the time it is radioactive can be imaged and therefore dosimetry can be accurately calculated. For example, the half-life of the imaging isotope (e.g., an isotope for PET or an isotope for SPECT), the physical half-life of the radiotherapy isotope (e.g., an alpha or beta emitting radionuclide), and the plasma half-life of a targeting vector (e.g., peptide, antibody, or small molecule) can be matched to ensure that the biodistribution of the therapy over the time it is radioactive can be imaged and dosimetry can be accurately calculated. In some embodiments, an optical imaging (near infra-red) probe can be added to the conjugate. The conjugates and methods described herein provide a robust platform to stage the disease, treat the disease, monitor the response to therapy or progression, and/or minimize side effects to healthy organs and tissues, all with versions of the same molecule (chemically and biologically identical). This can be achieved by simply choosing whether a conjugate described herein is complexed with an isotope for imaging and/or complexed with an isotope for radiotherapy or non-radioactive versions of these same isotopes (i.e., radionuclides can be swapped with non-radioactive isotopes that have different nuclear structures but are chemically identical) for the desired use of the conjugate. In some embodiments, two or more conjugates can be used. For example, in some embodiments, one conjugate described herein is complexed with an alpha-emitting isotope for therapy and one conjugate described herein is complexed with a positron-emitting isotope for imaging. Additionally, the conjugates described herein can include more than one binding moiety or motif to enhance the uptake in the targeted tissues/organs.

In one general aspect, this document provides a conjugate comprising two or more chelators and a binding moiety (e.g., a targeting vector). In some embodiments, one of said chelators is a chelator of an imaging isotope and one of said chelators is a chelator of a radiotherapy isotope.

In some embodiments, the present disclosure provides a conjugate comprising two or more chelators and a binding moiety, wherein one of said chelators is a chelator of an imaging isotope and one of said chelators is a chelator of a radiotherapy isotope,
wherein at least one chelator within the conjugate comprises one or more functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, —C(═S)NH₂, and C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
provided that the functional groups in said at least one chelator are not all C(═O)NH₂ or C(═O)OH;
and wherein said chelators and said binding moiety are linked via a moiety of Formula (I):

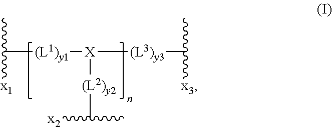

wherein:
   each X is independently selected from N, P, P(═O), CR^{N}, and a moiety of formula (i):

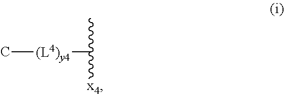

each of x₁, x₂, x₃, and x₄ independently indicates a point of attachment of the moiety of Formula (I) to said chelators or said binding moiety;
each of L¹, L², L³, and L⁴ is independently selected from C(═O), C(═S), N(R^{N}), O, S, S(═O), S(═O)₂, —CR^{N}=NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
each of y₁, y₂, y₃, and y₄ is independently an integer from 1 to 10;
each R^{N} is independently selected from H, C₁₋₃ alkyl, and C₁₋₃ haloalkyl; and
n is an integer selected from 1, 2, 3, 4, and 5.

In some embodiments, said compound of Formula (I) has formula:

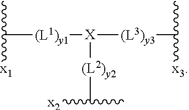

In some embodiments, the moiety of Formula (I) has any one of the following formulae:

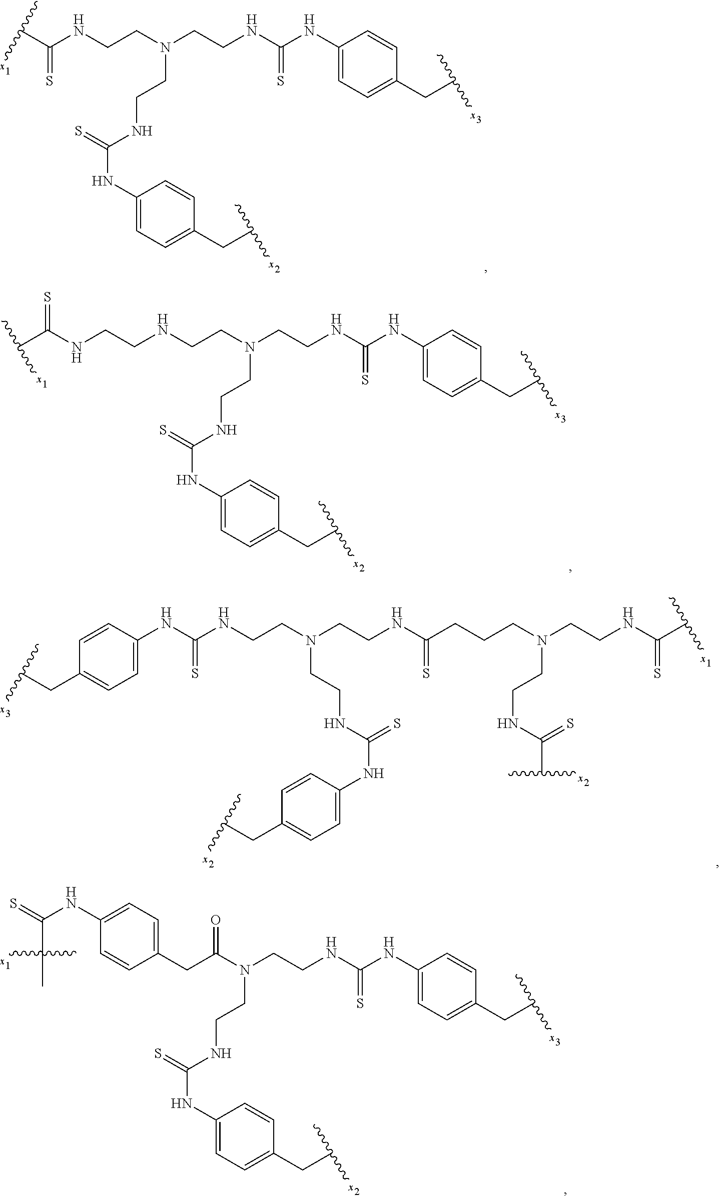 [SMILES: *Cc1ccc(NC(=S)NCCN(CCNC(*)=S)CCNC(=S)Nc2ccc(C*)cc2)cc1 ; *Cc1ccc(NC(=S)NCCN(CCNCCNC(*)=S)CCNC(=S)Nc2ccc(C*)cc2)cc1 ; *Cc1ccc(NC(=S)NCCN(CCNC(=S)CCCN(CCNC(*)=S)CCNC(*)=S)CCNC(=S)Nc2ccc(C*)cc2)cc1 ; *Cc1ccc(NC(=S)NCCN(CCNC(=S)Nc2ccc(C*)cc2)C(=O)Cc2ccc(NC(*)=S)cc2)cc1]

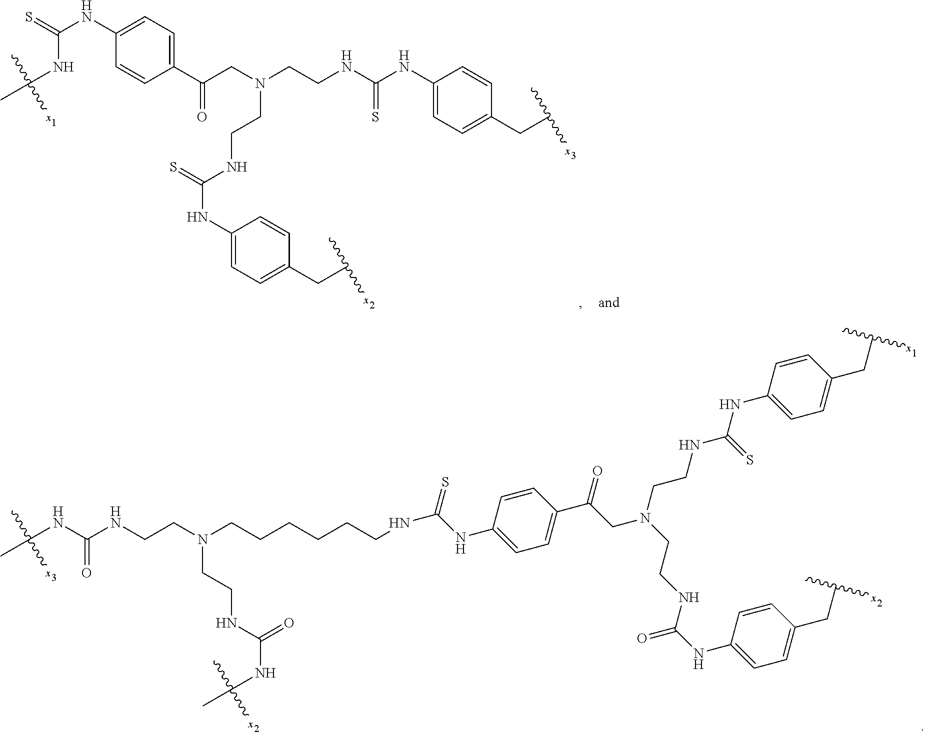 [SMILES: *Cc1ccc(NC(=S)NCCN(CCNC(=S)Nc2ccc(C*)cc2)CC(=O)c2ccc(NC(=S)N*)cc2)cc1 ; *Cc1ccc(NC(=O)NCCN(CCNC(=S)Nc2ccc(C*)cc2)CC(=O)c2ccc(NC(=S)NCCCCCCN(CCNC(=O)N*)CCNC(=O)N*)cc2)cc1]

In some embodiments, chelator of an imaging isotope independently comprises a compound selected from the group consisting of 1,4,7-triazacyclononane-1,4,7-triacetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA), each of which comprises one or more functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, —C(═S)NH₂, and C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments, chelator of an imaging isotope is substituted with 3 or 4 functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, provided that at least one functional group is other than C(═O)NH₂ or C(═O)OH.

In some embodiments, chelator of an imaging isotope has formula:

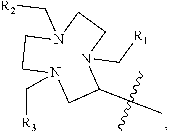

wherein each R¹, R², and R³ is independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and
[symbol] indicates a point of attachment to Formula (I).

In some embodiments, at least one of R¹, R², and R³ is other than C(═O)NH₂ or C(═O)OH.

In some embodiments, said imaging isotope is ^{133/135}La, ¹³³Ce, ⁶⁸Ga, ⁴⁴Sc, ^{60/61/62/64}Cu, ^{84/86/87/89}Zr, ⁶³Zn, ^{43/44}Sc, ^{192/193/194/196}Au, ^{52m}Mn, ^{90,92m1}Nb, ^{51/52}Mn, ^{148/151/151m/152}Tb, ⁴⁵Ti, ^{65/66/67}Ga, ^{94m}Tc, ⁵⁵Co, ^{80/81/83}Sr, ³⁸K, ^{70/71/72/74}As, ^{81/82m}Rb, ⁵²Fe, or ⁸⁶Y.

In some embodiments, said imaging isotope is ⁶⁴Cu.

In some embodiments, chelator of a radiotherapy isotope independently comprises a compound selected from the group consisting of 1,4,7-triazacyclononane-1,4,7-triacetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA), each of which comprises one or more functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, —C(═S)NH₂, and C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments, chelator of a radiotherapy isotope is substituted with 3 or 4 functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, provided that at least one functional group is other than C(═O)NH₂ or C(═O)OH.

In some embodiments, chelator of a radiotherapy isotope has formula:

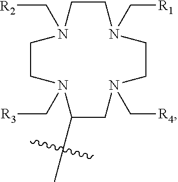

wherein each R¹, R², R³, and R⁴ is independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and
[symbol] indicates a point of attachment to Formula (I).

In some embodiments, at least one of R¹, R², R³, and R⁴ is other than C(═O)NH₂ or C(═O)OH.

In some embodiments, said radiotherapy isotope is an α-emitter.

In some embodiments, said radiotherapy isotope is ^{223/225/227}Ac, ²¹²Pb, ²¹¹At, ²¹³Bi, ²¹²Bi, ²¹¹Bi, ^{152/160/161}Tb, ²²⁷Th, ²²³Ra, ²¹¹Po, ²²¹Fr, ²¹⁷At, ²¹³Po, ²¹²Po, ²¹⁵Po, or ¹⁷⁷Lu.

In some embodiments, said radiotherapy isotope is ²¹²Pb.

In some embodiments, the conjugate is selected from any one of the following formulae:

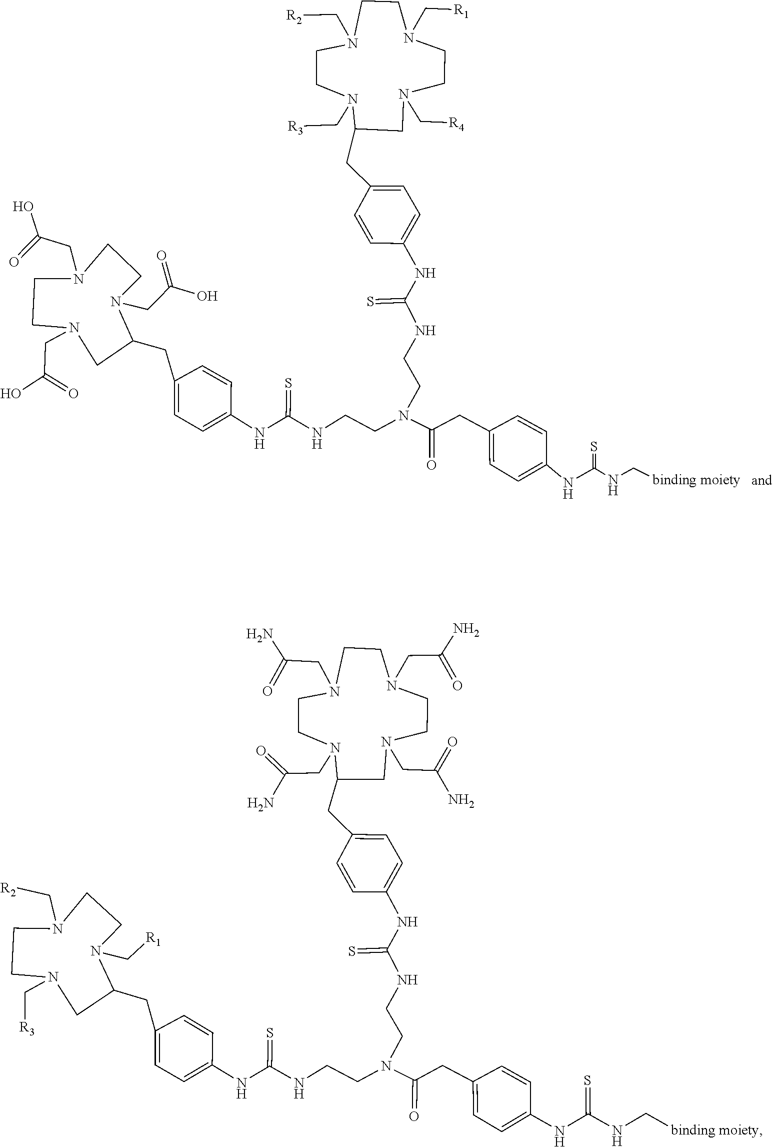

wherein each R¹, R², R³, and R⁴ in any of the above formulae is independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and at least one of R¹, R², R³, and R⁴ is other than C(═O)NH₂ or C(═O)OH.

In some embodiments, the binding moiety is selected from a polypeptide, an antibody, a small molecule, an extracellular vesicle, a virus, and a nucleic acid.

In some embodiments, said polypeptide binds prostate specific membrane antigen, a somatostatin receptor, or a melanocortin-1 receptor.

In some embodiments, said small molecule is a glutamate carboxypeptidase II inhibitor.

In some embodiments, said small molecule is a glucose or a derivative thereof.

In some embodiments, the conjugate is selected from any one of the following compounds:

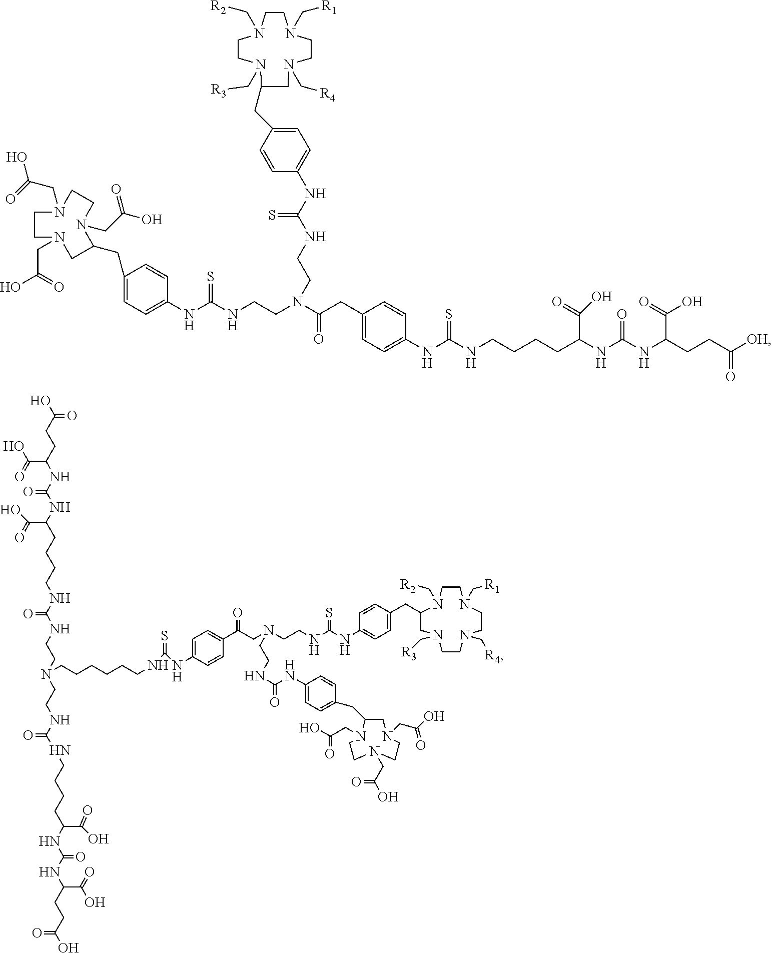

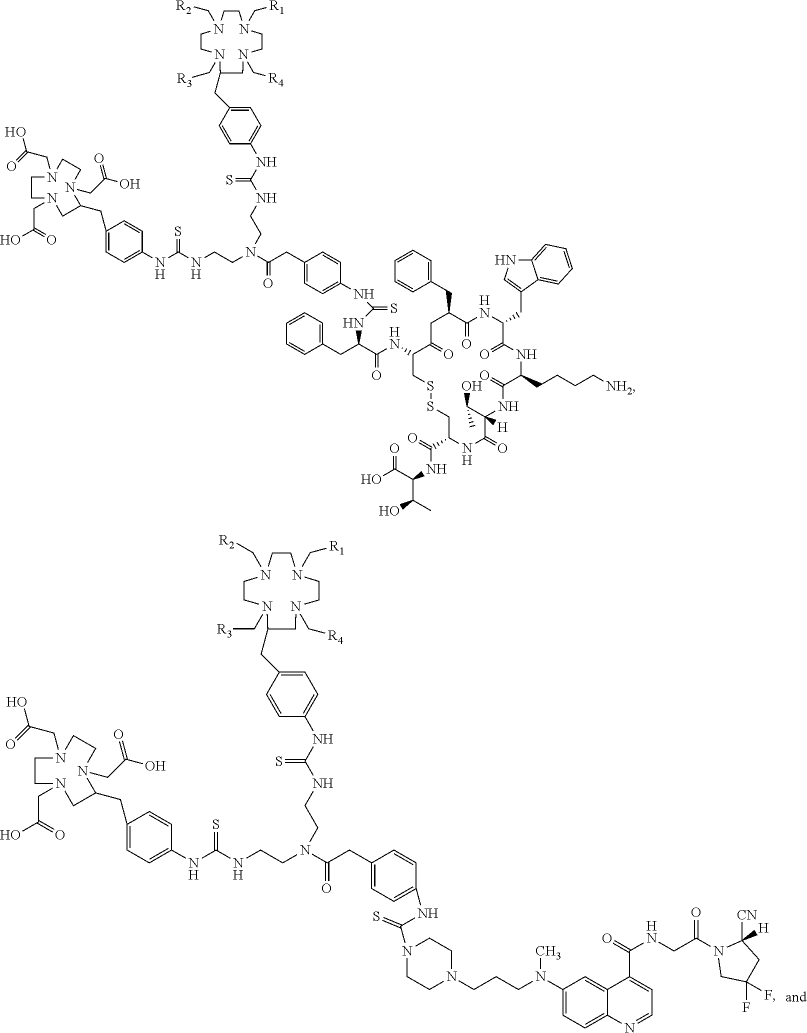

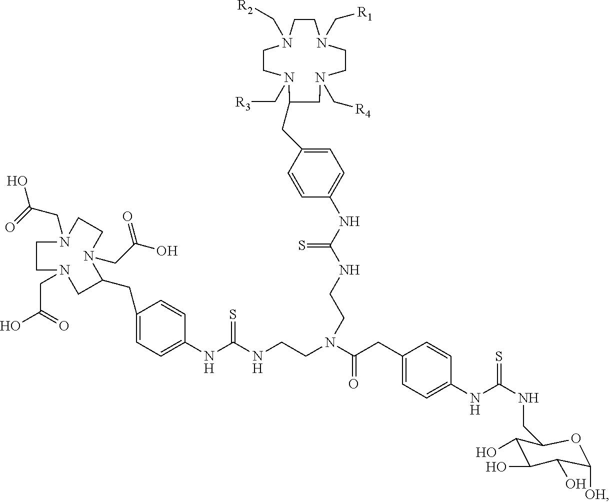

wherein each R¹, R², R³, and R⁴ in any of the above formulae is independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and at least one of R¹, R², R³, and R⁴ is other than C(═O)NH₂ or C(═O)OH.

In some embodiments, the present disclosure provides a conjugate comprising two or more chelators and a binding moiety, wherein one of said chelators is a chelator of an imaging isotope and one of said chelators is a chelator of a radiotherapy isotope,
wherein the binding moiety is selected from an extracellular vesicle, a virus, a nucleic acid, and a glucose, or a derivative thereof,
and wherein said chelators and said binding moiety are linked via a moiety of Formula (I):

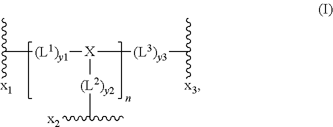

wherein:
   each X is independently selected from N, P, P(═O), CR^{N}, and a moiety of formula (i):

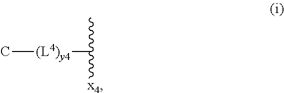

each of x₁, x₂, x₃, and x₄ independently indicates a point of attachment of the moiety of Formula (I) to said chelators or said binding moiety;
each of L¹, L², L³, and L⁴ is independently selected from C(═O), C(═S), N(R^{N}), O, S, S(═O), S(═O)₂, —CR^{N}=NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
each of y₁, y₂, y₃, and y₄ is independently an integer from 1 to 10;
each R^{N} is independently selected from H, C₁₋₃ alkyl, and C₁₋₃ haloalkyl; and
n is an integer selected from 1, 2, 3, 4, and 5.

In some embodiments, the binding moiety is an extracellular vesicle.

In some embodiments, the binding moiety is a virus.

In some embodiments, the binding moiety is a nucleic acid.

In some embodiments, said compound of Formula (I) has formula:

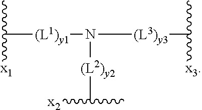

In some embodiments, the moiety of Formula (I) has any one of the following formulae:

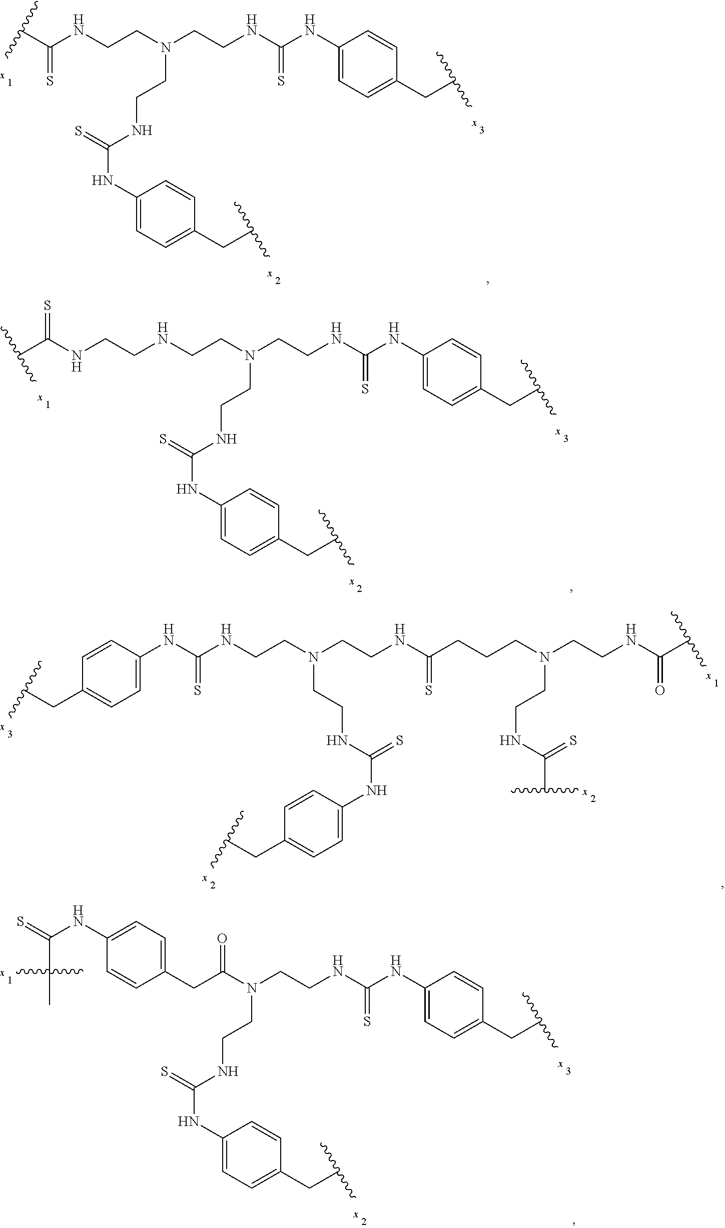

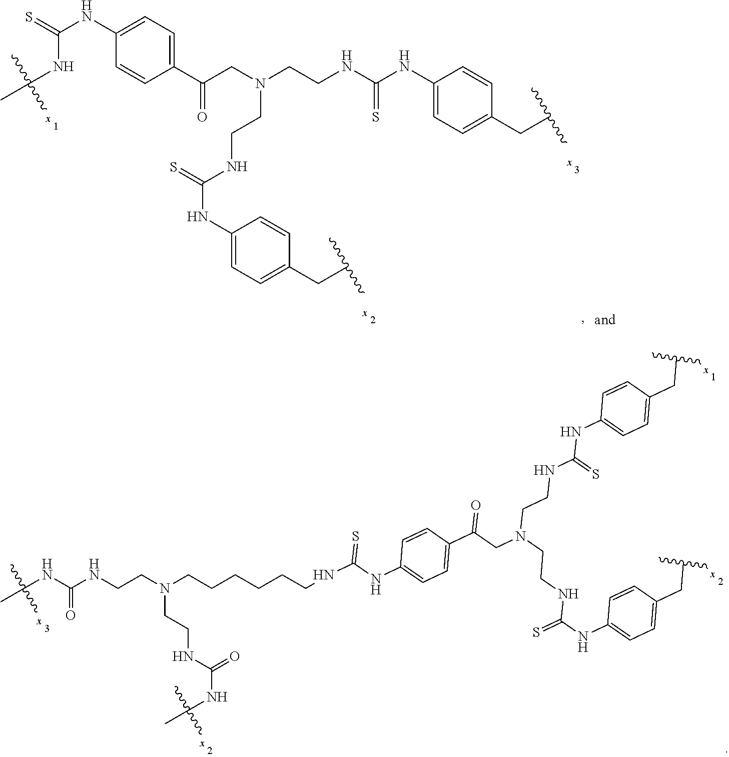

In some embodiments, chelator of an imaging isotope independently comprises a compound selected from the group consisting of 1,4,7-triazacyclononane-1,4,7-triacetic acid (NOTA), 2,2′,2″,2′″-(1,4,7,10-Tetraazacyclododecane-1,4,7,10-tetrayl)tetraacetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA).

In some embodiments, chelator of a radiotherapy isotope independently comprises a compound selected from the group consisting of 1,4,7-triazacyclononane-1,4,7-triacetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA).

In some embodiments, chelator of an imaging isotope is 1,4,7-triazacyclononane-1,4,7-triacetic acid (NOTA) and chelator of a radiotherapy isotope is 1,4,7,10-tetrakis(carbamoylmethyl)-1,4,7,10-tetracyclododecane (TCMC).

In some embodiments, said imaging isotope is ^{133/135}La, ¹³³Ce, ⁶⁸Ga, ⁴⁴Sc, ^{60/61/62/64}Cu, ^{84/86/87/89}Zr, ⁶³Zn, ^{43/44}Sc, ^{192/193/194/196}Au, ^{52m}Mn, ^{90/92m1}Nb, ^{51/52}Mn, ^{148/151/151m/152}Tb, ⁴⁵Ti, ^{65/66/67}Ga, ^{94m}Tc, ⁵⁵Co, ^{80/81/83}Sr, ³⁸K, ^{70/71/72/74}As, ^{81/82m}Rb, ⁵²Fe, or ⁸⁶Y.

In some embodiments, said imaging isotope is ⁶⁴Cu.

In some embodiments, said radiotherapy isotope is an α-emitter.

In some embodiments, said radiotherapy isotope is ^{223/225/227}Ac, ²¹²Pb, ²¹¹At, ²¹³Bi, ²¹²Bi, ²¹¹Bi, ^{152/160/161}Tb, ²²⁷Th, ²²³Ra, ²¹¹Po, ²²¹Fr, ²¹⁷At, ²¹³Po, ²¹²Po, ²¹⁵Po, or ¹⁷⁷Lu.

In some embodiments, said radiotherapy isotope ²¹²Pb.

In some embodiments, the conjugate is selected from any one of the following compounds:

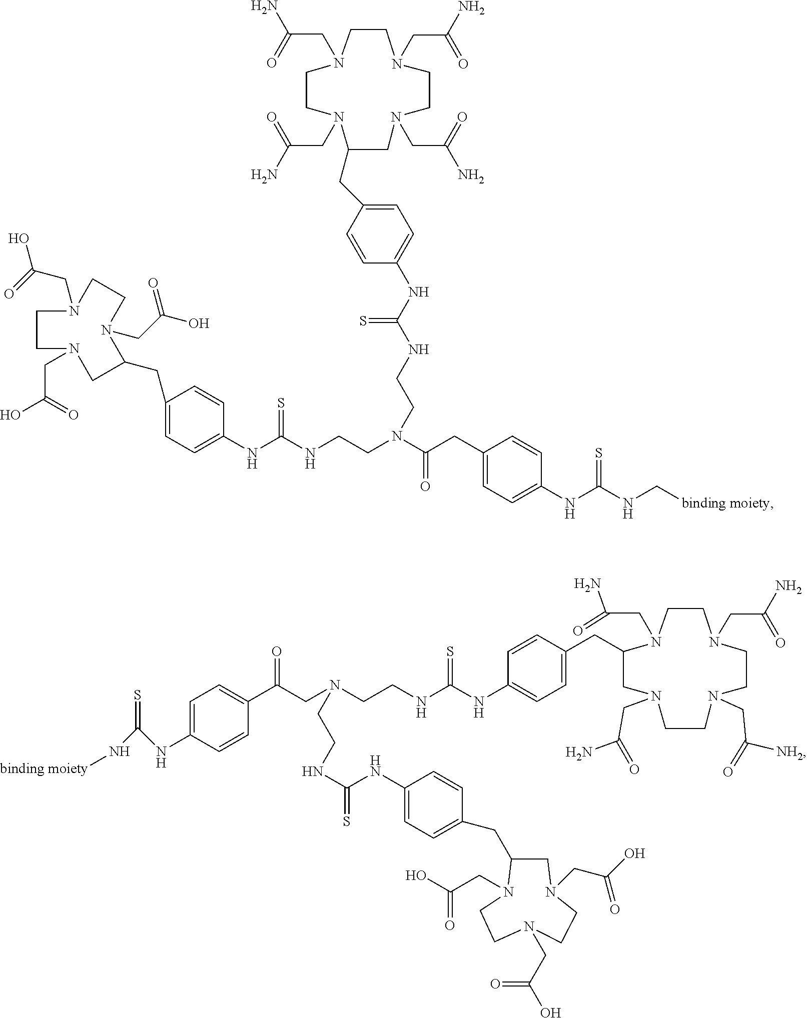

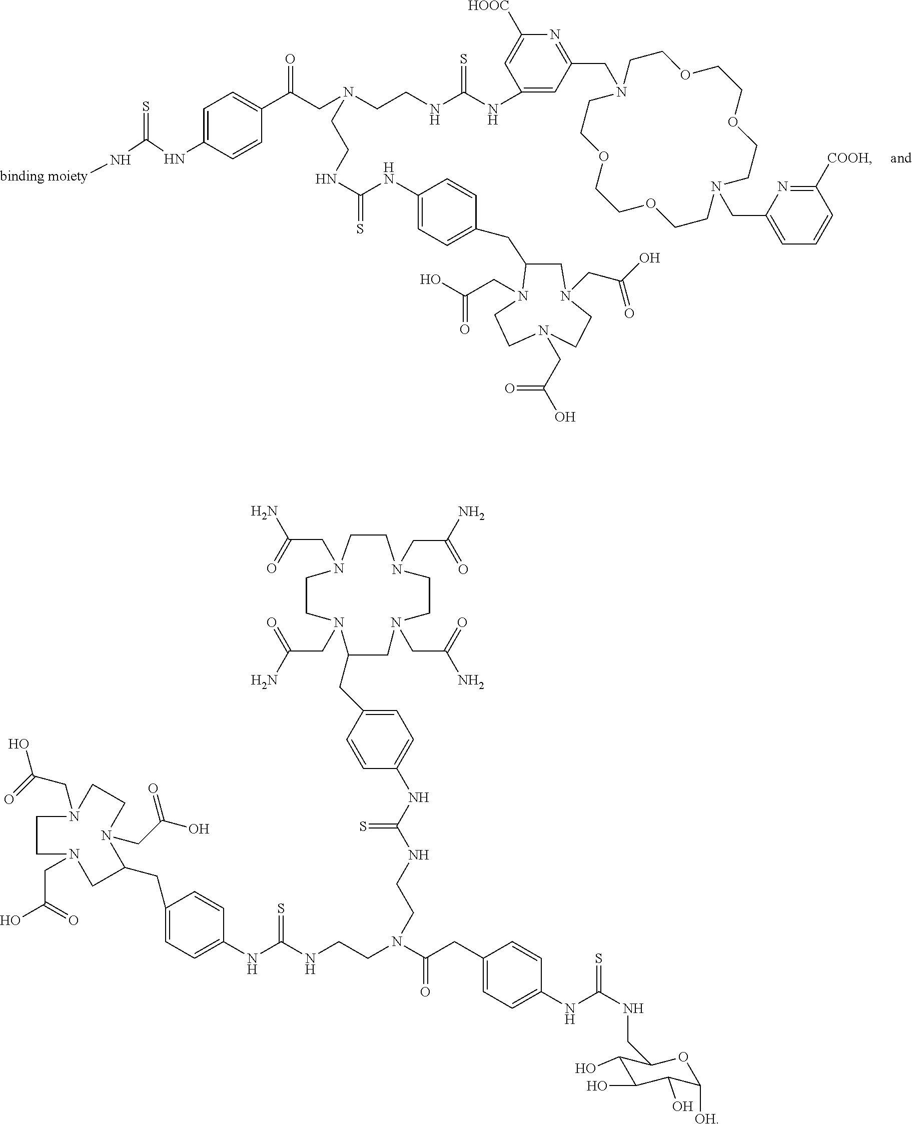

In some aspects of the above embodiments, the binding moiety is selected from an extracellular vesicle, a virus, a nucleic acid, and a glucose, or a derivative thereof.

In some embodiments, the present disclosure provides a conjugate as described herein, wherein the conjugate comprises an imaging isotope complexed to the chelator of the imaging isotope and/or wherein the conjugate comprises a radiotherapy isotope complexed to the chelator of the radiotherapy isotope.

In some embodiments, the present disclosure provides method of treating cancer in a mammal in need thereof, wherein said method comprises administering a conjugate as described herein to said mammal, wherein said conjugate comprises said imaging isotope complexed to said chelator of said imaging isotope and wherein said conjugate comprises said radiotherapy isotope complexed to said chelator of said radiotherapy isotope.

In some embodiments, the present disclosure provides a method of treating cancer in a mammal, wherein said method comprises:
a) administering, to said mammal, a first conjugate as described herein comprising two or more chelators and a binding moiety, wherein one of said chelators is a chelator of an imaging isotope and one of said chelators is a chelator of a radiotherapy isotope, wherein said first conjugate comprises said imaging isotope complexed to said chelator of said imaging isotope;
b) determining, in said mammal, the biodistribution of said first conjugate; and
c) administering, to said mammal, an amount of a second conjugate as described herein that is identical to said first conjugate except that said second conjugate comprises said radiotherapy isotope complexed to said chelator of said radiotherapy isotope.

In some embodiments, the method further comprises determining, in said mammal, the biodistribution of said second conjugate comprising said imaging isotope complexed to said chelator of said imaging isotope and said radiotherapy isotope complexed to said chelator of said radiotherapy isotope.

In some embodiments, said cancer is selected from the group consisting of prostate cancer, a neuroendocrine cancer, colon cancer, breast cancer, fibroblast associated protein cancers, brain cancer, liver cancer, ovarian cancer, thyroid cancers, lung cancer, pancreatic cancer, melanoma, and a lymphoid cancer.

In some embodiments, the cancer is a metastasized tumor.

In some embodiments, the present disclosure provides a method of treating cancer in a mammal in need thereof, wherein said method comprises administering, to said mammal, two or more conjugates as described herein,
wherein each conjugate comprises two or more chelators and a binding moiety, wherein one of said chelators is a chelator of an imaging isotope and one of said chelators is a chelator of a radiotherapy isotope,
wherein one of said conjugates administered to said mammal comprises an imaging isotope complexed to said chelator of said imaging isotope, and
wherein one of said conjugates administered to said mammal comprises a radiotherapy isotope complexed to said chelator of said radiotherapy isotype.

In some embodiments, the binding moiety is glucose, or a derivative thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a decay scheme of ²¹²Pb.

FIGS. 2A and 2B are examples of conjugates of two or more chelators linked to a binding moiety.

FIG. 3A contains chemical structure of imaging drug within the present claims (radiopharmaceutical containing imageable Cu isotope).

FIG. 3B contains chemical structure of radionuclide therapy drug (radiopharmaceutical). The compounds in FIGS. 3A and 3B represent a concept entailing both single and or dual injection of imaging and radionuclide therapy drugs as per need. An imaging drug could be separately synthesized with imaging isotope (e.g. Cu-64) and loaded with nonradioactive version of therapy isotope (e.g., Pb) or vice versa. These drug could be injected to humans as a single injection of imaging or therapy drug or dual/mixed injection of both therapy and imaging drug post independent synthesis, formulation and characterization.

FIG. 4A contains chemical structure of Pb conjugate with functional groups designed to promote Pb stability. Examples of R¹, R², R³, and R⁴ include NH₂, OH, and NHOH. COR¹, COR², COR³, and COR⁴ can be any of —SO₃H, —SO₂NH₂, —PO₄H₂, —PO₃HNH₂—CONH₂, —COOH, —CONHOH, —CS₂H, —COSH, —CSOH, and —CSNH₂. Pb could be any isotopes of Pb including but not limited to ²¹²Pb, ²⁰³Pb, and Pb; and Cu could be any isotope of Cu including but not limited to ⁶⁴Cu, ⁶⁷Cu, and ⁶¹Cu. The targeting vector could be a small molecule, peptide, protein or antibody or other biologics including but not limited to extracellular vesicles, viruses, nucleotides and nucleosides, RNA, or DNA. Targeting vector could be targeting any disease or medical condition.

FIG. 4B contains chemical structure of ⁶⁴Cu conjugate with functional groups designed to promote its stability. Examples of R¹, R², and R³ include NH₂, OH, and NHOH. COR¹, COR², and COR³ can be any of —SO₃H, —SO₂NH₂, —PO₄H₂, —PO₃HNH₂—CONH₂, —COOH, —CONHOH, —CS₂H, —COSH, —CSOH, and —CSNH₂. Pb could be any isotopes of Pb including but not limited ²¹²Pb, ²⁰³Pb, and Pb; and Cu could be any isotope of Cu including but not limited to ⁶⁴Cu, ⁶⁷Cu, and ⁶¹Cu. The targeting vector could be a small molecule, peptide, protein or antibody or other biologics including but not limited to extracellular vesicles, viruses, nucleotides and nucleosides, RNA, or DNA. Targeting vector could be targeting any disease or medical condition.

FIG. 5 contains chemical structures of conjugates with representative examples of binding moieties (e.g., targeting vectors such as glucose or a derivative thereof).

FIG. 6A contains a scheme showing synthesis of a conjugate to treat small metastasized tumors with Alpha-PET technology. The depicted conjugates complexed to radioisotopes specially designed to target small, metastasized tumors with glucose analog. In this design, glucose and glucose analogs are acting as targeting vectors, and the alpha-PET platform molecule after conjugation facilitates its imaging and radionuclide therapy.

FIG. 6B contains a scheme showing conjugation of Alpha-PET platform moiety with extracellular vesicle and radiolabeling.

FIG. 7 contains a scheme showing conjugation of Alpha-PET platform moiety with different types of viruses and radiolabeling.

FIG. 8 contains a scheme showing conjugation of Alpha-PET platform moiety with different aptamers and radiolabeling.

FIG. 9 shows a structure of [⁶⁴Cu]Cu-NOTA-TCMC-Adeno-Associated Virus ([⁶⁴Cu]Cu-NOTA-TCMC-AAV).

FIG. 10 shows a radio-TLC chromatogram showing A. ⁶⁴Cu and B. [⁶⁴Cu]Cu-NOTA-TCMC-AAV9 with 100 mM Sodium Citrate (pH 5.0) as mobile phase.

FIG. 11 shows a representative maximum intensity projection (MIP)-PET/X-Ray atlas image showing biodistribution of [⁶⁴Cu]Cu-NOTA-TCMC-AAV9 (~2.0 μCi) in BALB/c mouse at different time points post-injection. Li=Liver; SUV=Standardized Uptake Value.

FIG. 12 shows a chemical structure of [⁶⁴Cu]Cu-NOTA-TCMC-Extracellular Vesicle ([⁶⁴Cu]Cu-NOTA-TCMC-EV).

FIG. 13 shows a radio-TLC chromatogram showing A. ⁶⁴Cu and B. [⁶⁴Cu]Cu-NOTA-TCMC-EVs with 100 mM Sodium Citrate (pH 5.0) as mobile phase.

FIG. 14 shows a representative maximum intensity projection (MIP)-PET/X-Ray atlas image showing biodistribution of [⁶⁴Cu]Cu-NOTA-TCMC-EVs (~23.25 μCi) in BALB/c mouse at different time points post-injection. Li=Liver, Gu=Gut; SUV=Standardized Uptake Value.

### DETAILED DESCRIPTION

This document provides conjugates that include two or more chelators and one or more binding moieties or motifs, wherein one of the chelators is a chelator of an imaging isotope and one of the chelators is a chelator of a radiotherapy isotope. A trifunctional compound (e.g., such as N′,N′-bis(2-aminoethyl)ethane-1,2-diamine), which can act as a linker, can be selectively reacted with two different chelators, one for an imaging isotope and one for a radiotherapy isotope, to produce a dual chelator compound. The dual chelator compound can be modified to make it suitable to react with the binding moiety (e.g., modified at room temperature under mild reaction condition (such as an aqueous medium) to protect the nature and functionality of the binding moieties, to produce a conjugate in which the two or more chelators are covalently attached to the one or more binding moieties or motifs. Only one functional group on the targeted binding moiety (e.g., a primary NH₂) is needed to produce the conjugate. As described below, the combination of chelators and isotopes can be varied as needed for the method of treatment or imaging.

In some embodiments, the chelators can be linked to the binding moiety with a moiety of Formula (I):

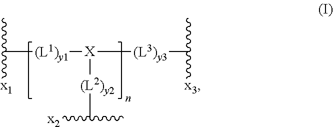

wherein:
each X is independently selected from N, P, P(═O), CR^{N}, and a moiety of formula (i):

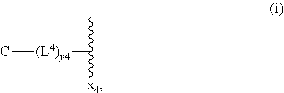

each of x₁, x₂, x₃, and x₄ independently indicates a point of attachment of the moiety of Formula (I) to a chelator or a binding moiety;
each of L¹, L², L³, and L⁴ is independently selected from C(═O), C(═S), N(R^{N}), O, S, S(═O), S(═O)₂, —CR^{N}=NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
each of y1, y₂, y₃, and y₄ is independently an integer from 1 to 10;
each R^{N} is independently selected from H, C₁₋₃ alkyl, and C₁₋₃ haloalkyl; and
n is an integer selected from 1, 2, 3, 4, and 5.

In some embodiments, X is N.

In some embodiments, X is P.

In some embodiments, X is P(═O).

In some embodiments, X is CR^{N}.

In some embodiments, X is the moiety of formula (i).

In some embodiments, X is selected from N and CR^{N}.

In some embodiments, X is selected from N, CR^{N}, and the moiety of formula (i).

In some embodiments, each L¹ independently selected from C(═O), C(═S), NH, O, —C₁₋₃ alkylene-, and C₆₋₁₀ arylene. In some embodiments, moiety (L¹)y₁ comprises at least one moiety of formula NHC(═S)NH or C₆₋₁₀ arylene-C₁₋₃ alkylene-.

In some embodiments, each L² independently selected from C(═O), C(═S), NH, O, —C₁₋₃ alkylene-, and C₆₋₁₀ arylene. In some embodiments, moiety (L²)y₂ comprises at least one moiety of formula NHC(═S)NH or C₆₋₁₀ arylene-C₁₋₃ alkylene-.

In some embodiments, each L³ independently selected from C(═O), C(═S), NH, O, —C₁₋₃ alkylene-, and C₆₋₁₀ arylene. In some embodiments, moiety (L³)y₃ comprises at least one moiety of formula NHC(═S)NH or C₆₋₁₀ arylene-C₁₋₃ alkylene-.

In some embodiments, each L⁴ independently selected from C(═O), C(═S), NH, O, —C₁₋₃ alkylene-, and C₆₋₁₀ arylene. In some embodiments, moiety (L⁴)y₄ comprises at least one moiety of formula NHC(═S)NH or C₆₋₁₀ arylene-C₁₋₃ alkylene-.

In some embodiments, y₁ is an integer selected from 1, 2, 3, 4, 5, and 6. In some embodiments, y₂ is an integer selected from 1, 2, 3, 4, 5, and 6. In some embodiments, y₃ is an integer selected from 1, 2, 3, 4, 5, and 6. In some embodiments, y₄ is an integer selected from 1, 2, 3, 4, 5, and 6.

In some embodiments, R^{N} is H. In some embodiments, R^{N} is C₁₋₃ alkyl. In some embodiments, R^{N} is selected from H and C₁₋₃ alkyl.

In some embodiments, n is 1. In some embodiments, n is 2. In some embodiments, n is 3. In some embodiments, n is 4.

In some embodiments, the compound of Formula (I) has formula:

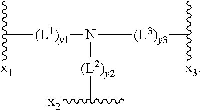

In some embodiments, the compound of Formula (I) has formula:

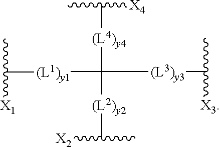

In some embodiments, the compound of Formula (I) has formula:

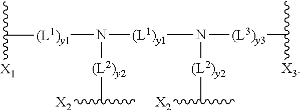

In some embodiments, the moiety of Formula (I) can have any one of the following formulae:

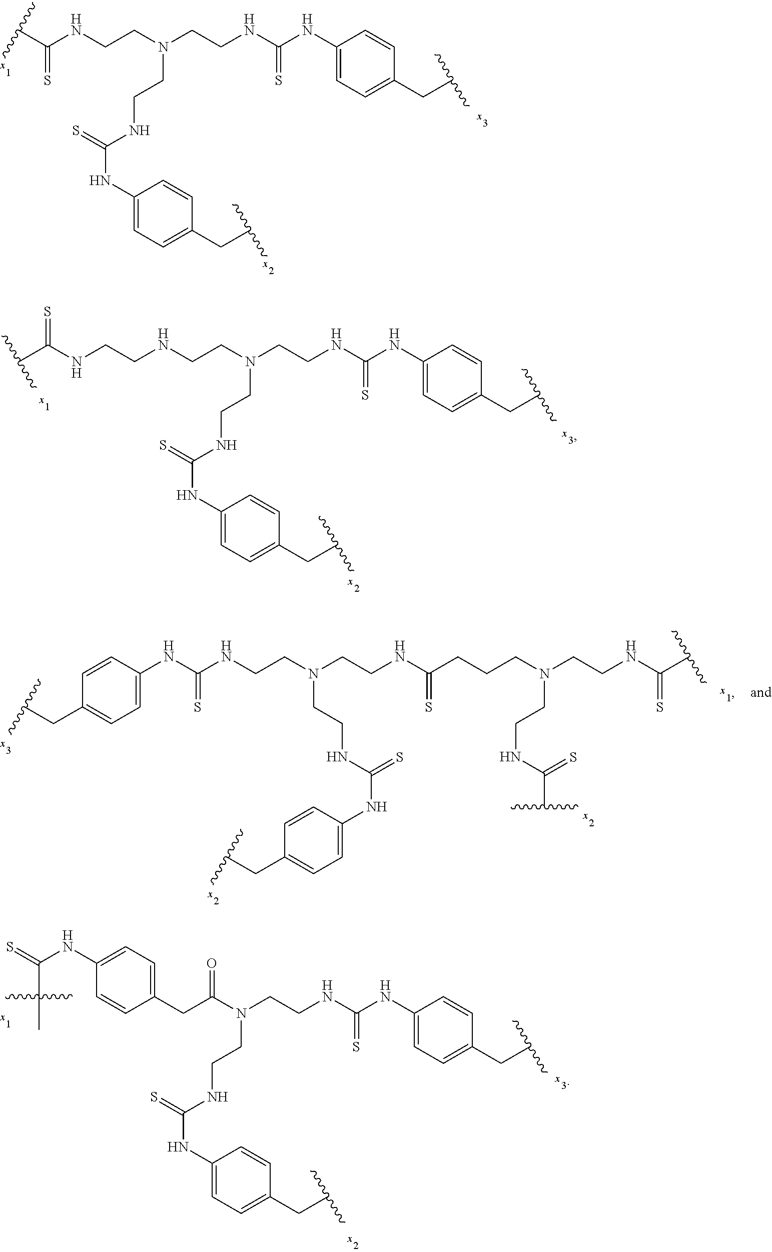

In some embodiments, the chelators are linked and/or the chelator and the binding moiety are linked with a moiety of Formula (II):

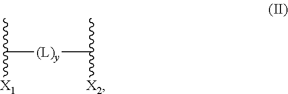

wherein:
x₁ indicates a point of attachment of the Formula (II) to the chelator;
x₂ indicates a point of attachment of the Formula (II) to the chelator or the binding moiety;
each L is independently selected from C(═O), C(═S), N(R^{N}), O, S, S(═O), S(═O)₂, —CR^{N}=NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
y is an integer from 1 to 30; and
each R^{N} is independently selected from H, C₁₋₃ alkyl, and C₁₋₃ haloalkyl.

In some embodiments, x₂ indicates a point of attachment of the Formula (II) to the chelator. In some embodiments, x₂ indicates a point of attachment of the Formula (II) to or the binding moiety.

In some embodiments, each L independently selected from C(O), C(═S), NH, O, —C₁₋₃ alkylene-, and C₆₋₁₀ arylene. In some embodiments, moiety (L)y comprises at least one moiety of formula NHC(═S)NH or C₆₋₁₀ arylene-C₁₋₃ alkylene-.

In some embodiments, y is an integer from 1 to 10. In some embodiments, y is 1, 2, 3, 4, 5, 6, 7, 8, 9, or 10. In some embodiments, R^{N} is H. In some embodiments, R^{N} is C₁₋₃ alkyl. In some embodiments, R^{N} is selected from H and C₁₋₃ alkyl.

In some embodiments, the moiety of Formula (II) has any one of the following formulae:

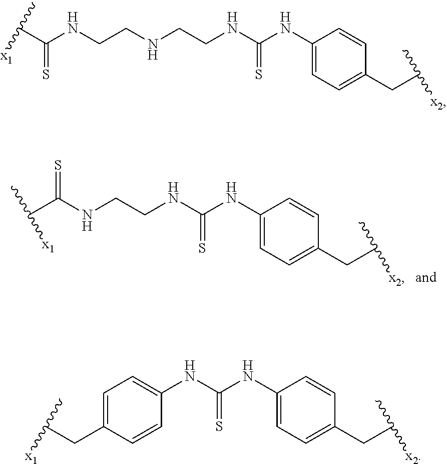

In some embodiments, the chelator can be linked to the binding moiety with a cleavable linker. As used herein, the term “cleavable linker” refers to a linker that is readily catabolized or metabolized under specific conditions. In some cases, a cleavable linker can remain intact under most conditions (e.g., while in storage) but can be cleaved when exposed to a particular compound (e.g., a compound present in the body such as a particular protease) such that the linker is cleaved when in the presence of that compound. In some cases, a cleavable linker can remain intact under most conditions (e.g., while in storage) but can be cleaved under physiological conditions (e.g., at a human's natural blood pH) such that the linker is cleaved when administered to a mammal (e.g., a human). For example, in some embodiments, the cleavable linker can be acid cleavable, GSH cleavable, Fe(II) cleavable, cathepsin cleavable, glycosidase cleavable, phosphatase cleavable, sulfatase cleavable, photo-responsive cleavable, or biorthogonal cleavable. See, for example, Zheng et al., Acta Pharm Sin B. 2021 December, 11(12):3889-3907 and Tsuchikama et al., Protein Cell. 2018 January; 9(1):33-46. In some cases, the cleavable moiety can be as described in U.S. Pat. No. 11,191,854 or 10,093,741. For example, in some embodiments, the cleavable moiety can comprise an ester bond, a phosphate bond, or a disulfide bond. An ester linkage can be cleavable by an esterase native to the cellular environment or hydrolyzable by a neutral or acidic buffered environment. A phosphate linkage can be cleavable by a phosphatase or hydrolyzable by a neutral or acidic buffered environment. A disulfide linkage can be cleavable by the reducing environment of the microenvironment, soluble GSH, thioredoxin, or glutaredoxin. Once cleavage occurs, the binding moiety can maintain its extended retention within the body while the chelators and associated radionuclei can be rapidly excreted.

In some embodiments, the cleavable linker can connect the binding moiety to one or more chelators. For example, the cleavable linker can connect the binding moiety to two chelators. In some embodiments, cleavage of the linker can separate one or more chelators from the binding moiety.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cn-m” indicates a range that includes the endpoints, wherein n and m are integers and indicate the number of carbons. Examples include C₁₋₄, C₁₋₆, and the like.

As used herein, the term “Cn-m alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cn-m haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cn-m alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cn-m alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms. In a similar manner, the term “Cₙ₋ₘ alkenylene” refers to, employed alone or in combination with other terms, refers to a divalent alkenyl linking group having n to m carbons, and the term Cₙ₋ₘ alkynyl,” employed alone or in combination with other terms, refers to a divalent alkynyl linking group having n to m carbons.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “carboxy” refers to a —C(O)OH group. As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C=N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, each of the chelators independently can be, for example, NOTA, DOTA, TCMC, DiAmSar, HBED, DFO, DTPA, H₄pypa, NTA, BisTris, EGTA, EDTA, BAPTA, DO2A, DO3A and MACROPA. In general, a combination of chelators for imaging and therapy isotopes can be selected for a particular application. For example, in some embodiments, one chelator can be DiAmSar and one chelator can be TCMC. In some embodiments, one chelator can be NOTA and one chelator can be TCMC. In some embodiments, each of the chelators independently can be a supermagnetic iron oxide nanoparticle (SPION). In some embodiments, the SPION can be ferumoxytol. Certain aspects of these embodiments are described, for example, in Advanced Drug Delivery Reviews, Volume 63, Issues 1-2, January-February 2011, Pages 24-46; and Kidney Int. 2017 July; 92(1): 47-66, which are incorporated herein by reference in their entirety.

In some embodiments, the conjugate can include three or more chelators. For example, in some embodiments, the conjugate can include three chelators, or four chelators, or five chelators. For example, in some embodiments, one chelator can be DiAmSar, one chelator can be TCMC, and one chelator can be NOTA. In some embodiments, each of the three chelators can be NOTA or each of the chelators can be SPION. In some embodiments, one chelator can be MACROPA, one chelator can be DFO, and one chelator can be DOTA. For example, in some embodiments, the conjugate can include three or more of DOTA, NOTA, TCMC, MACROPA, DiAmSar, and HBED. In some cases, one chelator can be DOTA, one chelator can be NOTA, one chelator can be TCMC, one chelator can be MACROPA, one chelator can be DiAmSar, and one chelator can be HBED.

In some embodiments, the chelator within the conjugate comprises one or more (1, 2, 3, 4, 5, or 6; or 2, 3, 4, or 5; or 3, 4, or 5; or 3 or 4) functional groups that are different from a carboxylic acid or an amide (or from corresponding hydroxyl group or an NH₂ group). In some embodiments, the chelator comprises at least one functional group selected from S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, —C(═S)NH₂, and C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl. In some embodiments, the chelator comprises at least one functional group selected from S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂.

In some embodiments, the chelator comprises at least one functional group each independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, —C(═S)NH₂, and C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl. In some embodiments, the functional groups in the chelator are not all C(═O)NH₂. In some embodiments, the functional groups in the chelator are not all C(═O)OH.

In some embodiments, the chelator within the conjugate comprises at least one S(═O)₂OH. In some embodiments, the chelator within the conjugate comprises at least one, —S(═O)₂NH₂. In some embodiments, the chelator within the conjugate comprises at least one, —OP(═O)(OH)₂. In some embodiments, the chelator within the conjugate comprises at least one —P(═O)(OH)₂. In some embodiments, the chelator within the conjugate comprises at least one-OP(═O)(OH)NH₂. In some embodiments, the chelator within the conjugate comprises at least one-C(═O)NHOH. In some embodiments, the chelator within the conjugate comprises at least one C(═S)SH. In some embodiments, the chelator within the conjugate comprises at least one C(═O)SH. In some embodiments, the chelator within the conjugate comprises at least one —C(═S)OH. In some embodiments, the chelator within the conjugate comprises at least one —C(═S)NH₂. In some embodiments, the chelator within the conjugate comprises at least one C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments, chelator (e.g., of an imaging isotope or of a radiotherapy isotope) independently comprises a compound selected from the group consisting of 1,4,7-triazacyclononane-1,4,7-tri acetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA).

In some embodiments, chelator (e.g., of an imaging isotope or of a radiotherapy isotope) independently comprises a compound selected from the group consisting of 1,4,7-triazacyclononane-1,4,7-triacetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA), each of which comprises one or more functional groups independently selected from C(═O)NH₂, C(═O)OH, —S(═O)₂OH, —S(═O)₂NI-2, —OP(═O)(OH)₂, —P(═O)(OH)₂, —OP(═O)(OH)NH₂, —C(═O)NHOH, —C(═S)SH, —C(═O)SH, —C(═S)OH, —C(═S)NH₂, and C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments, chelator (e.g., of an imaging isotope or of a radiotherapy isotope) is substituted with 3 or 4 functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, provided that at least one functional group is other than C(═O)NH₂ or C(═O)OH.

In some embodiments, chelator (e.g., of an imaging isotope or of a radiotherapy isotope) has formula:

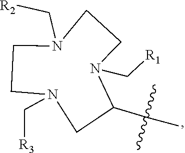

wherein each R¹, R², and R³ is independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and
[symbol] indicates a point of attachment to Formula (I).

In some embodiments, at least one of R¹, R², and R³ is other than C(═O)NH₂ or C(═O)OH.

In some embodiments, chelator (e.g., of an imaging isotope or of a radiotherapy isotope) has formula:

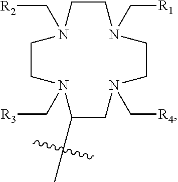

wherein each R¹, R², R³, and R⁴ is independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and
[symbol] indicates a point of attachment to Formula (I).

In some embodiments, at least one of R¹, R², R³, and R⁴ is other than C(═O)NH₂ or C(═O)OH.

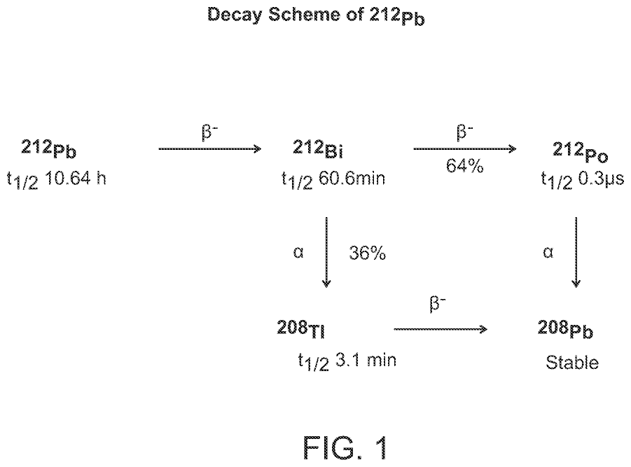

The imaging isotope and the radiotherapy isotope of a conjugate described herein can be selected such that the half-lives are similar. For example, the radiotherapy isotope can be an α-emitter such as ^{223/225/227}Ac, ²¹²Pb, ²¹¹At, ²¹³Bi, ²¹²Bi, ²¹¹Bi, ^{152/160/161}Tb, ²²⁷Th, ²²³Ra, ²¹¹Po, ²²¹Fr, ²¹⁷At, ²¹³Po, ²¹²Po, ²¹⁵Po, or ¹⁷⁷Lu and the imaging isotope can be ^{133/135}La, ¹³³Ce, ⁶⁸Ga, ⁴⁴Sc, ^{60/61/62/64}Cu, ^{84/86/87/89}Zr, ⁶³Zn, ^{43/44}Sc, ^{192/193/194/196}Au, ^{52m}Mn, ^{90/92m1}Nb, ^{51/52}Mn, ^{148/151/151m/152}Tb, ⁴⁵Ti, ^{65/66/67}Ga, ^{94m}Tc, ⁵⁵Co, ^{80/81/83}Sr, ³⁸K, ^{70/71/72/74}As, ^{81/82m}Rb, ⁵²Fe, or, ⁸⁶Y. In some embodiments, the imaging isotope is ⁶⁴Cu and the radiotherapy isotope is ²¹²Pb. ⁶⁴Cu is a positron-emitting PET imaging radionuclide, which decays to stable non-radioactive daughter nuclides ⁶⁴Ni and ⁶⁴Zn. ²¹²Pb is a parent isotope of ²¹²Bi, which is an alpha-emitting therapeutic radionuclide, which eventually decays to a stable non-radioactive daughter nuclide ²⁰⁸Pb. See, e.g., FIG. 1. ⁶⁴Cu has a physical half-life of 12.7 hours and ²¹²Pb has a physical half-life of 10.6 hours (or an effective physical half-life for alpha-emission of 11.65 hours, as described below), making them an ideal pair for evaluating the relevant radioactive biodistribution and dosimetry of ²¹²Pb using ⁶⁴Cu as the imaging readout. The longer half-lives (as compared to ⁶⁸Ga or ¹⁸F) also allow for a central location for production to cover large parts of the USA and long-distance distribution of the resulting compounds. Strictly speaking, in terms of the radioactive decay, ²¹²Pb is a beta-emitter that decays into an alpha-emitter, ²¹²Bi. ²¹²Pb is commonly referred to as an alpha-emitter among physicians because the beta-emissions that result from decay of ²¹²Pb are of little consequence physiologically relative to the alpha-emissions. Specifically, after a ²¹²Pb radionuclide gives off a beta-emission, the ²¹²Pb becomes ²¹²Bi (a daughter product) and remains in the chelator and part of the therapy drug. The ²¹²Bi then further decays by one of two equivalent pathways (see FIG. 1); (1) ²¹²Bi gives off an alpha-emission and becomes ²⁰⁸Tl, then gives off a beta-emission, or (2) ²¹²Bi gives off a beta-emission, becomes ²¹²Po and stays in the chelator, then immediately gives off an alpha-emission. Thus, ²¹²Pb and drugs containing ²¹²Pb (including those described herein) can be thought of as alpha-emitters with a physical half-life of 11.65 hours prior to alpha-emission (10.64 hours for ²¹²Pb plus 60.6 minutes for ²¹²Bi). As described herein, the decay scheme of ²¹²Pb (FIG. 1) results in 1 alpha-emission also happens to give off 2 beta-emissions as it decays to stable ²⁰⁸Pb. The beta-emissions are of no significant consequence because a beta-emission has ~10,000 times less mass than an alpha-emission and therefore the 2 beta-emission are inconsequential by comparison to the alpha-emission in terms of the effects within the body. When beta-emitters are used for therapy, the total amount of radioactive drug that needs to be injected to see an effect is orders of magnitude higher than the dose of a comparable alpha-emitting drug.

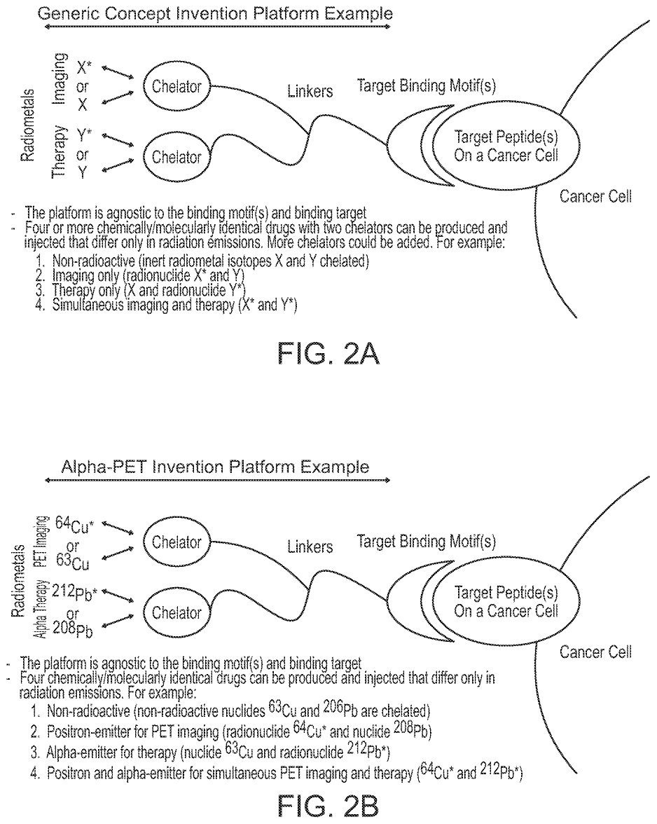

As shown in FIGS. 2A and 2B, depending on the desired use of the conjugate, different combinations of imaging isotopes and radiotherapy isotopes can be selected, resulting in conjugates that differ only in emissions of radiation, but are identical in chemical structure, and therefore identical in binding affinity and biodistribution. For example, for a non-radioactive conjugate, inert radiometal isotopes (e.g., ⁶³Cu and ²⁰⁸Pb) can be selected for chelation with the two or more chelators. For an imaging only conjugate, an imaging isotope (e.g., ⁶⁴Cu) and an inert radiotherapy isotope (e.g., ²⁰⁸Pb) can be selected for chelation with the two or more chelators. For a therapy only conjugate, a radiotherapy isotope (e.g., ²¹²Pb) and an inert imaging isotope (e.g., ⁶³Cu) can be selected for chelation with the two or more chelators. In some embodiments, an imaging only conjugate and a therapy only conjugate can be prepared such that the desired dose (radioactively speaking) of each radioisotope is administered at the time of injection. For a conjugate that can be used for simultaneous imaging and therapy, an imaging isotope (e.g., ⁶⁴Cu) and a radiotherapy isotope (e.g., ²¹²Pb) can be selected for chelation with the two or more chelators.

In some embodiments, a fluorescent dye is used instead of an imaging isotope. Non-limiting examples of fluorescent dyes such as coumarin, cyanine, carboxyfluorescein, quantum dots, green fluorescent protein (GFP), yellow fluorescent protein, red fluorescent protein, phycobiliproteins (e.g., phycoerythrin, phycocyanin, or allophycocyanin), a xanthene derivative such as fluorescein or fluorescein isthiocyanate (FITC), rhodamine, Oregon green, eosin, and Texas red, a cyanine derivative such as cyanine, indocarbocyanine, oxacarbocyanine, thiacarbocyanine, and merocyanine; a squaraine derivative and ring-substituted squaraines, including Seta and Square dyes; squaraine rotaxane derivatives (e.g., Tau dyes), naphthalene derivatives (e.g., dansyl and prodan derivatives); a coumarin derivative, an oxadiazole derivatives (e.g., pyridyloxazole, nitrobenzoxadiazole and benzoxadiazole); an anthracene derivative (e.g., an anthraquinone, including DRAQ5, DRAQ7 and CyTRAK Orange); a pyrene derivative (e.g., cascade blue); an oxazine derivatives (e.g., Nile red, Nile blue, cresyl violet, oxazine 170); an acridine derivative (e.g., proflavin, acridine orange, acridine yellow); an arylmethine derivatives (e.g., auramine, crystal violet, malachite green); a tetrapyrrole derivative (e.g., porphin, phthalocyanine, bilirubin); a dipyrromethene derivative (e.g., BODIPY, aza-BODIPY); an amino group (active ester, carboxylate, isothiocyanate, hydrazine), carboxyl groups (carbodiimide), thiol (maleimide, acetyl bromide), or azide (via click chemistry or non-specifically (glutaraldehyde)).

For any of the conjugates, the binding moiety can be one or more small molecules, nanoparticles, liposomes, exosomes, polypeptides (e.g., an antibody or peptide), nucleic acids (e.g., DNA or RNA), oligonucleotides, viruses, or any other targeted biologic that binds to a target molecule on a cell (e.g., a cancer cell). In some cases, the binding moiety is an extracellular vesicle, a virus, or glucose, or a derivative thereof. In some cases, the binding moiety can target a molecule on the surface of a cell (e.g., a cell surface receptor). For example, a small molecule such as a Glu-ureido based prostate specific membrane antigen (PSMA) inhibitor (also referred to as glutamate carboxypeptidase II inhibitors) can be used as a binding moiety. See, e.g., Kopka, et al., J. Nucl. Med., 58(Supplement 2):17S-26S (2017). PSMA (also is referred to as folate hydrolase 1 (FOLH1), FGCP, FOLH, GCP2, PSM, mGCP, GCPII, NAALAD1, or NAALAdase) is a cell membrane peptidase that belongs in the M28B subfamily of the M28 peptidase family. For example, nanoparticles containing a glutamate carboxypeptidase II inhibitor can be used a binding moiety. In some embodiments, a nanoparticle can be a hydrophilic polyethylene glycol corona with small-molecule PSMA targeting ligands, See, for example, Autio, et al., JAMA Oncology, 4(10):1344-1351 (2018). An exosome such as a dendritic cell derived exosome (see, e.g., Xu, et al., Molecular Cancer, 19, 160 (2020)) can be used a binding moiety.

For example, in some embodiments, the binding moiety can be a polypeptide that binds PSMA, a somatostatin receptor, a fibroblast activating protein (FAP) polypeptide, a melanocortin-1 receptor, a B7-H3 protein, a CA19-9 expressing tumor, a cluster of differentiation 37 (CD37), a cluster of differentiation 3 (CD3), a cluster of differentiation 20 (CD20), a c-x-c-motifchemokine receptor 4 (CXCR4), a gastrin releasing peptide receptor (GRPR), a human epidermal growth factor receptor 2 (HER2), a melanocortin 1 receptor (MCIR), a somatostatin receptor 2 (SSTR2), a vascular endothelial growth factor (VEGF), a programmed death-ligand 1 (PD-L1) polypeptide, a tumor associated calcium signal transducer 2 (TROP2) polypeptide, a protein tyrosine kinase 2 (PTK2) polypeptide, an integrin beta 6 (ITGB6) polypeptide, a neurotensin receptor ligand, CD8, or vitamin B-12. See, e.g., Langbein et al., J. Nucl. Med., 60(Supplement 2):13S-19S (2019). For example, the polypeptide can be a somatostatin analog such as Phe1-Tyr3-octreotate (TATE) or Phe1-Tyr3-octreotide (TOC). See, e.g., Stueven et al., Jnt. J. Mol. Sci., 20(12):3049 (2019). In some embodiments, the conjugate includes two different polypeptides. In some embodiments, the polypeptide can be an antibody or an antibody fragment having the ability to bind an antigen. The term “antibody” as used herein includes monoclonal antibodies, polyclonal antibodies, recombinant antibodies, humanized antibodies, chimeric antibodies, nanobodies, or multispecific antibodies (e.g., bispecific antibodies) formed from at least two antibodies. The term “antibody fragment” comprises any portion of the afore-mentioned antibodies, such as their antigen binding or variable regions (e.g., single VH domains). The term “epitope” refers to an antigenic determinant on an antigen to which the paratope of an antibody binds. Epitopic determinants usually consist of chemically active surface groupings of molecules (e.g., amino acid or sugar residues) and usually have specific three-dimensional structural characteristics as well as specific charge characteristics.

Examples of antibody fragments include Fab fragments, Fab′ fragments, F(ab′)₂ fragments, Fv fragments, diabodies, single chain antibody molecules, single VH domains, and other fragments as long as they exhibit the desired capability of binding to the target molecule. An “Fv fragment” is the minimum antibody fragment that contains a complete antigen-recognition and binding site. This region consists of a dimer of one heavy chain variable domain and one light chain variable domain in tight, non-covalent association. It is in this configuration that the three complementarity determining regions (CDRs) of each variable domain interact to define an antigen-binding site on the surface of the VH-VL dimer. Collectively, the six CDR's confer antigen-binding specificity to the antibody. However, even a single variable domain (or half of an Fv comprising only three CDR's specific for an antigen) has the ability to recognize and bind the antigen, although usually at a lower affinity than the entire binding site. The “Fab fragment” also contains the constant domain of the light chain and the first constant domain (C_{H1}) of the heavy chain. The “Fab fragment” differs from the “Fab′ fragment” by the addition of a few residues at the carboxy terminus of the heavy chain C_{H1} domain, including one or more cysteines from the antibody hinge region. The “F(ab′)₂ fragment” originally is produced as a pair of “Fab′ fragments” which have hinge cysteines between them. Methods of preparing such antibody fragments, such as papain or pepsin digestion, can be performed using any appropriate method.

In some cases, the antibodies can be humanized monoclonal antibodies. Humanized monoclonal antibodies can be produced by transferring mouse complementarity determining regions (CDRs) from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions when treating humans. General techniques for cloning murine immunoglobulin variable domains are described, for example, by Orlandi et al., Proc. Nat'l. Acad. Sci. USA 86:3833 (1989). Techniques for producing humanized monoclonal antibodies are described, for example, by Jones et al., Nature 321:522 (1986); Riechmann et al., Nature 332:323 (1988); Verhoeyen et al., Science 239:1534 (1988); Carter et al., Proc. Nat'l. Acad. Sci. USA 89:4285 (1992); and Sandhu, Crit. Rev. Biotech. 12:437 (1992); Singer et al., J. Immunol. 150:2844 (1993). In some cases, humanization such as super humanization can be used as described by Hwang et al., Methods, 36:35-42 (2005). In some cases, CDR grafting (Kashmiri et al., Methods, 36:25-34 (2005)), human string content optimization (Lazar et al., Mol. Immunol., 44:1986-1998 (2007)), framework shuffling (Dall'Acqua et al., Methods, 36:43-60 (2005); and Damschroder et al., Mol. Immunol., 44:3049-3060 (2007)), and phage display approaches (Rosok et al., J. Biol. Chem., 271:22611-22618 (1996); Radar et al., Proc. Natl Acad Sci. USA, 95:8910-8915 (1998); and Huse et al., Science, 246:1275-1281 (1989)) can be used to obtain antibody preparations that bind to a target molecule. In some cases, fully human antibodies can be generated from recombinant human antibody library screening techniques as described, for example, by Griffiths et al., EMBO J, 13:3245-3260 (1994); and Knappik et al., J Mol. Biol., 296:57-86 (2000).

Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, Fab fragments can be produced by enzymatic cleavage of antibodies with papain. In some cases, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. In some cases, an enzymatic cleavage using pepsin can be used to produce two monovalent Fab′ fragments and an Fc fragment directly. These methods are described, for example, by Goldenberg (U.S. Pat. Nos. 4,036,945 and 4,331,647). See also Nisonhoff et al., Arch. Biochem. Biophys. 89:230 (1960); Porter, Biochem. J. 73:119 (1959); Edelman et al., METHODS IN ENZYMOLOGY, VOL. 1, page 422 (Academic Press 1967); and Coligan et al. at sections 2.8.1 2.8.10 and 2.10.1 2.10.4.

An antibody can be of the IgA-, IgD-, IgE-, IgG- or IgM-type, including IgG- or IgM-types such as, without limitation, IgG1-, IgG2-, IgG3-, IgG4-, IgM1- and IgM2-types. For example, in some cases, an antibody is of the IgG1-, IgG2- or IgG4-type.

In some embodiments, the antibody can be an antibody that binds PSMA. For example, an antibody that binds PSMA can include CDRs that comprise, consist essentially of, or consist of the CDR amino acid sequences set forth in SEQ ID NOs: 1-6. In some cases, an antibody that binds PSMA can have one or more CDRs that are a variant of (e.g., are not 100% identical to) a CDR set forth in any one of SEQ ID NOs:1-6, provided that the antigen binding domain retains the ability to bind to PSMA. For example, one or more CDRs of an antibody that binds PSMA can consist of an amino acid sequence set forth in any one of SEQ ID NOs:1-6, except that the variant polypeptide includes one, two, three, four, or five amino acid substitutions within the articulated sequence of the sequence identifier (e.g., any one of SEQ ID NOs:1-6), has one, two, three, four, or five amino acid residues preceding the articulated sequence of the sequence identifier (e.g., any one of SEQ ID NOs:1-6), and/or has one, two, three, four, or five amino acid residues following the articulated sequence of the sequence identifier (e.g., any one of SEQ ID NOs:1-6), provided that the antibody retains the ability to bind PSMA. Examples of CDR amino acid sequences that comprise, consist essentially of, or consist of the CDR amino acid sequences set forth in SEQ ID NOs: 1-6 and can be used in an antibody that binds PSMA include, without limitation, those amino acid sequences shown in Table 1 (see, also, Example 17).

TABLE 1
Exemplary CDR sequences for anti-PSMA
antibodies. VL refers to variable light
chain, and VH refers to variable heavy chain.

| CDR | Amino Acid Sequence | SEQ ID NO: |
|---|---|---|
| VL CDR1 | QSINNY | 1 |
| | QGIRND | 49 |
| | SASSSISSNYLH | 50 |
| | RASQGISSALA | 51 |
| | RASQDISSALA | 52 |
| | RASQSVSSYLA | 53 |
| | KASQDVGTAVD | 54 |
| | KASENVGTYVS | 55 |
| | RASESIDSYDNTFMH | 56 |
| | KASQNVGSDVA | 57 |
| | KSISKY | 58 |
| VL CDR2 | TAS | 2 |
| | GAS | 59 |
| | RTSNLAS | 60 |
| | DASSLES | 61 |
| | DASNRAT | 62 |
| | WASTRHT | 63 |
| | GASNRFT | 64 |
| | RASILES | 65 |
| | STSYRYS | 66 |
| | SGS | 67 |
| VL CDR3 | QQSFSTPPIT | 3 |
| | LQHNSHPYT | 68 |
| | QQGSYIPFT | 69 |
| | QQNSYPLT | 70 |
| | QQFNSYPLT | 71 |
| | QQRSNWPLFT | 72 |
| | GQSYTFPYT | 73 |
| | HQSIEDPYT | 74 |
| | QQYNSYPLT | 75 |
| | QQHIEYPWT | 76 |
| VH CDR1 | GFTFADFT | 4 |
| | GFTFITYG | 77 |
| | GFTFSNYN | 78 |
| | GFSFSGYG | 79 |
| | GFTFSSYG | 80 |
| | GFTFSDFYMY | 81 |
| | SYAMH | 82 |
| | SNWIG | 83 |
| | SNWIG | 84 |
| | NYWIG | 85 |
| | GYTFTEYTIH | 86 |
| | GFTFSNYWMN | 87 |
| | GYTFGTYVMH | 88 |
| | GFSLTAYGIN | 89 |
| | SGYTFTDYYMH | 90 |
| VH CDR2 | ISWNSNSI | 5 |
| | IYYDESNK | 91 |
| | ISTGSSDI | 92 |
| | MSYDGSNK | 93 |
| | TWYDGSNK | 94 |
| | ISYAGNNK | 95 |
| | TISDGGGYTSYPDSVKG | 96 |
| | VISYDQNNKYYADSVK | 97 |
| | IIYPQDSDTRYSPSFQ | 98 |
| | IIYPGDSDTRYSPSFQ | 99 |
| | IIYPGDSDTRYSPSFQQ | 100 |
| | NINPNNGGTTYNQKED | 101 |
| | EIRSQSNNFATHYAESVKG | 102 |
| | YINPYNDVTRYNGKFKG | 103 |
| | VIWPCGNTDYNSTLKS | 104 |
| | YFNPYNDYTR | 105 |
| VH CDR3 | VKDRSGYSRF | 6 |
| | YYGMDV | 106 |
| | ARAPRVAVEE | 107 |
| | DYSYYYGMDV | 108 |
| | ARDIIGTTRD | 109 |
| | AKDGYYDFLTFDYTLDY | 110 |
| | ARDGNWGGPYWYFDL | 111 |
| | AKDPYYDFLTGSDYFDY | 112 |
| | GLWLRDALDY | 113 |
| | AVPWQSRYYYYQMDV | 114 |
| | QTGFLWSSDL | 115 |
| | QTGFLWSFDL | 116 |
| | PGYTSSWTSFDY | 117 |
| | GWNFDY | 118 |
| | RWNNF | 119 |
| | GENWYYFDS | 120 |
| | DSYGNFKRGWFDF | 121 |
| | CARSDGYYDAMDYW | 122 |

In some embodiments, an antibody that binds PSMA can be as described elsewhere. See, e.g., U.S. Pat. No. 10,179,819, International Patent Application Publication No. WO 2018/129284, International Patent Application Publication No. WO 2002/098897, U.S. Patent Application Publication No. 2014/0273078, EP Patent Application Publication No. 3192810 A1, CN 108699157, EP Patent No. 2,363,404, U.S. Patent Application Publication No. 2014/0234215, International Patent Application Publication No. WO 2005/094882, U.S. Pat. Nos. 7,666,414, 8,114,965, 8,470,330, International Patent Application Publication No. WO 2014/4057113, U.S. Pat. Nos. 9,242,012, 10,179,819, and 9,782,478.

In some embodiments, the antibody that binds PSMA can be the J591 monoclonal antibody or a humanized J591 monoclonal antibody. See, e.g., Milowsky et al., J. Nucl. Med., 50:606-11 (2009). A fully human monoclonal antibody that binds PSMA also can be used. See, e.g., Ma et al., Clin. Cancer Res., 12(8):2591-6 (2006).

In some embodiments, the antibody can be an antibody that binds a somatostatin receptor polypeptide. Examples of somatostatin receptor polypeptides include, without limitation, sstrl receptor polypeptides, sstr2a receptor polypeptides, sstr2b receptor polypeptides, sstr3 receptor polypeptides, sstr4 receptor polypeptides, and sstr5 receptor polypeptides. For example, an antibody that binds a somatostatin receptor can include CDRs that comprise, consist essentially of, or consist of the CDR amino acid sequences set forth in SEQ ID NOs: 7-12. In some cases, an antibody that binds a somatostatin receptor provided herein can have one or more CDRs that are a variant of (e.g., are not 100% identical to) a CDR set forth in any one of SEQ ID NOs: 7-12, provided that the antigen binding domain retains the ability to bind to a somatostatin receptor. For example, one or more CDRs of an antibody that binds a somatostatin receptor provided herein can consist of an amino acid sequence set forth in any one of SEQ ID NOs: 7-12, except that the variant polypeptide includes one, two, three, four, or five amino acid substitutions within the articulated sequence of the sequence identifier (e.g., any one of SEQ ID NOs:7-12), has one, two, three, four, or five amino acid residues preceding the articulated sequence of the sequence identifier (e.g., any one of SEQ ID NOs: 7-12), and/or has one, two, three, four, or five amino acid residues following the articulated sequence of the sequence identifier (e.g., any one of SEQ ID NOs:7-12), provided that the antigen binding domain retains the ability to bind to a somatostatin receptor. Examples of CDR amino acid sequences that comprise, consist essentially of, or consist of the CDR amino acid sequences set forth in SEQ ID NOs: 7-12 and can be used in an antibody that binds a somatostatin receptor include, without limitation, those amino acid sequences shown in Table 2 (see, also, Example 17).

TABLE 2
Exemplary CDR sequences for anti-
somatostatin antibodies.

| CDR | Amino Acid Sequence | SEQ ID NO: |
|---|---|---|
| VL CDR1 | RSSQSLVHSNGNTYLH | 7 |
| | KSSQSLLNSRNRKNYLA | 123 |
| | KSSQSLLNSRTRKNYLA | 124 |
| VL CDR2 | KVSNRFS | 8 |
| | WASTRES | 125 |
| | WASTRAS | 126 |
| VL CDR3 | SQSTHVPFT | 9 |
| | SQSTRVPFT | 127 |
| | KQSYYLWT | 128 |
| VH CDR1 | DYHLN | 10 |
| | DYHMN | 129 |
| | DYGMA | 130 |
| VH CDR2 | IRNKRYGYRTEYSASVKG | 11 |
| | LIRNKANGYRTEYSASVKG | 131 |
| | FISNLGYSIYYADSVKG | 132 |
| | FISNLAYSIYYADSVKG | 133 |
| VH CDR3 | DFYDPFAY | 12 |
| | APYDYDSFYPMDY | 134 |
| | APYDYDSFDPMDY | 135 |

In some embodiments, an antibody that binds a somatostatin receptor can be UMB1, UMB4, UMB5, orUMB7.

In some embodiments, an antibody that binds a somatostatin receptor can be as described elsewhere. See, e.g., International Patent Application Publication No. WO 2018/005706, U.S. Patent Application Publication No. 2009/0016989, U.S. Patent Application Publication No. 2021/0340264, U.S. Pat. No. 11,225,521, NZ749841A, AU2017290086A, CN 201780041351.9A, and Korner et al., Am J Surg Pathol. 2012 February; 36(2):242-52.

In some embodiments, the antibody can be an antibody that binds a FAP polypeptide. For example, an antibody that binds a FAP polypeptide can include CDRs that comprise, consist essentially of, or consist of the CDR amino acid sequences set forth in SEQ ID NOs: 13-18. In some cases, an antibody that binds a FAP polypeptide can have one or more CDRs that are a variant of (e.g., are not 100% identical to) a CDR set forth in any one of SEQ ID NOs: 13-18, provided that the antigen binding domain retains the ability to bind to a FAP polypeptide. For example, one or more CDRs of an antibody that binds a FAP polypeptide can consist of an amino acid sequence set forth in any one of SEQ ID NOs: 13-18, except that the variant polypeptide includes one, two, three, four, or five amino acid substitutions within the articulated sequence of the sequence identifier (e.g., any one of SEQ ID NOs:13-18), has one, two, three, four, or five amino acid residues preceding the articulated sequence of the sequence identifier (e.g., any one of SEQ ID NOs: 13-18), and/or has one, two, three, four, or five amino acid residues following the articulated sequence of the sequence identifier (e.g., any one of SEQ ID NOs: 13-18), provided that the antigen binding domain retains the ability to bind to a FAP polypeptide. Examples of CDR amino acid sequences that comprise, consist essentially of, or consist of the CDR amino acid sequences set forth in SEQ ID NOs: 13-18 and can be used in an antibody that binds a FAP polypeptide include, without limitation, those amino acid sequences shown in Table 3 (see, also, Example 17).

TABLE 3
Exemplary CDR sequences for anti-FAP
antibodies.

| CDR | Amino Acid Sequence | SEQ ID NO: |
|---|---|---|
| VL CDR1 | RASQSISSYLN | 13 |
| | KSSQSLLYSRNQKNYLA | 136 |
| | RASQSVSRNYLA | 137 |
| | RASQSVTSSYLA | 138 |
| VL CDR2 | AASSLOS | 14 |
| | WASTRES | 139 |
| | GASSRAT | 140 |
| | GASTRAT | 141 |
| | VGSRRAT | 142 |
| VL CDR3 | QQSYKPYT | 15 |
| | QQSYSTPRT | 143 |
| | QQFSYPLT | 144 |
| | QQSLGYPPT | 145 |
| | QQGQVIPPT | 146 |
| VH CDR1 | SYAMS | 16 |
| | SYAMS | 147 |
| | SYAMS | 148 |
| | SYAMN | 149 |
| | SYAMS | 150 |
| | RYAM | 151 |
| VH CDR2 | AISGSGGTTYYADSVKG | 17 |
| | AISGSGGGTRYADSVKG | 152 |
| | GISGSGGTYYADSVKG | 153 |
| | TISSSGRTYYADSVKG | 154 |
| | GISGGGGSTYYADSVKG | 155 |
| | SISAGGTYYADSVKG | 156 |
| VH CDR3 | QQSYKPYT | 18 |
| | HSSGFHWYFDY | 157 |
| | ISFYPGGTYFDY | 158 |
| | GLVASAPFD | 159 |
| | IAHSRIGWHFDY | 160 |
| | TFSGYAHYDFDY | 161 |

In some embodiments, an antibody that binds a FAP polypeptide can be sibrotuzumab or BMS168.

In some embodiments, an antibody that binds a FAP polypeptide can be as described elsewhere. See, e.g., JP7017599 B2, JP 2009522329 A, U.S. Patent Application Publication No. 2021/0253736, EP 3269740 A1, U.S. Pat. No. 8,999,342, U.S. Patent Application Publication No. 2017/0369592, IL 281739 DO, U.S. Pat. No. 9,481,730, and ES 2348556 T3.

In some embodiments, the antibody can be an antibody that binds a CD3 polypeptide. For example, an antibody that binds a CD3 polypeptide can include CDRs that comprise, consist essentially of, or consist of the CDR amino acid sequences set forth in SEQ ID NOs: 19-24. In some cases, an antibody that binds a CD3 polypeptide can have one or more CDRs that are a variant of (e.g., are not 100% identical to) a CDR set forth in any one of SEQ ID NOs: 19-24, provided that the antigen binding domain retains the ability to bind to a CD3 polypeptide. For example, one or more CDRs of an antibody that binds a CD3 polypeptide can consist of an amino acid sequence set forth in any one of SEQ ID NOs: 19-24, except that the variant polypeptide includes one, two, three, four, or five amino acid substitutions within the articulated sequence of the sequence identifier (e.g., any one of SEQ ID NOs: 19-24), has one, two, three, four, or five amino acid residues preceding the articulated sequence of the sequence identifier (e.g., any one of SEQ ID NOs: 19-24), and/or has one, two, three, four, or five amino acid residues following the articulated sequence of the sequence identifier (e.g., any one of SEQ ID NOs: 19-24), provided that the antigen binding domain retains the ability to bind to a CD3 polypeptide. Examples of CDR amino acid sequences that comprise, consist essentially of, or consist of the CDR amino acid sequences set forth in SEQ ID NOs: 19-24 and can be used in an antibody that binds a CD3 polypeptide include, without limitation, those amino acid sequences shown in Table 4 (see, also, Example 17).

TABLE 4
Exemplary CDR sequences for anti-CD3
antibodies.

| CDR | Amino Acid Sequence | SEQ ID NO: |
|---|---|---|
| VL CDR1 | RASQSVSSYLA | 19 |
| | WASQGISSYLA | 162 |
| | RASQGISSALA | 163 |
| VL CDR2 | DASNRAT | 20 |
| | GASSRAT | 164 |
| | YASSLOS | 165 |
| | DASSLGS | 166 |
| | DASSLES | 167 |
| VL CDR3 | QQRSNWPPLT | 21 |
| | QQYGSSPIT | 168 |
| | QQYYSTLT | 169 |
| | QQRSNWPWT | 170 |
| | QQFNSYPIT | 171 |
| VH CDR1 | GYGMH | 22 |
| | SYGMH | 172 |
| VH CDR2 | VIWYDGSKKYYVDSVKG | 23 |
| | IIWYDGSKKNYADSVKG | 173 |
| | AIWYNGRKQDYADSVKG | 174 |
| VH CDR3 | QMGYWHFDL | 24 |
| | GTGYNWFDP | 175 |

In some embodiments, an antibody that binds a CD3 polypeptide can be muromonab or blinatumomab.

In some embodiments, an antibody that binds a CD3 polypeptide can be as described elsewhere. See, e.g., CN 1984931 A, EP 1753783 B1, AU 2009/299792 B2, CN 102796199 A, and JP 6817211 B2.

In some embodiments, the antibody can be an antibody that binds a CD20 polypeptide. For example, an antibody that binds a CD20 polypeptide can include CDRs that comprise, consist essentially of, or consist of the CDR amino acid sequences set forth in SEQ ID NOs: 25-30. In some cases, an antibody that binds a CD20 polypeptide can have one or more CDRs that are a variant of (e.g., are not 100% identical to) a CDR set forth in any one of SEQ ID NOs: 25-30, provided that the antigen binding domain retains the ability to bind to a CD20 polypeptide. For example, one or more CDRs of an antibody that binds a CD20 polypeptide can consist of an amino acid sequence set forth in any one of SEQ ID NOs: 25-30, except that the variant polypeptide includes one, two, three, four, or five amino acid substitutions within the articulated sequence of the sequence identifier (e.g., any one of SEQ ID NOs: 25-30), has one, two, three, four, or five amino acid residues preceding the articulated sequence of the sequence identifier (e.g., any one of SEQ ID NOs: 25-30), and/or has one, two, three, four, or five amino acid residues following the articulated sequence of the sequence identifier (e.g., any one of SEQ ID NOs: 25-30), provided that the antigen binding domain retains the ability to bind to a CD20 polypeptide. Examples of CDR amino acid sequences that comprise, consist essentially of, or consist of the CDR amino acid sequences set forth in SEQ ID NOs: 25-30 and can be used in an antibody that binds a CD20 polypeptide include, without limitation, those amino acid sequences shown in Table 5 (see, also, Example 17).

TABLE 5
Exemplary CDR sequences for anti-CD20 antibodies.

| CDR | Amino Acid Sequence | SEQ ID NO: |
|---|---|---|
| VL CDR1 | RASSSVSYIH | 25 |
| | RASSSLSFMH | 176 |
| | RASSSVSYMH | 177 |
| | RASGSVDSFGNSFMH | 178 |
| | RASESVDNFGNSFMH | 179 |
| VL CDR2 | ATSNLAS | 26 |
| | RASNLES | 180 |
| VL CDR3 | QQWTSNPPT | 27 |
| | HQWSSNPLT | 181 |
| | QQSFSNPPT | 182 |
| | QQSYEDPFT | 183 |
| VH CDR1 | SYNMH | 28 |
| | NYGMN | 184 |
| | NVGMN | 185 |
| VH CDR2 | AIYPGNGDTSYNQKFKG | 29 |
| | WINTYTGEPS | 186 |
| | WINTYTGEPA | 187 |
| | AIYPGNGDTSYNQKFK | 188 |
| VH CDR3 | STYYGGDWYFDV | 30 |
| | SHYGSNYVDYFDV | 189 |
| | WYYSNSYWYFDV | 190 |
| | GAYYRYDLGMDY | 191 |
| | STYYGGDWYFNV | 192 |

In some embodiments, an antibody that binds a CD20 polypeptide can be tositumomab, tituximab, ofatumumab, obinutuzumab, ocrelizumab, or ublituximab.

In some embodiments, an antibody that binds a CD20 polypeptide can be as described elsewhere. See, e.g., EP 1740946 B1, U.S. Pat. No. 8,147,832, EP 1692182 B1, EP 2295468 B1, U.S. Patent Application Publication No. 2004/0093621 A1, U.S. Pat. No. 7,744,877, CN 1210307 C, and CN 104558191 A.

In some embodiments, the antibody can be an antibody that binds a CXCR4 polypeptide. For example, an antibody that binds a CXCR4 polypeptide can include CDRs that comprise, consist essentially of, or consist of the CDR amino acid sequences set forth in SEQ ID NOs: 31-36. In some cases, an antibody that binds a CXCR4 polypeptide can have one or more CDRs that are a variant of (e.g., are not 100% identical to) a CDR set forth in any one of SEQ ID NOs: 31-36, provided that the antigen binding domain retains the ability to bind to a CXCR4 polypeptide. For example, one or more CDRs of an antibody that binds a CXCR4 polypeptide can consist of an amino acid sequence set forth in any one of SEQ ID NOs: 31-36, except that the variant polypeptide includes one, two, three, four, or five amino acid substitutions within the articulated sequence of the sequence identifier (e.g., any one of SEQ ID NOs: 31-36), has one, two, three, four, or five amino acid residues preceding the articulated sequence of the sequence identifier (e.g., any one of SEQ ID NOs: 31-36), and/or has one, two, three, four, or five amino acid residues following the articulated sequence of the sequence identifier (e.g., any one of SEQ ID NOs: 31-36), provided that the antigen binding domain retains the ability to bind to a CXCR4 polypeptide. Examples of CDR amino acid sequences that comprise, consist essentially of, or consist of the CDR amino acid sequences set forth in SEQ ID NOs: 31-36 and can be used in an antibody that binds a CXCR4 polypeptide include, without limitation, those amino acid sequences shown in Table 6 (see, also, Example 17).

TABLE 6
Exemplary CDR sequences for anti-CXCR4 antibodies.

| CDR | Amino Acid Sequence | SEQ ID NO: |
|---|---|---|
| VL CDR1 | QSLYNSRTRKNY | 31 |
| | QSFNSRTRKNY | 193 |
| | KSSQSLFNSRTRKNSL | 194 |
| | TGTISDVGGHNFVS | 195 |
| | TGNSNNVGNQGAA | 196 |
| | QGDSLRKFFAS | 197 |
| | RASQSVNTNLA | 198 |
| | QGDSLRSYYAS | 199 |
| | SGSRSNIGSNTVN | 200 |
| | SGSRSNIGGNTVN | 201 |
| VL CDR2 | WAS | 32 |
| | WASARDS | 202 |
| | WASIRES | 203 |
| | EVTKRPA | 204 |
| | RNNNRPS | 205 |
| | GKNSRPS | 206 |
| | GASSRAT | 207 |
| | GKNNRPS | 208 |
| | TNNQRPS | 209 |
| | ANNQRPS | 210 |
| VL CDR3 | KQSYNLRT | 33 |
| | MQSFNLRT | 211 |
| | KQSTNLRT | 212 |
| | SSYGGSNDVI | 213 |
| | SAWDNRLKTYV | 214 |
| | NSRDSRDNHQV | 215 |
| | QHYGSSPLT | 216 |
| | NSRSGSQRV | 217 |
| | LSFDSSLTSYV | 218 |
| | AAWDDNLSGHW | 219 |
| VH CDR1 | TDYY | 34 |
| | DNY | 220 |
| | GFTFTDNY | 221 |
| | GFSLTDYG | 222 |
| | DNTMS | 223 |
| | DTQVY | 224 |
| | SYGMH | 225 |
| | SNFVAWN | 226 |
| | SYGIS | 227 |
| | SYPMH | 228 |
| | NYGLH | 229 |
| | RYGMH | 230 |
| | SYAMS | 231 |
| | IHTMS | 232 |
| | DYAIG | 233 |
| | GYTIA | 234 |
| | PSAMA | 235 |
| | DYAMS | 236 |
| | MG | 237 |
| | LNAMG | 238 |
| | VNDMG | 239 |
| | YYTIG | 240 |
| | YHAIV | 241 |
| | NYAMG | 242 |
| | INAMG | 243 |
| VH CDR2 | IRNKANGYTT | 35 |
| | IWGDGTT | 244 |
| | FIRNKANGYTTDYSASVRG | 245 |
| | MIWGDGTTDYNSALKS | 246 |
| | VISYDGSNKYYADSVKG | 247 |
| | RTYYRSRWYNDYAVSVQS | 248 |
| | WISAYNGNTNYAQKLQG | 249 |
| | VISSDGRNKYYPDSVKG | 250 |
| | NIKQDGSEKYYVDSVKG | 251 |
| | VISHDGTKKYYADSVKG | 252 |
| | LISYDGSKTFYGESVKG | 253 |
| | GIKSSGDSTRYAGSVKG | 254 |
| | TIKPSGDTTNYANAVKG | 255 |
| | AISWNGGSTDYADSVKG | 256 |
| | CISGSDGSTTYADSVKG | 257 |
| | YHRWSDGANLYADSVKG | 258 |
| | STIWSRGDTYFADSVKG | 259 |
| | AISWNGGSADYADSVKG | 260 |
| | TSRLITDNIIYADSVKG | 261 |
| | GITSSTSTYYADSVKG | 262 |
| | VITSGGGTNYVDSVKG | 263 |
| | CISSSDGSTAYLGSVQG | 264 |
| | CITSRDSITYYASFVKG | 265 |
| | AITRSGVRSGVSAIYGDSVKD | 266 |
| | SITSGGSTVYADSVKG | 267 |
| VH CDR3 | DIPGFAY | 36 |
| | ARDVGSNYFDY | 268 |
| | ARGRQFGFDY | 269 |
| | DVGSNYFDY | 270 |
| | GRQFGFDY | 271 |
| | DLVAAAGTAFDI | 272 |
| | GQHSGFDF | 273 |
| | DTPGIAARRYYYYGMDV | 274 |
| | GGYHDFWSGPDY | 275 |
| | DQVSGITIFGGKWRSPDV | 276 |
| | DGGYCSGGRCYSYGMDV | 277 |
| | ATVTTDGYYYMDV | 278 |
| | SRVSRTGLYTYDN | 279 |
| | DYFGTGV | 280 |
| | DQGPFYSGTYYYTRQYGY | 281 |
| | ARMTTSNDKEYLY | 282 |
| | QYGVGGRVVCPGPYEYDV | 283 |
| | AWMTTSNDKEYLY | 284 |
| | RVRPYGQYDY | 285 |
| | DQGPFYSGTYYYTKGYAY | 286 |
| | RQNYSRSVFGAKDYDY | 287 |
| | DCPDYYSDYECPLED | 288 |
| | YYSSGISTLRS | 289 |
| | BSADSRCSIGSIGFTWLYNN | 290 |
| | BTSMTCPTLIVRENY | 291 |
| | SAIGSGALRRFEYDY | 292 |
| | DGVPEWGKVQYPDTY | 293 |

In some embodiments, an antibody that binds a CXCR4 polypeptide can be ibalizumab, MAB172-100, PA3-305, or hz515H7.

In some embodiments, an antibody that binds a CXCR4 polypeptide can be as described elsewhere. See, e.g., EP 2285833 B1, JP 5749330 B2, U.S. Pat. No. 7,138,496, U.S. Patent Application Publication No. 2005/0002939, EP 2246364 A1, CA 2724409 A1, International Patent Application Publication No. WO 2006/089141, Broussas et al., Mol. Cancer Ther., 2016 August; 15(8):1890-9, International Patent Application Publication No. WO 2000/042074, International Patent Application Publication No. WO 2004/059285, EP 1449850 A1, TW 1469792 B, U.S. Pat. Nos. 8,329,178, 7,892,546, International Patent Application Publication No. WO 2009/138519, International Patent Application Publication No. WO 2009/140124, International Patent Application Publication No. WO 2008/142303, International Patent Application Publication No. WO 2008/060367, U.S. Pat. No. 8,748,107, TW 1469792 B, RU 2636032 C2, U.S. Pat. No. 10,428,151, CN 106211774 B, EP 1871807 B1, U.S. Patent Application Publication No. 2019/0276544, EP 06748215 A, U.S. Pat. No. 8,329,178, and CA 2597717 A.

In some embodiments, the antibody can be an antibody that binds a GRPR polypeptide.

In some embodiments, an antibody that binds a GRPR polypeptide can be ABR-002, sc-398549, A30653.

In some embodiments, an antibody that binds GRPR polypeptide can be as described elsewhere. See, e.g., CA 2089212 C, DE 69637411 T2, EP 0981369 B1, CN 109422810 A, CN 106132993 A, and International Patent Application Publication No. WO 2015/143525.

In some embodiments, the antibody can be an antibody that binds a HER2 polypeptide. For example, an antibody that binds a HER2 polypeptide can include CDRs that comprise, consist essentially of, or consist of the CDR amino acid sequences set forth in SEQ ID NOs: 37-42. In some cases, an antibody that binds a HER2 polypeptide can have one or more CDRs that are a variant of (e.g., are not 100% identical to) a CDR set forth in any one of SEQ ID NOs: 37-42, provided that the antigen binding domain retains the ability to bind to a HER2 polypeptide. For example, one or more CDRs of an antibody that binds a HER2 polypeptide can consist of an amino acid sequence set forth in any one of SEQ ID NOs: 37-42, except that the variant polypeptide includes one, two, three, four, or five amino acid substitutions within the articulated sequence of the sequence identifier (e.g., any one of SEQ ID NOs: 37-42), has one, two, three, four, or five amino acid residues preceding the articulated sequence of the sequence identifier (e.g., any one of SEQ ID NOs: 37-42), and/or has one, two, three, four, or five amino acid residues following the articulated sequence of the sequence identifier (e.g., any one of SEQ ID NOs:37-42), provided that the antigen binding domain retains the ability to bind to a HER2 polypeptide. Examples of CDR amino acid sequences that comprise, consist essentially of, or consist of the CDR amino acid sequences set forth in SEQ ID NOs: 37-42 and can be used in an antibody that binds a HER2 polypeptide include, without limitation, those amino acid sequences shown in Table 7 (see, also, Example 17).

TABLE 7
Exemplary CDR sequences for anti-HER2 antibodies.

| CDR | Amino Acid Sequence | SEQ ID NO: |
|---|---|---|
| VL CDR1 | KASQDVSIGVA | 37 |
| | RASQSVSGSRFTYMH | 294 |
| | RASQDVNTAVA | 295 |
| | KASQDVSTAVA | 295 |
| VL CDR2 | SASYRYS | 38 |
| | SASYRES | 296 |
| | SASYLYS | 297 |
| | SASYLES | 298 |
| | SASYRET | 299 |
| | SASYLYT | 300 |
| | SASYLET | 301 |
| | YASILES | 302 |
| | SASYRYT | 303 |
| | SASFLYS | 304 |
| | SASFRYT | 305 |
| VL CDR3 | QQYYIYPYT | 39 |
| | QHSWEIPPWT | 306 |
| VH CDR1 | GFTFTDYTMD | 40 |
| | GFTFTDYTMS | 307 |
| | GYWMN | 308 |
| | GFNIKDTYIH | 309 |
| | GFTFNDYTMD | 310 |
| VH CDR2 | DVNPNSGGSIYNQRFKG | 41 |
| | MIHPLDAEIRANQKFR | 311 |
| | DVNPNSGGSIYNQRFK | 312 |
| | RIYPTNGYTRYADSVK | 313 |
| | DVNPNSGGSIVNRRFK | 314 |
| VH CDR3 | NLGPSFYFDY | 42 |
| | GTYDGGFEY | 315 |
| | WGGDGFYAMDY | 316 |
| | NLGPFFYFDY | 317 |

In some embodiments, an antibody that binds a HER2 polypeptide can be trastuzumab, pertuzumab, margetuximab, ZW25, or zumuzumab.

In some embodiment, the antibody that binds a HER2 polypeptide can be described elsewhere. See, e.g., Jones et al., Nature, 321, 522-525 (1986), CN 105829346 B, CN 107001479 B, KR 2014/0032004 A, AU 2005/32520, TW 1472339 B, CN 102167742 B, ES 2640449 T3, KR 20170055521 A, CN 111741979 A, International Patent Application Publication No. WO 2021/097220, and CN 107001479 B.

In some embodiments, the antibody can be an antibody that binds a MCR1 polypeptide.

In some embodiments, an antibody that binds a MCR1 polypeptide can be ARC0638 or EPR6530.

In some embodiments, the antibody can be an antibody that binds a VEGF polypeptide. Examples of VEGF polypeptides include VEGF1, VEGFB, VEGFC, and VEGFD. For example, an antibody that binds a VEGF polypeptide can include CDRs that comprise, consist essentially of, or consist of the CDR amino acid sequences set forth in SEQ ID NOs: 43-48. In some cases, an antibody that binds a VEGF polypeptide can have one or more CDRs that are a variant of (e.g., are not 100% identical to) a CDR set forth in any one of SEQ ID NOs: 43-48, provided that the antigen binding domain retains the ability to bind to a VEGF polypeptide. For example, one or more CDRs of an antibody that binds a VEGF polypeptide can consist of an amino acid sequence set forth in any one of SEQ ID NOs: 43-48, except that the variant polypeptide includes one, two, three, four, or five amino acid substitutions within the articulated sequence of the sequence identifier (e.g., any one of SEQ ID NOs: 43-48), has one, two, three, four, or five amino acid residues preceding the articulated sequence of the sequence identifier (e.g., any one of SEQ ID NOs: 43-48), and/or has one, two, three, four, or five amino acid residues following the articulated sequence of the sequence identifier (e.g., any one of SEQ ID NOs: 43-48), provided that the antigen binding domain retains the ability to bind to a MCR1 polypeptide. Examples of CDR amino acid sequences that comprise, consist essentially of, or consist of the CDR amino acid sequences set forth in SEQ ID NOs: 43-48 and can be used in an antibody that binds a VEGF polypeptide include, without limitation, those amino acid sequences shown in Table 8 (see, also, Example 17).

TABLE 8
Exemplary CDR sequences for anti-VEGF antibodies.

| CDR | Amino Acid Sequence | SEQ ID NO: |
|---|---|---|
| VL CDR1 | SASQDISNYLN | 43 |
| | RASQDVSTAVA | 318 |
| | RASQVIRRSLA | 319 |
| | RASQSYAYAVA | 320 |
| | CRASQASYYDVA | 321 |
| VL CDR2 | FTSSLHS | 44 |
| | SASFLYS | 322 |
| | AASNLAS | 323 |
| | DASYLYS | 324 |
| | AASYLYS | 325 |
| VL CDR3 | QQYSTVPWT | 45 |
| | QQSYTTPPT | 326 |
| | QQSNTSPLT | 327 |
| | QQAYSSPDT | 328 |
| | CQQYYYAPAT | 329 |
| VH CDR1 | GYTFTNYGMN | 46 |
| | GYDFTNYGMN | 330 |
| | GYTFTHYGMN | 331 |
| | GYDFTHYGMN | 332 |
| | ASWIH | 333 |
| | GSWIF | 334 |
| | GFAISDYDIH | 335 |
| | DYDIH | 336 |
| VH CDR2 | WLNTYTGEPTYAADFKR | 47 |
| | WINTYTGEPTYAADFKR | 337 |
| | AIYPYSGYTNYADSVKG | 338 |
| | AIWPFGGYTHYADSVKG | 339 |
| | DIAPYAGATAYADSVKG | 340 |
| | AIAPYSGSTYYADSVK | 341 |
| VH CDR3 | YPYYYGSSHWYFDV | 48 |
| | WGHSTSPWAMDY | 342 |
| | SSYAYYAAMDY | 343 |
| | SYAYYSAMDY | 344 |

In some embodiments, an antibody that binds a VEGF polypeptide can be bevacizumab, ranibizumab, brolucizumab, or faricimab.

In some embodiments, the antibody can be an antibody that binds a PD-L1 polypeptide. For example, an antibody that binds a PD-L1 polypeptide can include CDRs that comprise, consist essentially of, or consist of the CDR amino acid sequences set forth in SEQ ID Nos: 345-350. In some cases, an antibody that binds a PD-L1 polypeptide can have one or more CDRs that are a variant of (e.g., are not 100% identical to) a CDR set forth in any one of SEQ ID NOs: 345-350, provided that the antigen binding domain retains the ability to bind to a PD-L1 polypeptide. For example, one or more CDRs of an antibody that binds a PD-L1 polypeptide can consist of an amino acid sequence set forth in any one of SEQ ID NOs: 345-350, except that the variant polypeptide includes one, two, three, four, or five amino acid substitutions within the articulated sequence of the sequence identifier (e.g., any one of SEQ ID NOs: 345-350), has one, two, three, four, or five amino acid residues preceding the articulated sequence of the sequence identifier (e.g., any one of SEQ ID NOs: 345-350), and/or has one, two, three, four, or five amino acid residues following the articulated sequence of the sequence identifier (e.g., any one of SEQ ID NOs: 345-350), provided that the antigen binding domain retains the ability to bind to a PD-L1 polypeptide. Examples of CDR amino acid sequences that comprise, consist essentially of, or consist of the CDR amino acid sequences set forth in SEQ ID NOs: 345-350 and can be used in an antibody that binds a PD-L1 polypeptide include, without limitation, those amino acid sequences shown in Table 9 (see, also, Example 17).

TABLE 9
Exemplary CDR sequences for anti-PD-L1 antibodies.

| CDR | Amino Acid Sequence | SEQ ID NO: |
|---|---|---|
| VL CDR1 | DVSTAVA | 345 |
| | RVSSSYLA | 357 |
| | SVSSYLA | 358 |
| | VGGYNYVS | 359 |
| | TRSSGSIDSNYVQ | 360 |
| VL CDR2 | SASFLY | 346 |
| | DASSRA | 361 |
| | DASNRA | 362 |
| | DVSNRP | 363 |
| | EDNQRPS | 364 |
| VL CDR3 | QQYLYHPAT | 347 |
| | QQYGSLPWT | 365 |
| | QQRSNWPT | 366 |
| | SSYTSSSTRV | 367 |
| | QSYDSNNRHVI | 368 |
| VH CDR1 | DSWIH | 348 |
| | RYWMS | 369 |
| | TYAIS | 370 |
| | SYIMM | 371 |
| | GTFSRSAIS | 372 |
| VH CDR2 | WISPYGGSTY | 349 |
| | NIKQDGSEKY | 373 |
| | GIIPIFGKAH | 374 |
| | SIYPSGGITF | 375 |
| | VIIPAFGEANYAQKFQG | 376 |
| VH CDR3 | RHWPGGF | 350 |
| | EGGWFGELAF | 377 |
| | KFHFVSGSPFGM | 378 |
| | IKLGTVTTV | 379 |
| | ARGRQMFGAGIDF | 380 |

In some embodiments, an antibody that binds a PD-Li polypeptide can be atezolizumab, avelumab, durvalumab, BMS 936559, or cosibelimab.

In some embodiments, the antibody can be an antibody that binds a TROP2 polypeptide. For example, an antibody that binds a VEGF polypeptide can include CDRs that comprise, consist essentially of, or consist of the CDR amino acid sequences set forth in SEQ ID Nos: 35i-356. In some cases, an antibody that binds a TROP2 polypeptide can have one or more CDRs that are a variant of (e.g., are not 100% identical to) a CDR set forth in any one of SEQ ID NOs: 351-356, provided that the antigen binding domain retains the ability to bind to a TROP2 polypeptide. For example, one or more CDRs of an antibody that binds a TROP2 polypeptide can consist of an amino acid sequence set forth in any one of SEQ ID NOs: 351-356, except that the variant polypeptide includes one, two, three, four, or five amino acid substitutions within the articulated sequence of the sequence identifier (e.g., any one of SEQ ID NOs: 351-356), has one, two, three, four, or five amino acid residues preceding the articulated sequence of the sequence identifier (e.g., any one of SEQ ID NOs: 351-356), and/or has one, two, three, four, or five amino acid residues following the articulated sequence of the sequence identifier (e.g., any one of SEQ ID NOs: 351-356), provided that the antigen binding domain retains the ability to bind to a TROP2 polypeptide. Examples of CDR amino acid sequences that comprise, consist essentially of, or consist of the CDR amino acid sequences set forth in SEQ ID NOs: 351-356 and can be used in an antibody that binds a TROP2 polypeptide include, without limitation, those amino acid sequences shown in Table 10 (see, also, Example 17).

TABLE 10
Exemplary CDR sequences for anti-TROP2 antibodies.

| CDR | Amino Acid Sequence | SEQ ID NO: |
|---|---|---|
| VL CDR1 | KASQDVSIAVA | 351 |
| | RASQSISSYLN | 381 |
| | KASQDVSIAV | 382 |
| VL CDR2 | SASYRTT | 352 |
| | AASSLQS | 383 |
| | SASYRYT | 384 |
| VL CDR3 | QQHYITPLT | 353 |
| | QQSYSTPLT | 385 |
| | QQHYITPL | 386 |
| VH CDR1 | YNYGM | 354 |
| | TSYAM | 387 |
| | TNYGM | 388 |
| VH CDR2 | WINTYTGEPTYTDDFKG | 355 |
| | WINTNTGNPTYAQGFTG | 389 |
| | WINTYTGEPTYTDDFK | 390 |
| VH CDR3 | GGFGSSYWYFDV | 356 |
| | EDSNGYKIFDY | 391 |
| | GGFGSSYWYFD | 392 |

In some embodiments, an antibody that binds a TROP2 polypeptide can be sacituzumab or datopotamab.

In some embodiments, the binding moiety can be a liposome. A liposome can be an exosome, vesicle, nanovesicle, or extracellular vesicle (EV). In some embodiments, the liposome can be derived from cells, fluid, or tissues from a mammal. In some embodiments the liposome can be derived from a primate. In some embodiments the liposome can be derived from a human. The liposome can be derived from cells, fluid, or tissues including but not limited to mesenchymal stem cells, adipose cells, T-cells, dendritic cells, macrophages, mast cells, gut epithelium cells, B cells, neurons, oligodendrocytes, astrocytes, bone marrow cells, natural killer cells, plasma, serum, platelet cells, urine, lymph cells, cerebralspinal fluid, cultured cells, milk, saliva, sweat, or ascites fluid. Liposomes can be preferentially targeted to tissues based on the expression of surface polypeptides. In some embodiments, integrin a6b4 and/or integrin a6b1 can target liposomes to cells in the lungs. In some embodiments, integrin avb5 can target liposomes to cells in the liver. In some embodiments, tetraspanin Tspan8 and/or integrin a4 can target liposomes to cells in the pancreas. In some embodiments, integrin b4 can target liposomes to cells in the brain. In some embodiments, CD63 can target liposomes to neurons and glial cells. Liposomes can be selected for the natural presence of these polypeptides or they can be engineered to have these polypeptides on the surface.

In some embodiments, the binding moiety can be a nucleic acid. In some embodiments the nucleic acid can be selected from one of thymine, adenine, guanine, cytosine, and uracil.

In some embodiments, the binding moiety can be an oligonucleotide. In some embodiments the oligonucleotide can comprise DNA or RNA. In some embodiments the oligonucleotide can be an aptamer. Targets to which the aptamer can bind include, but are not limited to, prostate specific membrane antigen (PSMA), Erb-B2 receptor tyrosine kinase 2 (HER2), Erb-B2 receptor tyrosine kinase 3 (HER3), programmed cell death protein 1 (PD-1), programmed cell death 1 ligand 1 (PD-L1), vascular endothelial growth factor (VEGF), nucleolin, mucin 1, avβ3 integrin, CD49c, protein tyrosine kinase 7 (PTK7), tumor associated calcium signal transducer 2 (OTROP2), CD20, Tenascin C, oncofetal antigen/immature laminin receptor protein (OFA/iLRP), periostin, platelet derived growth factor (PDGF), or E2F transcription factor 3 (E2F3). The sequence of an aptamer used as a binding moiety may be as described in table below. In some embodiments, the aptamers may be further modified with an inert polymer. In some embodiments, the inert polymer is polyethylene glycol (PEG).

| SEQ ID | | | |
|---|---|---|---|
| NO: | Aptamer Sequence | Target | Indication |
| 601 | 5′- | PSMA | Prostate Cancer |
| | GGGAGGACGATGCGGACCG | | |
| | AAAAAGACCTGACTTCTATA | | |
| | CTAAGTCTACGTTCCCAGAC | | |
| | GACTCCCGA-3′ | | |
| 602 | 5′- | PSMA | Prostate Cancer |
| | GGGAGGACGATGCGGATCA | | |
| | GCCATGTTTACGTCACTCCT | | |
| | TGTCAATCCTCATCGGCAGA | | |
| | CTCGCCCGA-3′ | | |
| 603 | 5′- | PSMA | Prostate Cancer |
| | GGGAGGACGAUGCGGAUCA | | |
| | GCCAUGUUUACGUCACUCC | | |
| | UUGUCAAUCCUCAUCGGC-3′ | | |
| 604 | 5′- | PSMA | Prostate Cancer |
| | GGGAGGACGAUGCGGAUCA | | |
| | GCCAUGUUUACGUCACUCC | | |
| | U-3′ | | |
| 605 | 5′- | PSMA | Prostate Cancer |
| | AGCGTCGAATACCACTACAC | | |
| | ACCACATCCGTCTTACTCCC | | |
| | CAATTACAGACCACGAGCTC | | |
| | CATTAG-3′ | | |
| 606 | 5′- | PSMA | Prostate Cancer |
| | AGCGTCGAATACCACTACTC | | |
| | CACCTTTCCGTCTAACTCCC | | |
| | CACTTTATGACCACGAGCTC | | |
| | CATTAG-3′ | | |
| 607 | 5′- | PSMA | Prostate Cancer |
| | AGCGTCGAATACCACTACCC | | |
| | AACCTTTACCGTCAAACTCC | | |
| | CCACTTTTGACCACGAGCTC | | |
| | CATTAG-3′ | | |
| 608 | 5′- | PSMA | Prostate Cancer |
| | AGCGTCGAATACCACTACTA | | |
| | AGACGCAATTCGACCTTCTT | | |
| | CCCCATTCGACCACGAGCTC | | |
| | CATTAG-3′ | | |
| 609 | 5′- | PSMA | Prostate Cancer |
| | AGCGTCGAATACCACTACAC | | |
| | TTACACCAACTCTATCATCT | | |
| | CCCTTATAGACCACGAGCTC | | |
| | CATTAG-3′ | | |
| 610 | 5′- | PSMA | Prostate Cancer |
| | AGCGTCGAATACCACTACTT | | |
| | ACATCCCTACGAACAAGCGG | | |
| | ACTGCAGAGACCACGAGCTC | | |
| | CATTAG-3′ | | |
| 611 | 5′- | PSMA | Prostate Cancer |
| | AGCGTCGAATACCACTACCC | | |
| | GCCATCCGTCAACGTCTTCC | | |
| | CACTATTGACCACGAGCTCC | | |
| | ATTAG-3′ | | |
| 612 | 5′- | PSMA | Prostate Cancer |
| | CAGCGAAAGUUGCGUAUGG | | |
| | GUCACUCGCAGGCACAUGU | | |
| | CAUCUGGGCG-3′ | | |
| 613 | 5′- | HER2/3 | Breast Cancer |
| | AGCGTCGAATACCACTACACACCA | | |
| | CATCCGTCTTACTCCCCAATTACA | | |
| | GACCACGAGCTCCATTAG-3′ | | |
| 614 | 5′- | HER2/3 | Breast Cancer |
| | AGCGTCGAATACCACTACTCCACCT | | |
| | TTCCGTCTAACTCCCCACTTTAT | | |
| | GACCACGAGCTCCATTAG-3′ | | |
| 615 | 5′- | HER2/3 | Breast Cancer |
| | AGCGTCGAATACCACTACCCAACC | | |
| | TTTACCGTCAAACTCCCCACTTTT | | |
| | GACCACGAGCTCCATTAG-3′ | | |
| 616 | 5′- | HER2/3 | Breast Cancer |
| | AGCGTCGAATACCACTACTAAGAC | | |
| | GCAATTCGACCTTCTTCCCCATTC | | |
| | GACCACGAGCTCCATTAG-3′ | | |
| 617 | 5′- | HER2/3 | Breast Cancer |
| | AGCGTCGAATACCACTACACTTAC | | |
| | ACCAACTCTATCATCTCCCTTATA | | |
| | GACCACGAGCTCCATTAG-3′ | | |
| 618 | 5′- | HER2/3 | Breast Cancer |
| | AGCGTCGAATACCACTACTTACATC | | |
| | CCTACGAACAAGCGGACTGCAGA | | |
| | GACCACGAGCTCCATTAG-3′ | | |
| 619 | 5′- | HER2/3 | Breast Cancer |
| | AGCGTCGAATACCACTACCCGCCA | | |
| | TCCGTCAACGTCTTCCCACTATT | | |
| | GACCACGAGCTCCATTAG-3′ | | |
| 620 | 5′- | HER2/3 | Breast Cancer |
| | CAGCGAAAGUUGCGUAUGGGUCA | | |
| | CUCGCAGGCACAUGUCAUCUGGG | | |
| | CG-3′ | | |
| 621 | 5′- | TLS11a | Liver Cancer |
| | ACAGCATCCCCATGTGAACAATCG | | |
| | CATTGTGATTGTTACGGTTTCCGCC | | |
| | TCATGGACGTGCT-3′ | | |
| 622 | 5′- | D3-21 | Prostate Cancer |
| | GGAGGCAACGGAGCGGAGA | | |
| | CATTGACTGAGTGAACGTGT | | |
| | AGTG-3′ | | |
| 623 | 5′- | PD-1 | melanoma, Hodgkin's |
| | GCTACTGTACATCACGCCTC | | lymphoma, renal cell carcinoma |
| | TCCCC-3′ | | nonsmall cell lung cancer |
| 624 | 5′- | PD-1 | melanoma, Hodgkin's |
| | CTACTGTACATCACGCCTCT | | lymphoma, renal cell carcinoma |
| | CCCC-3′ | | nonsmall cell lung cancer |
| 625 | 5′- | PD-1 | melanoma, Hodgkin's |
| | GTACAGTTCCCGTCCCTGCA | | lymphoma, renal cell carcinoma |
| | CTACA-3′ | | nonsmall cell lung cancer |
| 626 | 5′- | PD-1 | melanoma, Hodgkin's |
| | GTACAGTTCCCGTCCTGCAC | | lymphoma, renal cell carcinoma |
| | TACA-3′ | | nonsmall cell lung cancer |
| 627 | 5′- | PD-L1 | Triple negative breast cancer, |
| | ACGGGCCACATCAACTCATT | | melanoma cancer, small cell lung |
| | GATAGACAATGCGTCCACTG | | cancer, non-small cell lung |
| | CCCGT-3′ | | cancer, urothelial cancer, liver |
| | | | cancer |
| 628 | PEG-5′- | VEGF | colon cancer, lung cancer, |
| | CGGAAUCAGUGAAUGCUUA | | glioblastoma, and renal-cell |
| | UACAUCCG-3′-dT-5′ | | carcinoma |
| 629 | 5′- | VEGF | colon cancer, lung cancer, |
| | TGTGGGGGTGGACGGGCCG | | glioblastoma, and renal-cell |
| | GGTAGA-3′ | | carcinoma |
| 630 | 5′- | VEGF | colon cancer, lung cancer, |
| | TGTGGGGGAGGACGGGCGG | | glioblastoma, and renal-cell |
| | GGATGC-3′ | | carcinoma |
| 631 | 5′- | VEGF | colon cancer, lung cancer, |
| | TGTGGGGGTGGACTGGGTGG | | glioblastoma, and renal-cell |
| | GTACC-3′ | | carcinoma |
| 632 | 5′- | VEGF | colon cancer, lung cancer, |
| | TGTGGGGGTGGACGGGCCG | | glioblastoma, and renal-cell |
| | AGTACG-3′ | | carcinoma |
| 633 | 5′- | VEGF | colon cancer, lung cancer, |
| | TGTGGGGATGGATGGGCCG | | glioblastoma, and renal-cell |
| | GGCTGC-3′ | | carcinoma |
| 634 | 5′- | VEGF | colon cancer, lung cancer, |
| | TGTGGGGGTGGTTGGGCGGG | | glioblastoma, and renal-cell |
| | GCTGC-3′ | | carcinoma |
| 635 | 5′- | Nucleolin | Acute Myeloid Leukemia/Renal |
| | GGTGGTGGTGGTTGTGGTGG | | carcinoma/prostate |
| | TGGTGG-3′ | | cancer/glioma/breast |
| | | | cancer/melanoma/non-small cell |
| | | | lung cancer/lung cancer |
| | | | colorectal cancer/colon |
| | | | cancer/cervical cancer |
| 636 | 5′- | Nucleolin | Acute Myeloid Leukemia/Renal |
| | TTTGGTGGTGGTGGTTGTGG | | carcinoma/prostate |
| | TGGTGGTGG-3′ | | cancer/glioma/breast |
| | | | cancer/melanoma/non-small cell |
| | | | lung cancer/lung cancer |
| | | | colorectal cancer/colon |
| | | | cancer/cervical cancer |
| 637 | 5′- | Mucin 1 | primary and metastatic breast |
| | GCAGTTGATCCTTTGGATAC | | cancers |
| | CCTGG-3′ | | |
| 638 | 5′- | Mucin 1 | primary and metastatic breast |
| | GGCGTACGGTAGGCGGGGT | | cancers |
| | CAACTG-3′ | | |
| 639 | 5′- | Mucin 1 | primary and metastatic breast |
| | GAAGTGAAAATGACAGAAC | | cancers |
| | ACAACA-3′ | | |
| 640 | 5′- | Mucin 1 | primary and metastatic breast |
| | GGCTATAGCACATGGGTAAA | | cancers |
| | ACGAG-3′ | | |
| 641 | 5′- | Mucin 1 | primary and metastatic breast |
| | CAAACAATCAAACAGCAGT | | cancers |
| | GGGGTG-3′ | | |
| 642 | 5′- | Mucin 1 | primary and metastatic breast |
| | TACTGCATGCACACCACTTC | | cancers |
| | AACTA-3′ | | |
| 643 | 5′- | Mucin 1 | primary and metastatic breast |
| | GGGTTATATTACTCGGCCGG | | cancers |
| | TGTAA-3′ | | |
| 644 | 5′- | Mucin 1 | primary and metastatic breast |
| | GGCTATAGCACATGGGTAAA | | cancers |
| | ACGAC-3′ | | |
| 645 | 5′- | Mucin 1 | primary and metastatic breast |
| | GGCGTACGGTAGGCGGGGT | | cancers |
| | CAACTG-3′ | | |
| 646 | 5′- | Mucin 1 | primary and metastatic breast |
| | GCTGGGTAAATAGATGATTC | | cancers |
| | CCGGC-3′ | | |
| 647 | 5′- | Mucin 1 | primary and metastatic breast |
| | AGTGGTGGTTTTATAAGACT | | cancers |
| | GATAC-3′ | | |
| 648 | 5′- | Mucin 1 | primary and metastatic breast |
| | TAGCCTTAATGCCTTTGTAA | | cancers |
| | GGCGA-3′ | | |
| 649 | 5′- | Mucin 1 | primary and metastatic breast |
| | CGGTCTACGGAGCCGCGTTA | | cancers |
| | AACTT-3′ | | |
| 650 | 5′- | avβ3 integrin | Colorectal cancer, Melanoma, |
| | GGGAGACAAGAAUAAACGC | | prostate cancer, thyroid cancer, |
| | UCAAUUCAACGCUGUGAAG | | breast cancer, glioblastoma, lung |
| | GGCUUAUACGAGCGGAUUA | | cancer, liver cancer, stomach |
| | CCCUUCGACAGGAGGCUCA | | cancer |
| | CAAACAGGC-3′ | | |
| 651 | 5′- | CD49c | Poorly differentiated cancers |
| | ACACCAAAATCGTCCGTTTC | | including gastric cancers or triple |
| | GTTTTAGTCCGTCTCTTTAGG | | negative breast cancer |
| | GTGT-3′ | | |
| 652 | 5′- | CD49c | Poorly differentiated cancers |
| | AGCAGCGTGGAGGATATGA | | including gastric cancers or triple |
| | TCTGACCGTTTTCGTTTGTTA | | negative breast cancer |
| | TAGACGCTGCT-3′ | | |
| 653 | 5′- | PTK7 | Leukemia, malignant and non- |
| | ATACCAGCTTATTCAATTAG | | malignant melanoma |
| | TCACACTTAGAGTTCTAACT | | |
| | GCTGCGCCGCCGGGAAAAT | | |
| | ACTGTACGGTTAGATAGTAA | | |
| | GTGCAATCT-3′ | | |
| 654 | 5′- | PTK7 | Leukemia, malignant and non- |
| | ATCTAACTGCTGCGCCGCCG | | malignant melanoma |
| | GGAAAATACTGTACGGTTAG | | |
| | A-3′ | | |
| 655 | 5′-A*T*C*T*AA CTG CTG | PTK7 | Leukemia, malignant and non- |
| | CGC CGC CGG GPP GTA CGG | | malignant melanoma |
| | TTA* G*A*- 3′ | | |
| | P = hexaethylene glycol | | |
| | phosphate | | |
| | N* = locked nucleic acid or | | |
| | bridged nucleic acid or | | |
| | inaccessible nucleic acid | | |
| 656 | 5′- | TROP2 | Colorectal cancer, pancreatic, |
| | TGCTGTTGTCACCTGCCTCG | | hilar cholangiocarcinoma, |
| | TCTCCCTCGT-3′ | | cervical cancer, gastric cancer, |
| | | | oral cancer, non small cell lung |
| | | | cancer, esophageal cancer, liver |
| | | | cancer, ovary cancer, breast |
| | | | cancer stage IV, hormone |
| | | | refractory prostate cancer, non |
| | | | small cell lung cancer, small cell |
| | | | lung cancer, ovarian cancer, |
| | | | pancreatic ductal cancer, head |
| | | | and neck cancer, squamous cell |
| | | | cancer, endometrial cancer, |
| | | | pellicular thyroid cancer, |
| | | | glioblastoma, triple negative |
| | | | breast cancer |
| 657 | 5′- | TROP2 | Colorectal cancer, pancreatic. |
| | TTCCCTCCTCTGTTGTTCCCC | | hilar cholangiocarcinoma, |
| | CCTCCTCTC-3′ | | cervical cancer, gastric cancer, |
| | | | oral cancer, non small cell lung |
| | | | cancer, esophageal cancer, liver |
| | | | cancer, ovary cancer, breast |
| | | | cancer stage IV, hormone |
| | | | refractory prostate cancer, non |
| | | | small cell lung cancer, small cell |
| | | | lung cancer, ovarian cancer, |
| | | | pancreatic ductal cancer, head |
| | | | and neck cancer, squamous cell |
| | | | cancer, endometrial cancer, |
| | | | pellicular thyroid cancer, |
| | | | glioblastoma, triple negative |
| | | | breast cancer |
| 658 | 5′- | TROP2 | Colorectal cancer, pancreatic, |
| | TGGGGTGGTGGTGTGGGTGG | | hilar cholangiocarcinoma, |
| | GGGTTTGTTC-3′ | | cervical cancer, gastric cancer, |
| | | | oral cancer, non small cell lung |
| | | | cancer, esophageal cancer, liver |
| | | | cancer, ovary cancer, breast |
| | | | cancer stage IV, hormone |
| | | | refractory prostate cancer, non |
| | | | small cell lung cancer, small cell |
| | | | lung cancer, ovarian cancer, |
| | | | pancreatic ductal cancer, head |
| | | | and neck cancer. squamous cell |
| | | | cancer, endometrial cancer, |
| | | | pellicular thyroid cancer, |
| | | | glioblastoma, triple negative |
| | | | breast cancer |
| 659 | 5′- | CD20 | Non-Hodgkin's Lymphoma |
| | CTCCTCTGACTGTAACCACG | | |
| | CCGTATGTCCGAAATACGGA | | |
| | GAACAGCACTCATATGCAAG | | |
| | CCATACGCGGAGGTGCACGC | | |
| | GCATAGGTAGTCCAGAAGCC-3′ | | |
| 660 | 5′- | Tenascin C | Lung cancer, liver cancer, |
| | GGCTGTTGTGAGCCTCCTCC | | lymphnode cancer |
| | CAGAGGGAAGACTTTAGGTT | | |
| | CGGTTCACGTCCCGCTTATT | | |
| | CTTACTCCC-3′ | | |
| 661 | CCTGCACTTGGCTTGGATTT | Tenascin C | Lung cancer, liver cancer, |
| | CAGAAGGGAGACCC | | lymphnode cancer |
| 662 | TGCGTGTGTAGTGTGTCTGT | OFA/iLRP | acute myeloid leukemia |
| | TGTTTGTATTGTTGTCTATCC | | |
| | TCTTAGGGATTTGGGCGG | | |
| 663 | 5′- | Periostin | Breast cancer, head and neck |
| | ACGAGYCYGGYCCYYCCYY | | cancer, osteosarcoma, glioma, |
| | YGACGACYAYYGYYYGGYA | | ovarian cancer, squamous cell |
| | YCGAYCAACAACAA-3′ | | carcinoma, nasopharyngeal |
| | | | carcinoma, non-small cell lung |
| | | | cancer, pancreatic cancer, |
| | | | prostate cancer, liver cancer, |
| | | | bladder cancer. |
| 664 | 5′- | Periostin | Breast cancer, head and neck |
| | ACGAGYCACACGYYGAYGA | | cancer, osteosarcoma, glioma, |
| | CYGGAYGGYAGYYAAAGAG | | ovarian cancer, squamous cell |
| | GGYGGGGCAACAA- | | carcinoma, nasopharyngeal |
| | 3′; Y,Benzly-d(U)TP | | carcinoma, non-small cell lung |
| | | | cancer, pancreatic cancer, |
| | | | prostate cancer, liver cancer, |
| | | | bladder cancer. |
| 665 | 5′- | Periostin | Breast cancer, head and neck |
| | ACGAGYYGYCGCAYGYGCG | | cancer, osteosarcoma, glioma, |
| | GYYCAGYCYGGYCCYYCAG | | ovarian cancer, squamous cell |
| | CACCGYACAACAA- | | carcinoma, nasopharyngeal |
| | 3′; Y,Benzly-d(U)TP | | carcinoma, non-small cell lung |
| | | | cancer, pancreatic cancer, |
| | | | prostate cancer, liver cancer, |
| | | | bladder cancer. |
| 666 | 5′- | Periostin | Breast cancer, head and neck |
| | ACGAGYCYGGYCCYYCCYC | | cancer, osteosarcoma, glioma, |
| | CCYAAYYGCYGYYGAGGYA | | ovarian cancer, squamous cell |
| | YCGGCYACAACAA- | | carcinoma, nasopharyngeal |
| | 3′; Y,Benzly-d(U)TP | | carcinoma, non-small cell lung |
| | | | cancer, pancreatic cancer, |
| | | | prostate cancer, liver cancer, |
| | | | bladder cancer. |
| 667 | 5′- | PDGF | Pancreatic cancer, squamous cell |
| | TGGGAGGGCGCGTTCTTCGT | | carcinoma, glioblastoma and |
| | GGTTACTTTTAGTCCCG[3′T] | | osteosarcoma |
| 668 | 5′- | PDGF | Pancreatic cancer, squamous cell |
| | CACAGGCTACGGCACGTAG | | carcinoma, glioblastoma and |
| | AGCATCACCATGATCCTGTG | | osteosarcoma |
| | [3′T] | | |
| 669 | 5′- | PDGF | Pancreatic cancer, squamous cell |
| | TACTCAGGGCACTGCAAGCA | | carcinoma, glioblastoma and |
| | ATTGTGGTCCCAATGGGCTG | | osteosarcoma |
| | AGTA[3′T] | | |
| 670 | CACTGAACGAATGGCAACC | E2F3 | Liver cancer, lung cancer, |
| | GCCAAACCCTACCCACACTG | transcription | neuroblastoma |
| | G | factor | |
| | | CXCL12 | multiple myeloma, colorectal |
| | | | cancer, pancreatic cancer |

In some embodiments, the binding moiety can be a virus. The virus can be an adeno-associated virus (AAV), an adenovirus, a maraba virus, a measles virus, a herpes simplex 1 virus, a lentivirus, vesicular stomatitis virus (VSV), newcastle disease virus, myxoma virus, foamy virus, parvovirus, reovirus, or poliovirus. In some embodiments, the AAV can be AAV1, AAV2, AAV3, AAV4, AAV5, AAV6, AAV7, AAV8, AAV9, AAV10, AAV11, or AAV12. In some embodiments, the AAV can have a hybrid capsid. In some embodiments, the AAV with a hybrid capsid can be AAV2/10, AAV2/11, AAVDJ, AAFDJ/8, or AAV2i8. In some embodiments, the virus can be modified to express an additional peptide on its surface. In some embodiments, the binding moiety can be engineered AAVs including but not limited to AAV9-PhP.B or AAV5-PhP.B or AAV-PHP.eB or AAV-PHP. Scan or AAV Serotype Retrograde or AAV Serotype BR1 to be used for targeting brain malignancies. Additionally, above viral particles can be Pegylated as well. The peptides can include, but are not limited to, those described in table below.

| SEQ | | |
|---|---|---|
| ID | | |
| NO: | Peptide | Target |
| 671 | KTLLPTPYC | Plectin - 1 |
| 672 | GE11 | Epidermal growth |
| | | factor (EGFR) |
| 673 | RGD | Integrin avβ3 |
| 674 | RGD + CGGGPKKKRKVGG | Integrin avβ3 |
| 675 | CRQAGFSL | Urokinase-type |
| | | Plasminogen |
| | | activator |
| 676 | CPNFDWDPNNSNAGF | Squamous cell |
| | | carcinoma |
| 677 | TSFAEYWALLSP | Murine double |
| | | minute 2 (MDM2) |
| | | and murine double |
| | | minute X (MDMX) |
| 678 | Bombesin | Gastrin-releasing |
| | (YQRLGNQWAVGHLM) | peptide receptor |
| | | (GRPR) |
| 679 | K237 peptide | VEGFR2 binding |
| | (HTMYYHHYQHHL) | peptide |
| | NT21MP peptide | CXCR4 |
| | 680 (LGASWHRPDKCCLGYQKRPLP) | |
| | Nef-M1 peptide | |
| | 681 (NAACAWLEAQ) | |
| | Peptide R | |
| | 682 (RACRFFC) | |
| | Pentixafor | |
| | 683 (DY-[NMe]DOrn-R-2Nal-G) | |
| | Peptide E5 | |
| | 684 (GGRSFFLLRRIQGCRFRNTVDD) | |
| 685 | AGR peptide | Prostate cancer |
| | (CAGRRSAYC) | lymphatics |
| 686 | REA peptide | Pre-malignant tumor |
| | (CREAGRKAC) | lymphatics |
| 687 | LSD peptide | Tumor lymphatics |
| | (CLSDGKRKC) | |
| 688 | PSMA | PSA |
| 689 | FHK peptide | Tenascin-c |
| | (FHKHKSPALSPV) | |
| 690 | TD peptide | |
| | (ACSSSPSKHCG) | |
| | Brain homing peptide | |
| | 691 (CAGALCY) | |
| | RVG peptide | |
| | 692 | |
| | (YTIWMPENPRPGTPCDIFTNSRGKRASNG) | |
| | Peptide #1 | HER2 |
| | 693 (KCCYSL) | |
| | Peptide #2 | |
| | 694 (FCGDFYACYMDV) | |
| | H6F peptide | |
| | 695 (YLFFVFER) | |
| | Peptide #3 | |
| | 696 (CDTFPYLGWWNPNEYRY) | |
| | Peptide # 4 | |
| | 697 (CKTIYYLGYYNPNEYRY) | |
| | Peptide # 5 | |
| | 698 (LTVSPWY) | |
| | Peptide #6 | |
| | 699 (KLRLEWNR) | |
| | A9 peptide | |
| | 700 (QDVNTAVAW) | |
| | Peptide # 7 | |
| | 701 (CGGGLTVSPWY) | |
| | Peptide # 8 | |
| | 702 (MEGPSKCCYSLALASH) | |
| | Peptide # 9 | |
| | 703 (GTKSKCCYSLRRSS) | |
| | Peptide # 10 | |
| | 704 (CSSSLTVSPWY) | |
| | Peptide # 11 | |
| | 705 (CKTIYYLGYYNPNEYRY) | |
| | Peptide # 12 | |
| | 706 (GGCAKIFGSLAFLKCCYSL) | |
| | Peptide # 13 | |
| | 707 (YHWYGYTPQNVI) | |

### Exemplary Method of Preparing a Conjugate

In some embodiments, a conjugate can be prepared according to methods and procedures similar to those described in PCT/US2022/034086, which is incorporated herein by reference. For example, in some embodiments, one chelator can be DiAmSar and one chelator can be TCMC. In some embodiments, one chelator can be NOTA and one chelator can be TCMC. In some embodiments, the binding moiety is a PSMA peptide. In some embodiments, the PSMA peptide is piflufostat. In some embodiments, the binding moiety is a fibroblast activating protein inhibitor (FAPI). In some embodiments, the FAPI is N-[2-[(2S)-2-cyano-4,4-difluoropyrrolidin-1-yl]-2-oxoethyl]-6-hydroxyquinoline-4-carboxamide. In some embodiments, the binding moiety is a ligand for a somatostatin receptor. In some embodiments, the ligand for the somatostatin receptor is octreotide, pasireotide, vapreotide, lanreotide, somatostatin, edotreotide, or oxodotreotide. In some embodiments, the binding moiety is a CD3 inhibitor. In some embodiments, the binding moiety is a CD20 inhibitor. In some embodiments, the binding moiety is a CXCR4 inhibitor. In some embodiments, the CXCR4 inhibitor is framycetin, plerixafor, baclofen, mavorixafor, or MSX-122. In some embodiments, the binding moiety is a GRPR inhibitor. In some embodiments, the GRPR inhibitor is bombesin, RC-3095, PD 168368, GRPR antagonist 1, GRPR antagonist 2, or PD 176252. Some examples of GRPR antagonists that can be used as described herein are as set forth in Yu et al., Med Chem Res 30, 2069-2089 (2021), which is hereby incorporated by reference. In some embodiments, the binding moiety is a HER2 inhibitor. In some embodiments, the HER2 inhibitor is lapatinib, tesevatinib, varlitinib, tucatinib, afatinib, brigatinib, fostamatinib, zanubrutinib, tucatinib, or neratinib. In some embodiments, the binding moiety is a MC1R ligand. In some embodiments, the MC1R ligand is 4-phenylbutyryl-His-DPhe-Arg-Trp-Gly-Lys(hex-5-ynoyl)-NH2, H-Lys(hex-5-ynoyl)-Tyr-Val-Nle-Gly-His-DNal(2′)-Arg-DTrp-Asp-Arg-Phe-Gly-NH2, H-Lys(hex-5-ynoyl)Tyr-Val-Nle-Gly-His-DNal(2′)-Arg-DPhe-Asp-Arg-Phe-Gly-NH2, adrenocorticotropic hormone, alpha melanocyte-stimulating hormone, beta melanocyte-stimulating hormone, gamma melanocyte-stimulating hormone, or MC1RL. Some further examples of MC1R ligands that can be used as described herein are as set forth in one or more of the following: Tafreshi et al., J. Nucl. Med. 60(8), 1124-1133 (2019); and U.S. Pat. Nos. 8,492,517, 8,933,194, and 11,286,280, which are hereby incorporated by reference. In some embodiments, the binding moiety is a VEGF inhibitor. In some embodiments, the VEGF inhibitor is sunitinib, vatalanib, linifanib, denibulin, pazopanib, axitinib, regorafenib, sorafenib, lenvatinib, nintedanib, polaprezinc, fostamatinib, selpercatinib, or tivozanib. In some embodiments, the binding moiety is a PD-L1 inhibitor. In some embodiments, the PD-L1 inhibitor is AUNP-12, CA-170, (3S,3aR,6S,6aR)-N6-[4-(3-fluorophenyl)-pyrimidin-2-yl]-N3-(2-pyridylmethyl)-2,3,3a,5,6,6a-hexahydrofu, or 1-isopropyl-3-[(3S,5S)-1-methyl-5-[3-(2-naphthyl)-1,2,4-oxadiazol-5-yl]pyrrolidin-3-yl]urea. In some embodiments, the binding moiety is a PTK2 inhibitor. In some embodiments, the PTK2 inhibitor is endostatin, fostamatinib, 7-Pyridin-2-Yl-N-(3,4,5-Trimethoxyphenyl)-7h-Pyrrolo[2,3-D]pyrimidin-2-Amine, 2-({5-Chloro-2-[(2-Methoxy-4-Morpholin-4-Ylphenyl)amino]pyrimidin-4-Yl}amino)-N-Methylbenzamide, GSK2256098, defactinib, or VS-4718. In some embodiments, the binding moiety is an ITGB6 binder. In some embodiments, the ITGB6 binder is the cyclic peptide cyclo(FRGDLAFp(NMe)K) or trivehexin, as described in Quigley et al., Eur. J. Nucl. Med. Mol. Imaging. 49(4), 1136-1147 (2022). Sometimes the binding moiety is 3-fluoro-2,2-dimethylpropionic acid or 2,2-dimethylpropionic acid.

As described herein, a conjugate provided herein can include one or more binding moieties (e.g., one, two, three, four, five, or more binding moieties). In some cases, a binding moiety of a conjugate described herein can have the ability to bind to one or more target molecules. For example, a binding moiety of a conjugate described herein can have the ability to bind to one, two, three, four, five, or more target molecules such as one, two, three, four, five, or more target molecules present on a cell (e.g., a cancer cell).

In some embodiments, a conjugate provided herein having two or more binding moieties can advantageously, for example, bind to antigens present on two different cells (e.g., two different cancer cells), or bind to two different antigens on the same cell (e.g., the same cancer cell). In some embodiments, having more than one binding moiety provides one or more advantages, such as, the conjugate having enhanced uptake and/or increased in vivo stability.

In some embodiments, one or more conjugates described herein can be used to treat a cancer (e.g., prostate cancer, a neuroendocrine cancer, colon cancer, lung cancer, pancreatic cancer, melanoma, or a lymphoid cancer) in a mammal (e.g., a human patient). For example, for treating prostate cancer, a conjugate that includes a binding moiety that targets PSMA or its activity can be used. For treating a neuroendocrine cancer, a conjugate that includes a binding moiety that targets a somatostatin receptor (e.g., a somatostatin analog) can be used. For treating lung cancer, a conjugate that includes a binding moiety that targets the B7-H3 protein can be used. For treating pancreatic cancer, a conjugate that includes a binding moiety that targets C9-19 can be used. For treating melanoma, a conjugate that includes a binding moiety that targets the melanocortin 1 receptor can be used. In some embodiments, the cancer is a metastasized tumor (e.g., in a tissue selected from bone, liver, lung, brain, adrenal gland, and such). In some embodiments, the metastatic tumor is about 1 mm, about 2 mm, about 3 mm, about 5 mm, about 10 mm or 20 mm in size.

In some embodiments, one or more conjugates described herein can be used to treat a non-cancer condition (e.g., a benign tumor, an inflammatory condition, a hematologic process, a histiocytic process, a cystic disease or infection) in a mammal (e.g., a human patient).

In some embodiments, one or more conjugates described herein can be administered to a mammal (e.g., a human patient) once or multiple times over a period of time ranging from days to months to treat a cancer or non-cancer condition in a mammal (e.g., a human patient). In some embodiments, one or more conjugates described herein (e.g., a conjugate that includes two or more chelators covalently attached to a binding moiety via a linker, wherein one of the chelators is a chelator of an isotope used for imaging and one of the chelators is a chelator of an isotope used for radiotherapy, wherein the isotope used for imaging and the isotope used for radiotherapy are each complexed (chelated) to the chelator, and wherein the binding moiety binds to a tumor in the patient) can be formulated into a pharmaceutically acceptable composition for administration to a patient (e.g., a patient identified as having cancer) to treat a cancer within that patient. In some embodiments, a mixture of two conjugates can be administered to, for example, provide a suitable dose (radioactively speaking) of each radioisotope at the time of injection. In such embodiments, the appropriate isotopes can be complexed with the chelators of the two conjugates and mixed at the time of injection to account for the decay at different rates.

In some embodiments, the biodistribution of the conjugate (i.e., location of the conjugate within the mammal) can be determined in the patient (e.g., by PET) by administering a conjugate that includes two or more chelators covalently attached to a binding moiety via a linker, wherein one of the chelators is a chelator of an imaging isotope and one of the chelators is a chelator of a radiotherapy isotope, wherein the imaging isotope is chelated to the chelator, and wherein the binding moiety binds to a tumor in the patient. After the biodistribution is determined, the same conjugate, except having both the imaging isotope and radiotherapy isotope chelated to the chelator, can be administered to the patient. Determining the biodistribution allows the dose of the therapy to be tailored to the patient, reducing side effects. Imaging can be performed after each administration of conjugate to monitor therapy.

A therapeutically effective amount of a conjugate described herein can be formulated together with one or more pharmaceutically acceptable carriers (additives or excipients) and/or diluents. In some embodiments, the additives stabilize against radiolysis. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more conjugates can be designed for oral or parenteral (including subcutaneous, intramuscular, intravenous, intradermal, inhaled/aerosolized, intraarterial, intrathecal, intratumoral, intracystic, peritumoral, intraperitomeal, intraluminal, intrapleural) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, or solutes that render the formulation isotonic with the blood of the intended recipient. The formulations can be presented in unit-dose or multi-dose containers, for example, sealed ampules and vials, and may be stored (e.g., in a freeze dried (lyophilized) condition) requiring only the addition of the sterile liquid carrier, for example, water or saline for injections immediately prior to use. In some embodiments, the formulations can be presented in a form that only requires the addition of a sterile carrier (e.g., water or saline) and the desired radionuclide(s). Extemporaneous injection solutions and suspensions may be prepared from sterile powders, granules, and tablets.

In some cases, a pharmaceutically acceptable composition including one or more conjugates described herein can be administered locally or systemically. For example, a composition provided herein can be administered systemically by intravenous injection or blood infusion. For example, a composition provided herein can be administered locally, e.g., intratumoral, intramuscular, intradermal or subcutaneous). For example, an intraarterial injection can be used to locally direct the composition, e.g., injection into the hepatic artery to target cancer in the liver). In some cases, a composition provided herein can be administered systemically, orally, or by injection to a mammal (e.g., a human patient).

An effective amount of a composition containing one or more conjugates can be any amount that provides an anti-tumor response (e.g., slowing, stopping, or reversing tumor growth by stopping tumor cell multiplication and/or killing tumor cells) without producing significant toxicity to the patient. For example, an effective amount of a conjugate that includes a positron-emitting PET isotope can range from 1 mCi to 20 mCi (e.g., about 1 mCi to about 15 mCi, about 1 mCi to about 10 mCi, about 2 mCi to about 18 mCi, about 3 mCi to about 17 mCi, about 4 mCi to about 18 mCi, about 4 mCi to about 15 mCi, about 5 mCi to about 20 mCi, about 5 mCi to about 15 mCi, about 10 mCi to about 20 mCi, about 15 mCi to about 20 mCi). In some embodiments, an effective amount of a conjugate that includes a beta-emitting isotope can range, for example, from about 10 mCi to 1.5 Ci (1,500 mCi) per cycle (e.g., about 15 mCi to about 1,400 mCi, about 25 mCi to about 1,500 mCi, about 50 mCi to about 1,250 mCi, about 75 mCi to about 1,500 mCi, about 100 mCi to about 1,000 mCi, about 100 mCi to about 1,400 mCi, about 150 mCi to about 1,250 mCi, about 200 mCi to about 1,200 mCi, about 300 mCi to about 1,100 mCi, about 400 mCi to about 1,000 mCi, about 500 mCi to about 1,500 mCi, about 600 mCi to about 1,400 mCi, about 700 mCi to about 1,300 mCi, about 800 mCi to about 1,200 mCi, or about 1,000 mCi to about 1,500 mCi per cycle). In some embodiments, an effective amount of a conjugate that includes a gamma-emitting isotope (e.g., a SPECT agent) can range, for example, from about 0.1 mCi to about 40 mCi (e.g., about 0.2 mCi to about 40 mCi, about 0.5 mCi to about 35 mCi, about 0.5 mCi to about 25 mCi, about 1 mCi to about 35 mCi, about 1 mCi to about 30 mCi, about 2 mCi to about 38 mCi, about 3 mCi to about 30 mCi, about 4 mCi to about 35 mCi, about 4 mCi to about 35 mCi, about 5 mCi to about 40 mCi, about 5 mCi to about 35 mCi, about 5 mCi to about 30 mCi, about 5 mCi to about 25 mCi, about 5 mCi to about 20 mCi, about 10 mCi to about 30 mCi, about 15 mCi to about 40 mCi, about 20 mCi to about 40 mCi, or about 25 mCi to about 40 mCi). In some embodiments, an effective amount of a conjugate that includes an alpha-emitting isotope can range, for example, from about 0.05 mCi to 100 mCi per cycle (e.g., about 0.05 to about 90 mCi, about 0.1 mCi to about 100 mCi, about 0.2 mCi to about 90 mCi, about 0.5 mCi to about 95 mCi, about 0.5 mCi to about 85 mCi, about 1 mCi to about 95 mCi, about 1 mCi to about 85 mCi, about 2 mCi to about 95 mCi, about 3 mCi to about 90 mCi, about 4 mCi to about 85 mCi, about 4 mCi to about 80 mCi, about 5 mCi to about 100 mCi, about 5 mCi to about 85 mCi, about 5 mCi to about 70 mCi, about 5 mCi to about 60 mCi, about 5 mCi to about 50 mCi, about 10 mCi to about 100 mCi, about 15 mCi to about 60 mCi, about 20 mCi to about 80 mCi, or about 25 mCi to about 100 mCi per cycle).

In some embodiments, in which two or more conjugates are to be administered, the effective amount of each conjugate may be different. For example, when it is desired to use an alpha-emitting isotope for therapy and a positon-emitting isotope for imaging, different amounts of the conjugates can be administered.

For example, an effective amount of one or more conjugates described herein can be administered to an average sized human (e.g., about 75-85 kg human) per administration (e.g., per daily, weekly, monthly, bimonthly, or quarterly administration). In some cases, a conjugate can be administered once followed by a rest period of between two and sixteen weeks (e.g., two, three, four, five, six, seven, eight, nine, 10, 11, 12, 13, 14, 15, or 16 weeks) to monitor the patient for adverse effects (e.g., by monitoring complete blood counts, white blood cell count, platelet count, hemoglobin levels, or bone marrow injury) before repeating the administration. Each administration and rest period is referred to as a cycle of therapy.

If a particular mammal fails to respond to a particular amount of therapy drug conjugate, or the calculated amount of drug arriving at a target tumor is too low, then the amount of a conjugate injected in the next cycle can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition may require an increase or decrease in the actual effective amount administered.

The frequency of administration of a conjugate described herein can be any frequency that provides an anti-tumor response (e.g., stopping tumor growth or killing tumor cells) without producing significant toxicity to the mammal. For example, the frequency of administration of a conjugate can be from about once a day, once a month, once every six weeks, once every two months, or about once every three months, or about once every 16 weeks. The frequency of administration of a conjugate described herein can remain constant or can be variable during the duration of treatment (e.g., more frequent administration with less toxicity). As described above, a course of treatment with a composition containing a conjugate can include rest periods. For example, a composition containing one or more conjugates can be administered once followed by a rest period of between two and sixteen weeks (e.g., two, three, four, five, six, seven, eight, nine, 10, 11, 12, 13, 14, 15, or 16 weeks), and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing one or more conjugates can be any duration that provides an anti-tumor response (e.g., stopping tumor growth or killing tumor cells) within a mammal identified as having cancer without producing significant toxicity to the mammal. In some cases, the effective duration can vary from several days to several months. In general, the effective duration for providing an anti-tumor response (e.g., stopping tumor growth or killing tumor cells) within a mammal identified as having cancer can range in duration from about six weeks to about ten months. Multiple factors can influence the actual effective duration used for a particular treatment. For example, an effective duration can vary with the frequency of administration, effective amount, use of multiple treatment agents, route of administration, and severity of the condition being treated.

The invention will be further described in the following examples, which do not limit the scope of the invention described in the claims.

### Exemplary Conjugates for Targeting Cancers

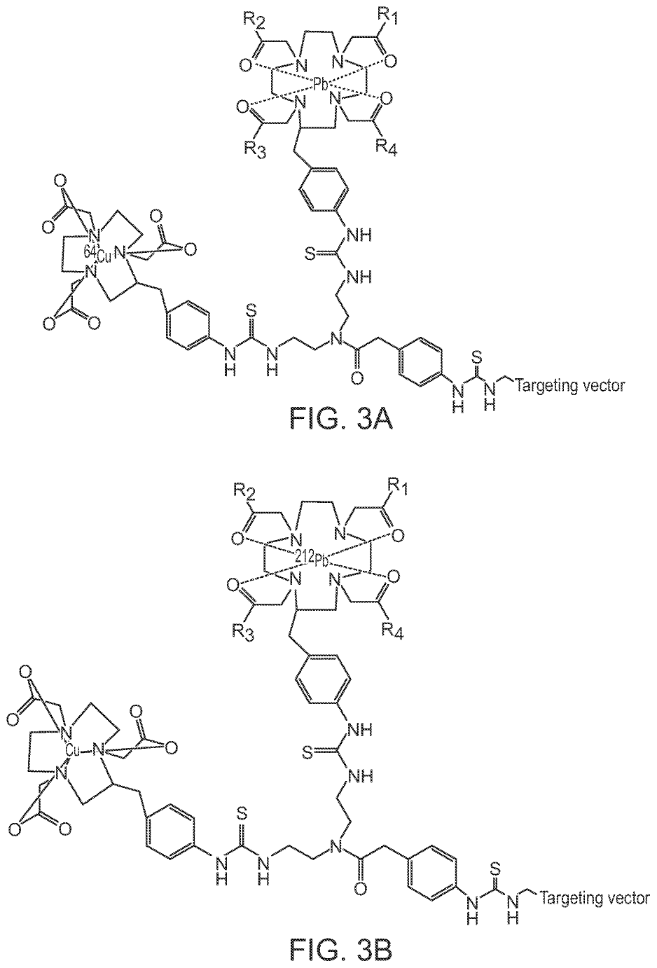

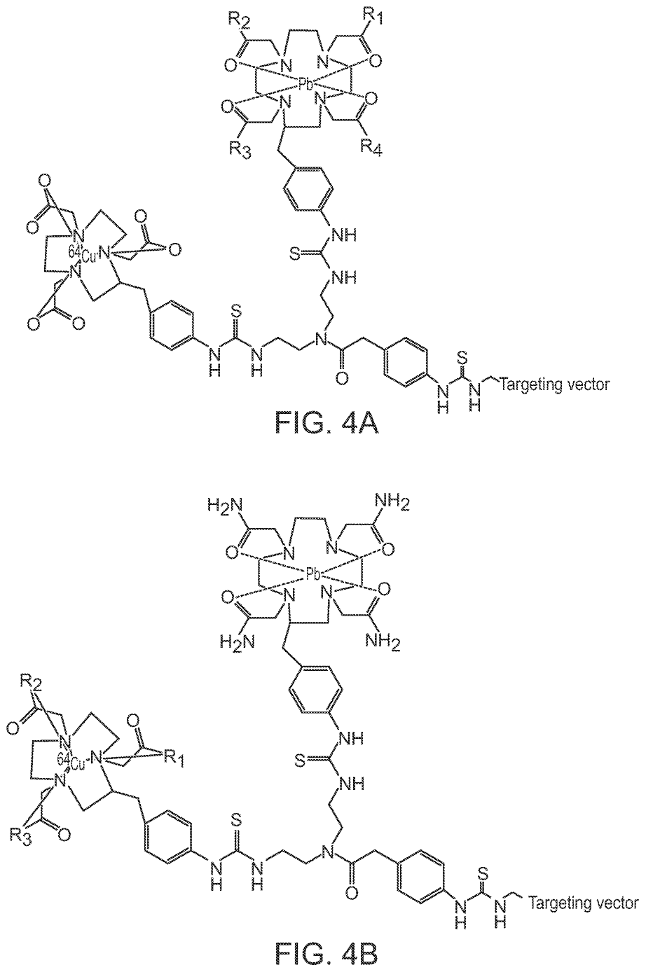

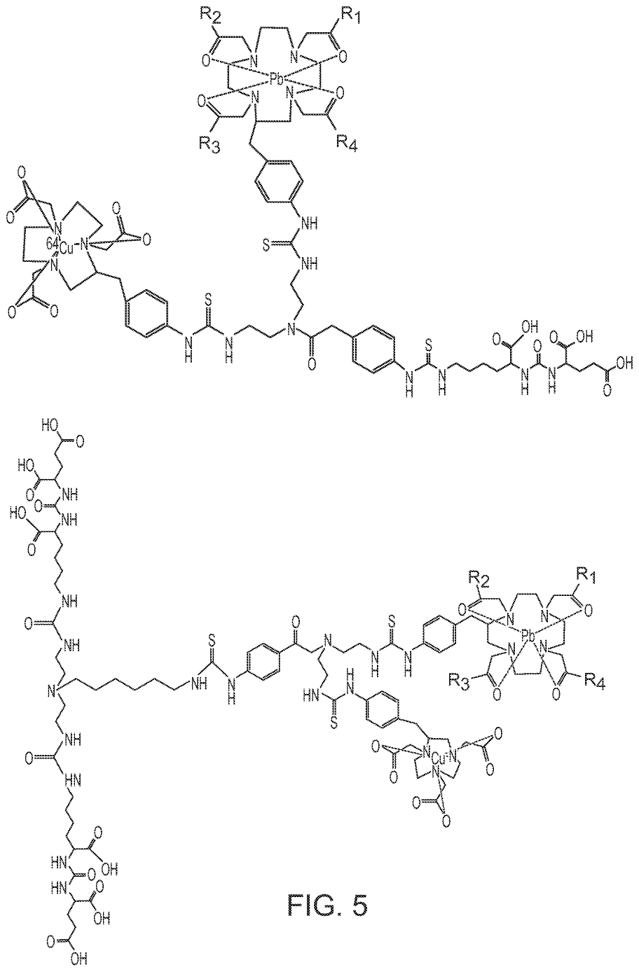

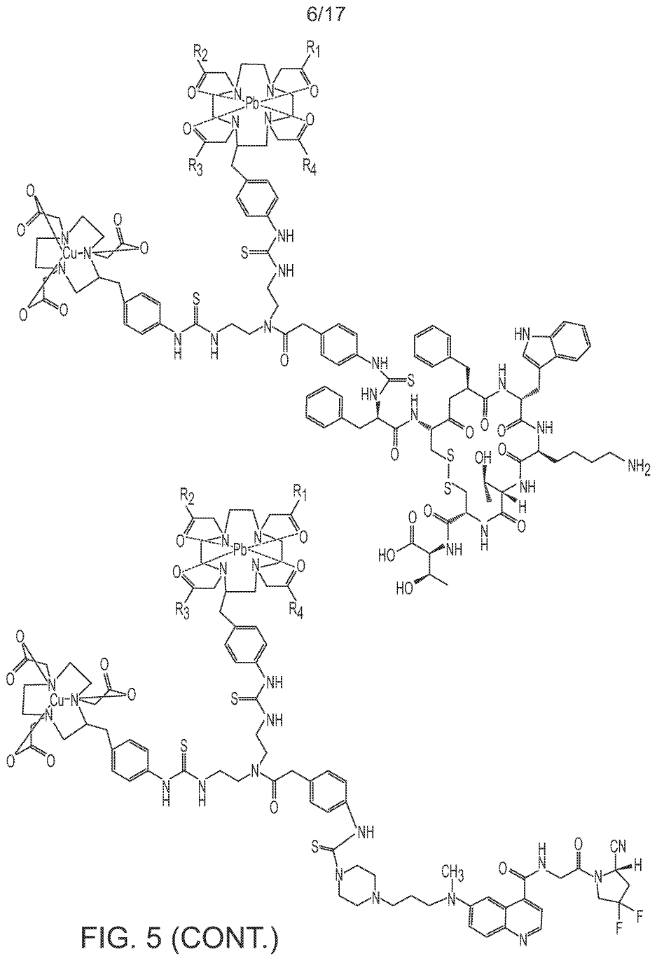

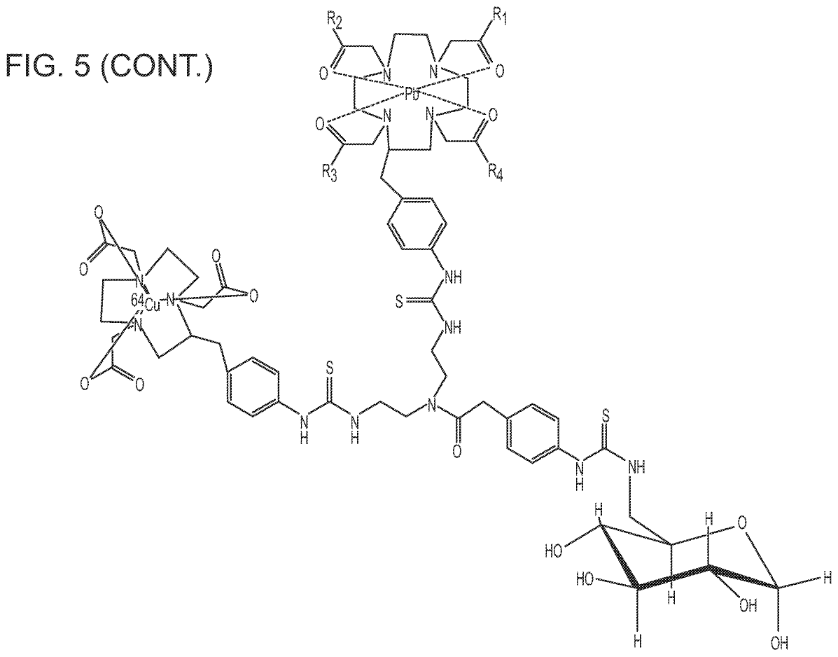

Suitable examples of radiometal-containing conjugates are provided in FIGS. 3A-6. In some cases, these structures can have various combinations with Cu-64, Cu-61, Cu-67, nonradioactive Cu, and Pb-212/Pb-203/Pb-nonradioactive. In some cases, these structures can have various combinations with Zr-89 radioactive and nonradioactive along with Ac-225/Ac-226/Ra-223 both radioactive and nonradioactive isotopes.

The below exemplary structures provide conjugates prior to complexation with metal or metals.

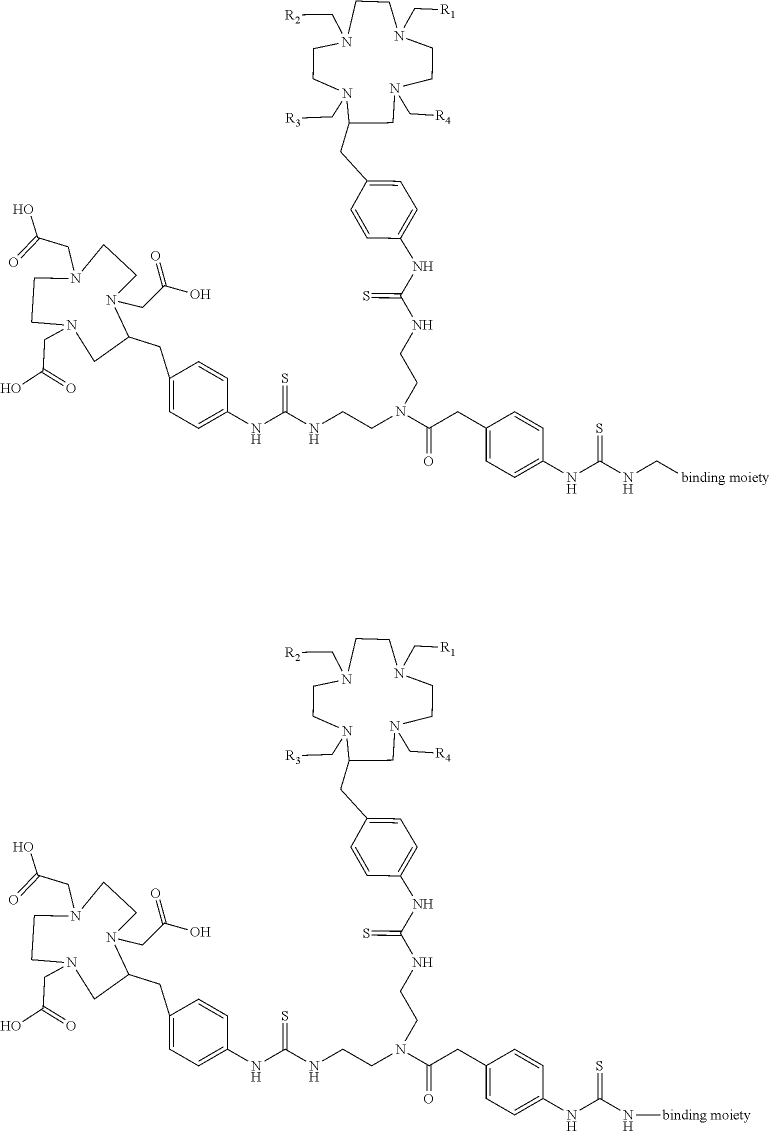

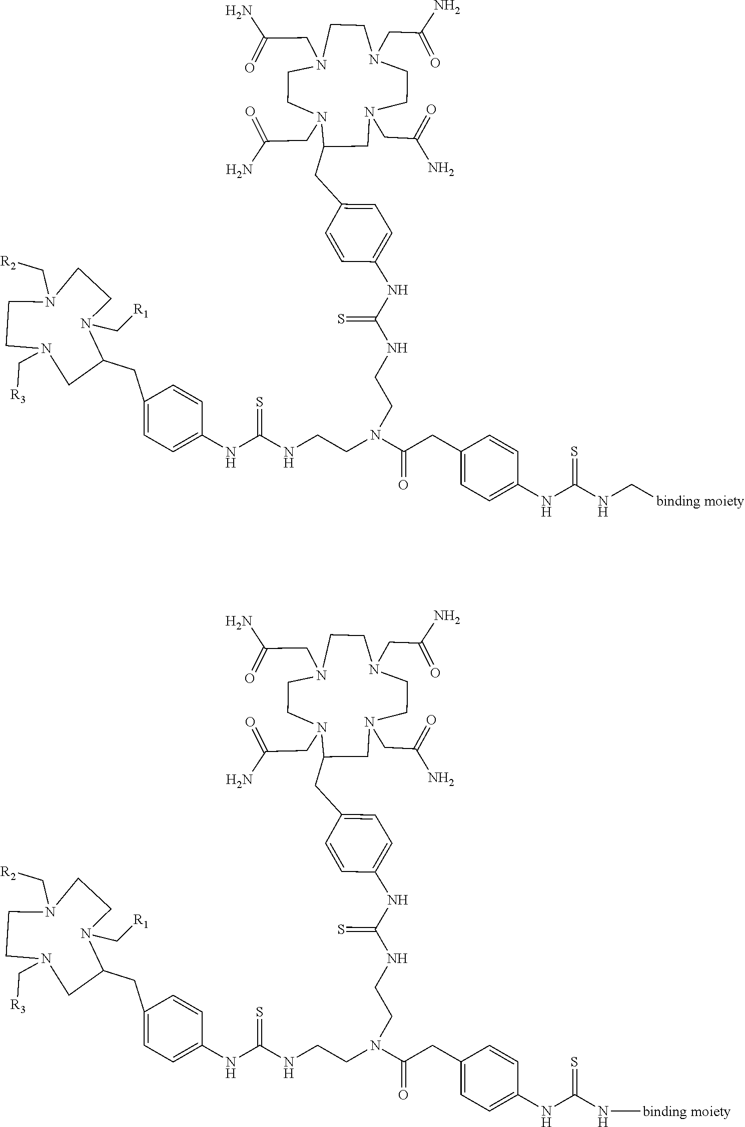

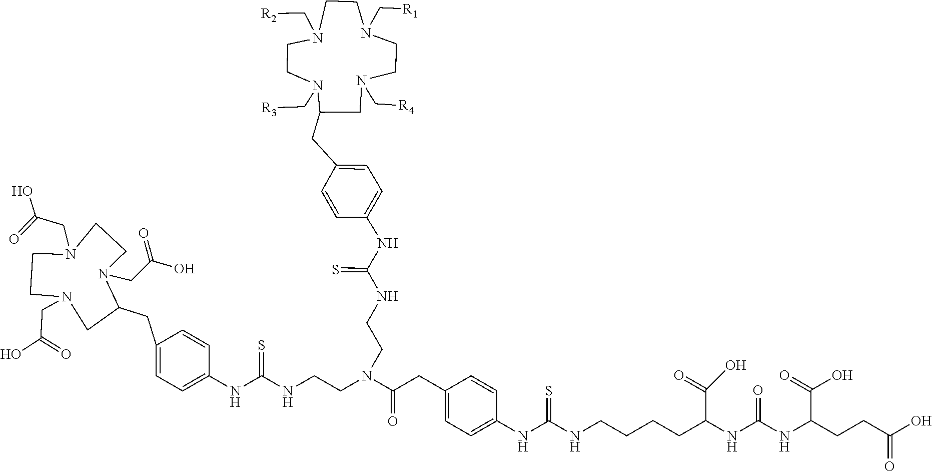

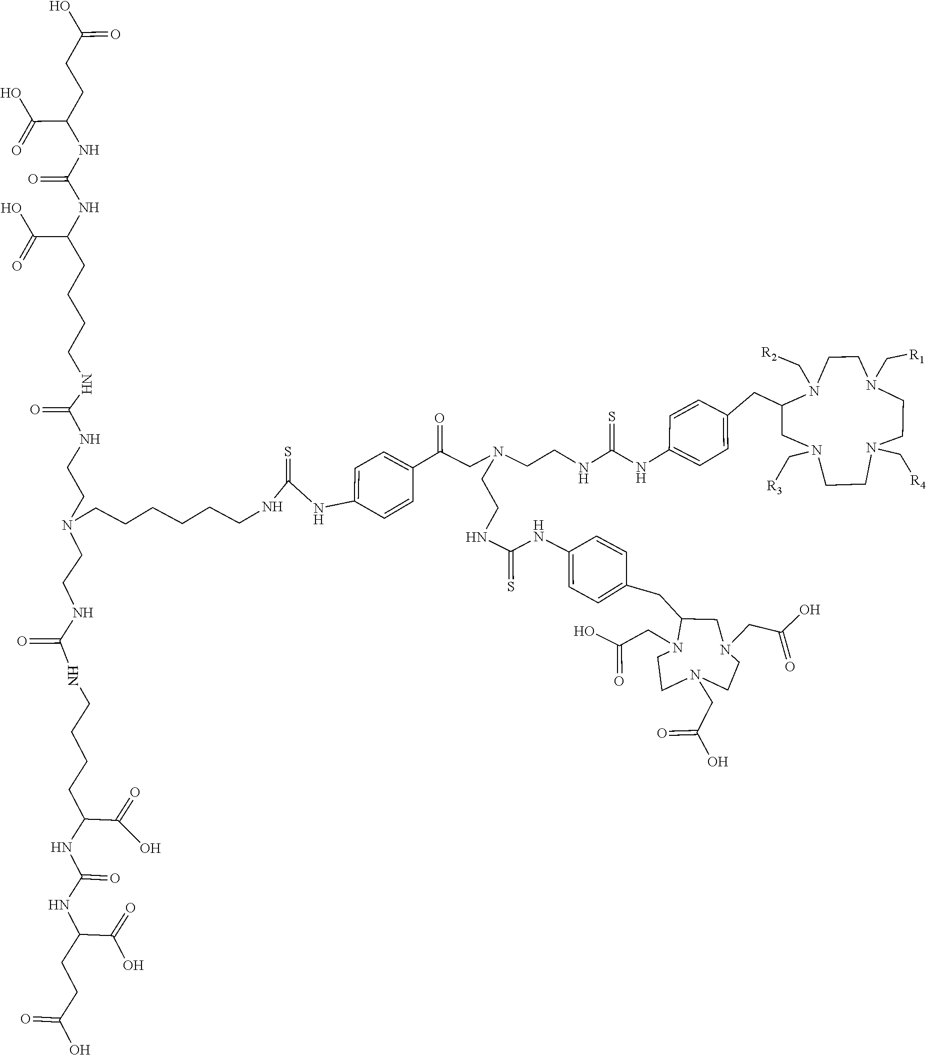

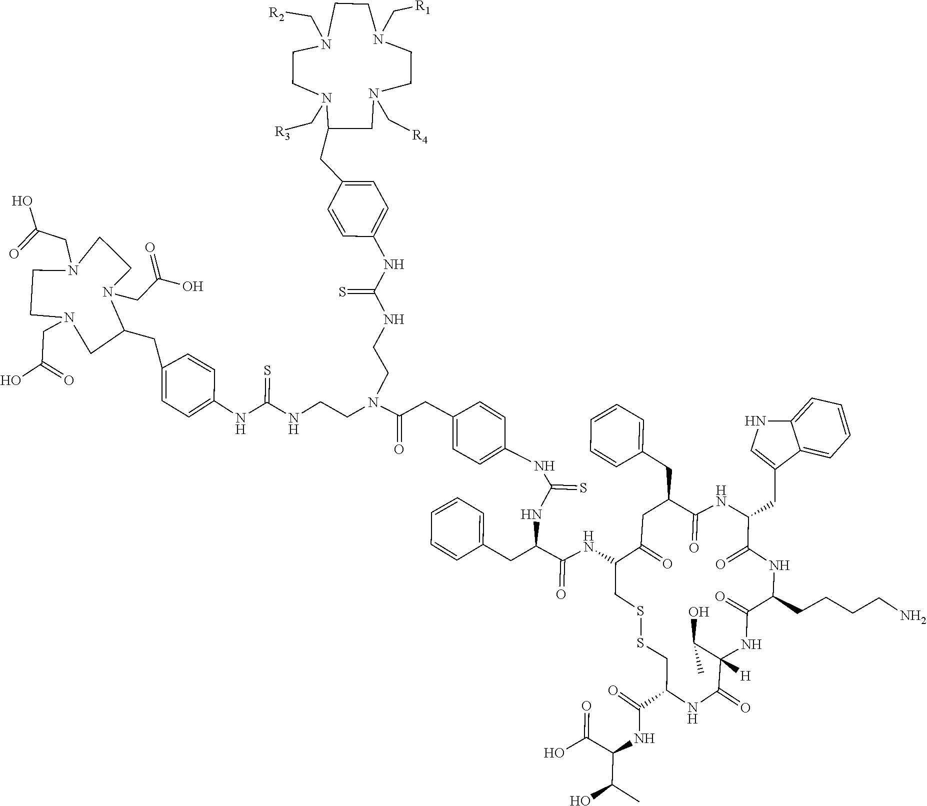

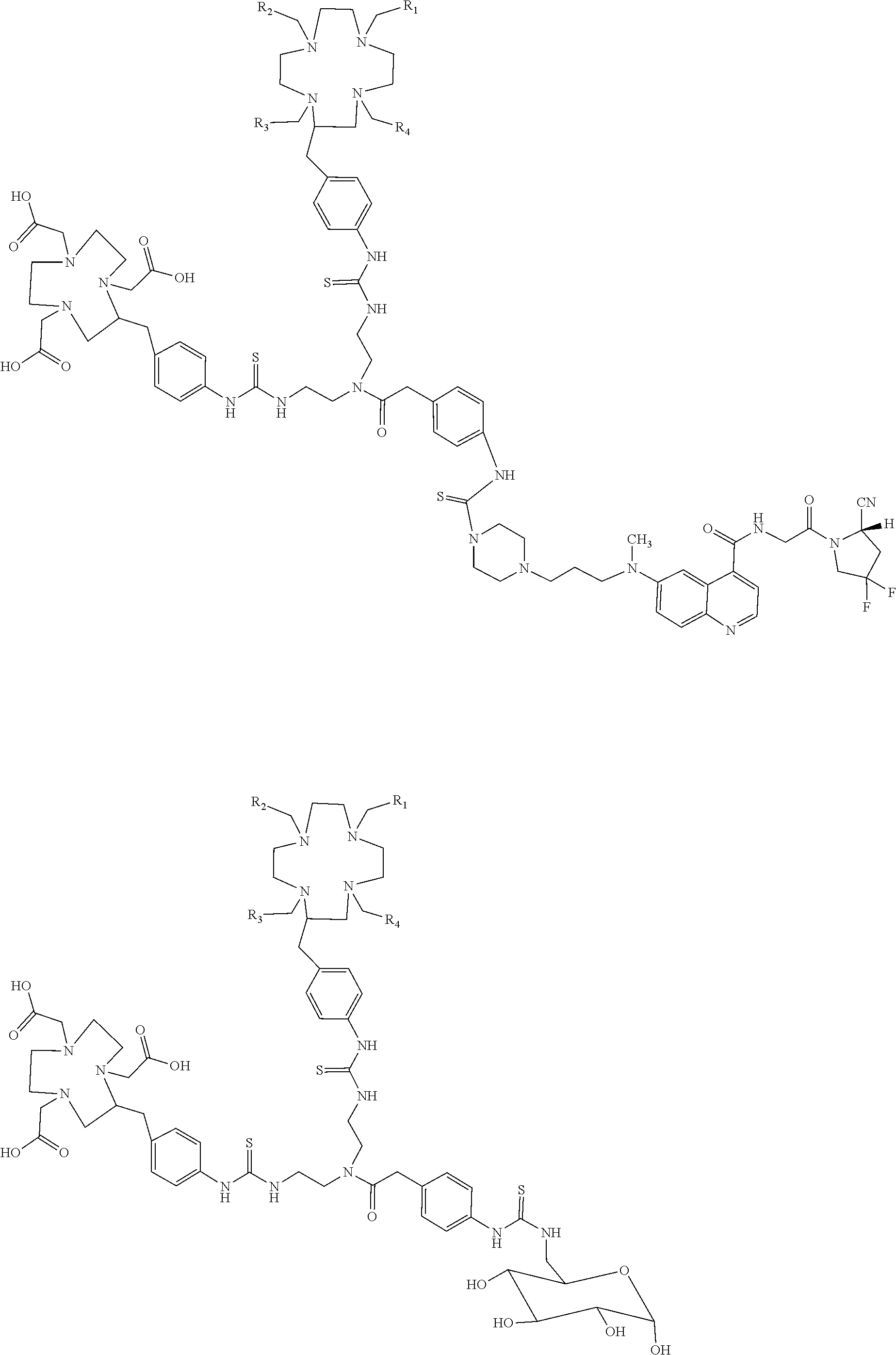

In the above conjugates, R¹, R², and R³ are each independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and at least one of R¹, R², and R³ is other than C(═O)NH₂ or C(═O)OH.

In the above conjugates, at least one of R¹, R², and R³ is selected from S(═O)₂OH, —S(═O)₂NH₂. —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂.

In the above conjugates, R¹, R², R³, and R⁴ are each independently selected from C(═O)NH₂, C(═O)OH, —S(═O)₂OH, —S(═O)₂NH₂. —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and at least one of R¹, R², R³, and R⁴ is other than C(═O)NH₂ or C(═O)OH.

In the above conjugates, at least one of R¹, R², R³, and R⁴ is selected from —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂.

In one example, R¹ is S(═O)₂OH. In one example, R¹ is —S(═O)₂NH₂. In one example, R¹ is —OP(═O)(OH)₂. In one example, R¹ is —P(═O)(OH)₂. In one example, R¹ is —OP(═O)(OH)NH₂. In one example, R¹ is C(═O)NHOH. In one example, R¹ is —C(═S)SH.

In one example, R¹ is —C(═O)SH. In one example, R¹ is C(═S)OH. In one example, R¹ is C(═S)NH₂.

In one example, R² is S(═O)₂OH. In one example, R² is —S(═O)₂NH₂. In one example, R² is —OP(═O)(OH)₂. In one example, R² is —P(═O)(OH)₂. In one example, R² is —OP(═O)(OH)NH₂. In one example, R² is C(═O)NHOH. In one example, R² is —C(═S)SH.

In one example, R² is —C(═O)SH. In one example, R² is C(═S)OH. In one example, R² is C(═S)NH₂.

In one example, R³ is S(═O)₂OH. In one example, R³ is —S(═O)₂NH₂. In one example, R³ is —OP(═O)(OH)₂. In one example, R³ is —P(═O)(OH)₂. In one example, R³ is —OP(═O)(OH)NH₂. In one example, R³ is C(═O)NHOH. In one example, R³ is —C(═S)SH.

In one example, R³ is —C(═O)SH. In one example, R³ is C(═S)OH. In one example, R³ is C(═S)NH₂.

In one example, R⁴ is S(═O)₂OH. In one example, R⁴ is —S(═O)₂NH₂. In one example, R⁴ is —OP(═O)(OH)₂. In one example, R⁴ is —P(═O)(OH)₂. In one example, R⁴ is —OP(═O)(OH)NH₂. In one example, R⁴ is C(═O)NHOH. In one example, R⁴ is —C(═S)SH. In one example, R⁴ is —C(═O)SH. In one example, R⁴ is C(═S)OH. In one example, R⁴ is C(═S)NH₂.

Additional examples of conjugates include:

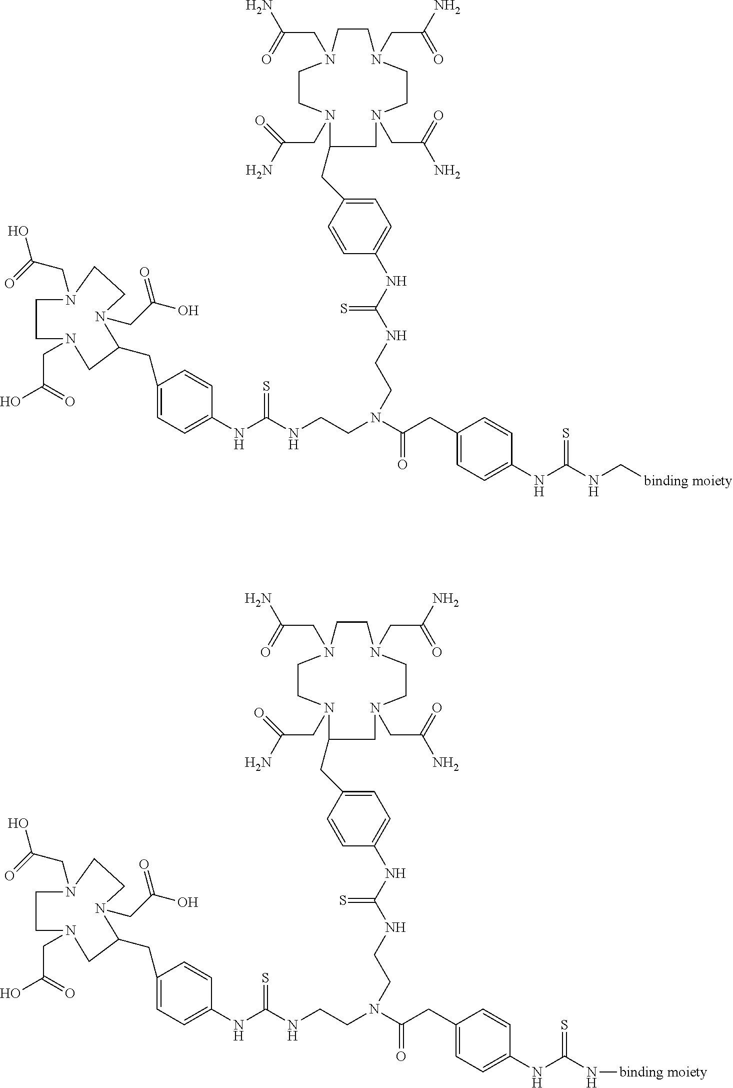

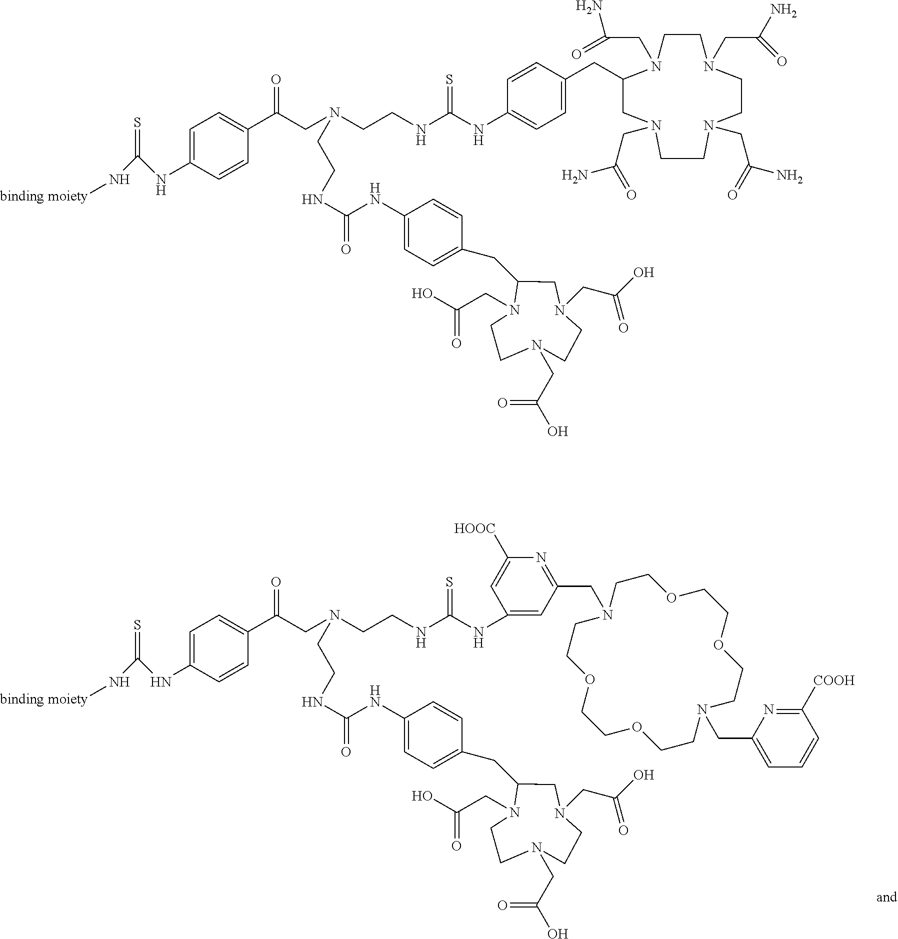

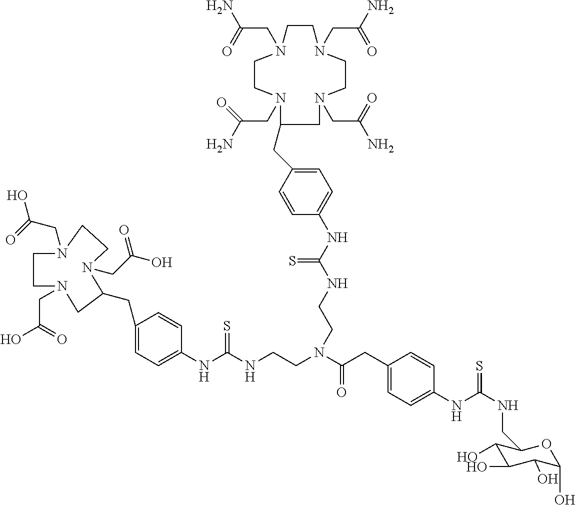 [SMILES: NC(=O)CN1CCN(CC(N)=O)CCN(CC(N)=O)C(Cc2ccc(NC(=S)NCCN(CCNC(=S)Nc3ccc(CC4CN(CC(=O)O)CCN(CC(=O)O)CCN4CC(=O)O)cc3)C(=O)Cc3ccc(NC(=S)NCC4OC(O)C(O)C(O)C4O)cc3)cc2)CN(CC(N)=O)CC1]

In some embodiments, an exemplary conjugate comprises an imaging isotope complexed to the chelator of the imaging isotope and and/or a radiotherapy isotope complexed to the chelator of the radiotherapy isotope. In some embodiments, the conjugate has the structure

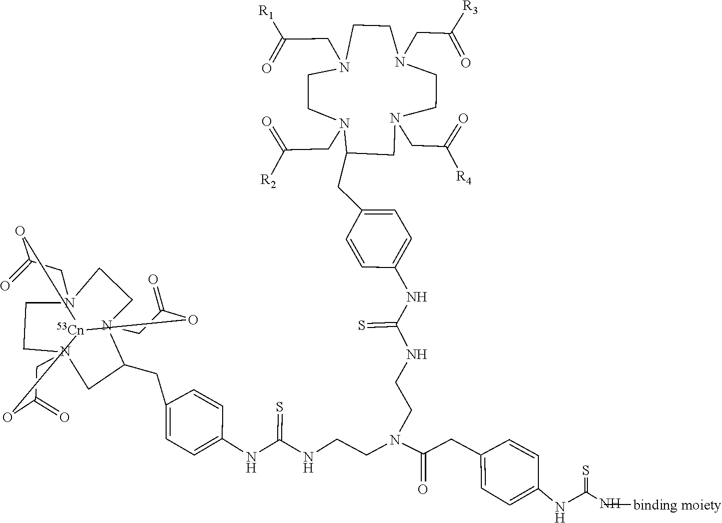

The binding moieties (targeting vectors) in the conjugates herein may be selected from any of polypeptides, antibodies, small molecules (e.g., glucose or a derivative thereof), extracellular vesicles (e.g., an exosome or a liposome), viruses, and nucleic acids as described herein.

In some embodiments, the binding moiety is an adeno-associated virus. In some embodiments, the adeno-associated virus is AAV9.

In some embodiments, the binding moiety is an extracellular vesicle. In some embodiments, the extracellular vesicle is an extracellular vesicle derived from plasma.

Below is an example of a glucose derivative usable for targeting small metastasized tumors:

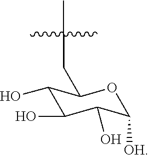

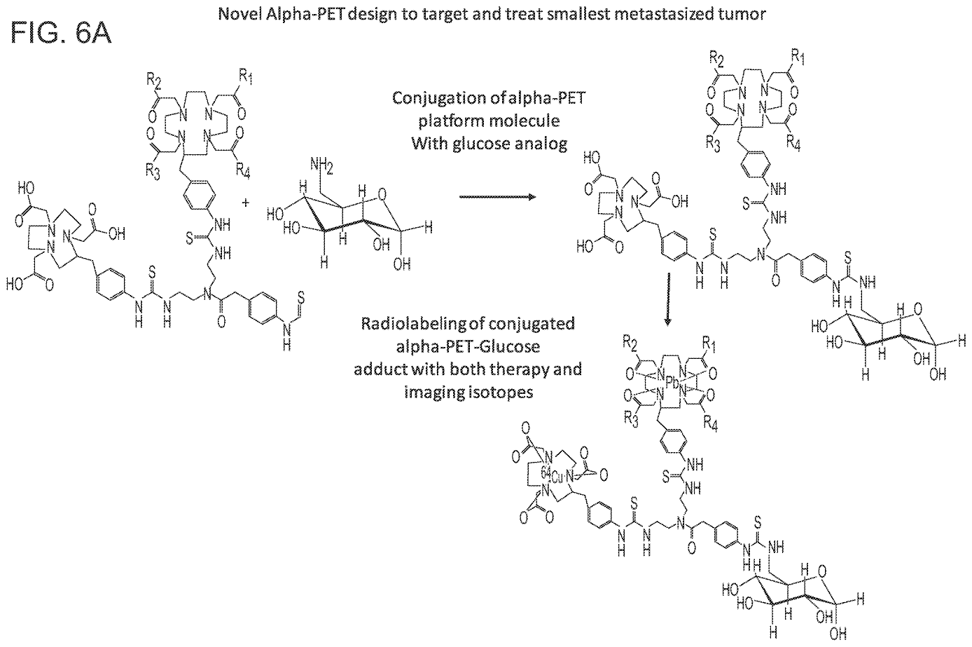

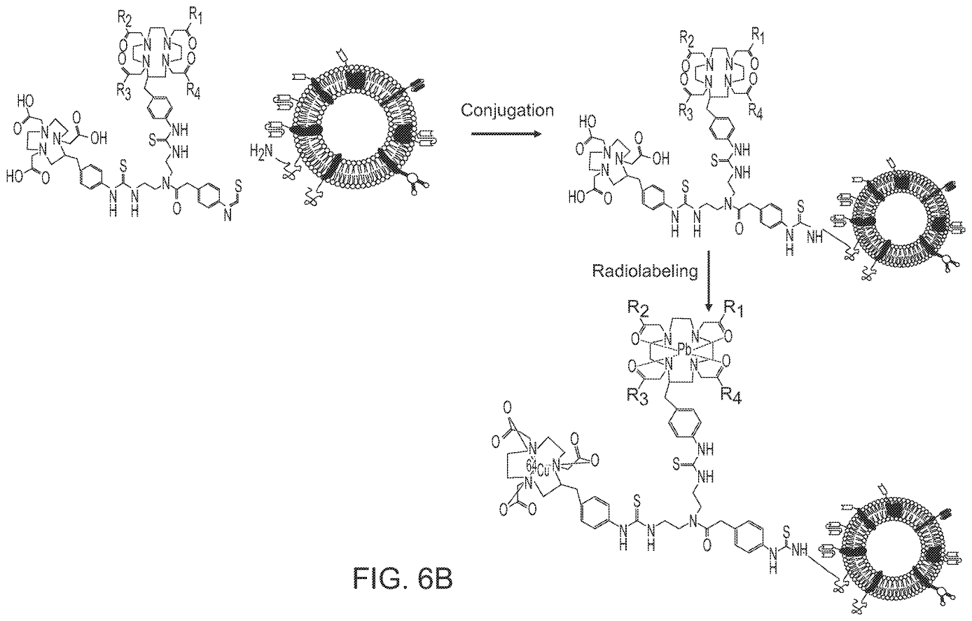

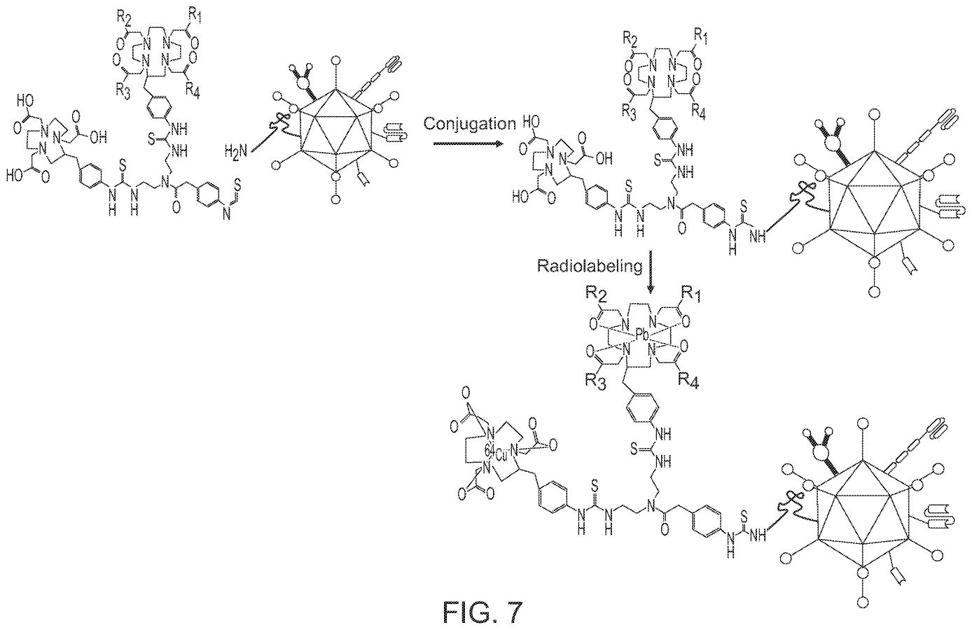

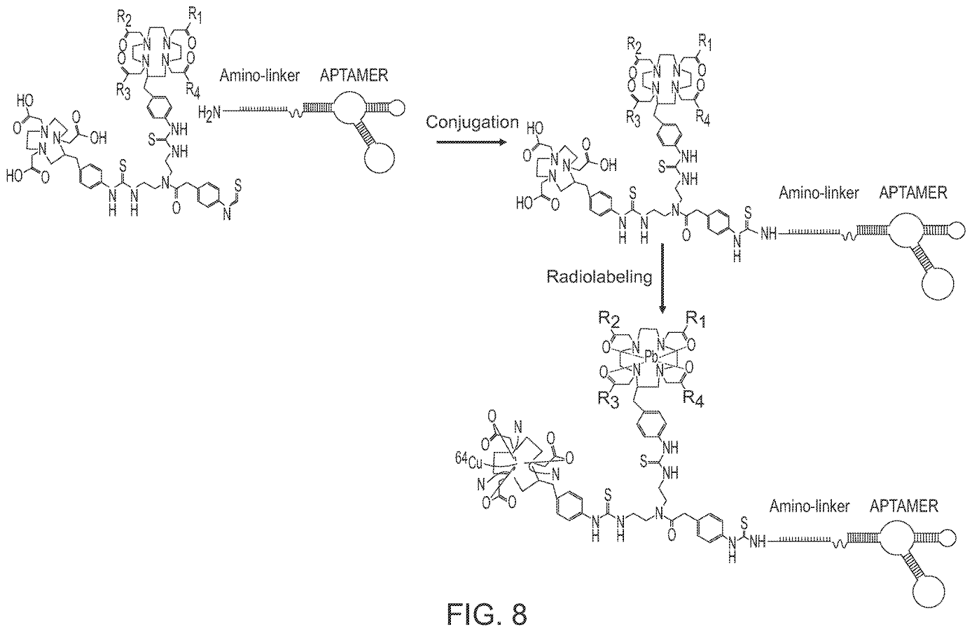

FIGS. 6B, 7, and 8 describe conjugation reactions of Alpha-PET platform moiety with extracellular vesicles, different types of viruses, and aptamers. In one example, these reactions are performed by conjugating a pre-radiolabeled Alpha-PET moiety (either labeled with imaging or therapeutic isotope or both) with a biologic. In another example, these conjugation reactions are performed without pre-radiolabeling of the alpha-PET platform moiety, followed by radiolabeling of the resulting conjugate with an imaging and/or a radiotherapy (as may be necessary). In one example of the conjugation reaction, the isothiocyanate group present on the Alpha-PET platform moiety reacts with a primary amine reactive group present on extracellular vesicle, virus, or aptamers. To facilitate the conjugation reaction, the biologic reactant molecule can be modified with linker containing an amino groups. The conjugation reaction connects the PET platform moiety and the biologic by a stable covalent thiourea bond. The biologics in the conjugates shown in FIGS. 6B, 7, and 8 are targeting vectors allowing to image and treat various diseases including but not limited to different types of cancers. The exemplary conjugates are shown in FIGS. 6B, 7, and 8 for illustrative purposes only. Other biologics described herein can be conjugated to an Alpha-PET platform moiety using methods and procedures similar to those described in those Figures.

Below table provides amino acid sequences of exemplary binding moieties (targeting vectors) that can be used in the conjugates described herein.

### Exemplary Anti-PSMA Antibody Sequences

| SEQ ID NO: | | |
|---|---|---|
| | ANTIBODY 1 | |
| 393 | VL | DIQMTQSPSSLSASVGDSVTITCRASQSINNYLNWYQQKP |
| | | GKAPKLLIYTASSLLSGVPSRFSGSGSGTDFTLTISGLHPE |
| | | DFATYFCQQSFSTPPITFGQGTRLDIK |
| 394 | VH | EVQLVESGGALVQTGGSLRLSCVASGFTFSNYWMSWVR |
| | | QSPGKGLQWVASIKKDGSDEDY |
| | | VDSVKGRFTISRDNAENSLYLQMTSLRIEDTAVYYCARFI |
| | | SAVGVDWGQGALVTVSS |
| 1 | LC CDR1 | QSINNY |
| 2 | LC CDR2 | TAS |
| 3 | LC CDR3 | QQSFSTPPIT |
| 395 | HC CDR1 | GFTFSNYW |
| 396 | HC CDR2 | IKKDGSDE |
| 397 | HC CDR3 | ARFISAVGVD |
| | ANTIBODY 2 | |
| 398 | VL | DIQMTQSPSSLSASVGDRVTITCRASQGIRNDLGWYQQK |
| | | PGRAPKRLIYGASNLQSGVPSRFSGSGSGTEFTLTISSLQP |
| | | EDFATYYCLQHNSHPYTFGQGTKLEIK |
| 399 | VH | QVQLVESGGGVVQPGRSLRLSCAASGFTFITYGMHWVR |
| | | QAPGKGLEWVAIIYYDESNKYY |
| | | ADSVKGRFTISRDISKNTLYLQMNGLRAEDTAVYYCARA |
| | | PRVAVEEDYSYYYGMDVWGQGTTVTVSS |
| 49 | LC CDR1 | QGIRND |
| 59 | LC CDR2 | GAS |
| 68 | LC CDR3 | LQHNSHPYT |
| 77 | HC CDR1 | GFTFITYG |
| 91 | HC CDR2 | IYYDESNK |
| 400 | HC CDR3 | ARAPRVAVEEDYSYYYGMDV |
| | ANTIBODY 3 | |
| 401 | VL | DIVMTQSHKFMSTSVGDRVSIICKASQDVGTAVDWYQQ |
| | | KPGQSPKLLIYWASTRHTGVPDRFTGSGSGTDFTLAITNV |
| | | QSEDLADYFCQQYNSYPLTFGAGTKLEIKR |
| 402 | VH | EVQLQQSGPELVKPGTSVRISCKTSGYTFTEYTIHWVKQS |
| | | HGKSLEWIGNINPNNGGTTY |
| | | NQKFEDKATLTVDKSSSTAYMELRSLTSEDSAVYYCAA |
| | | GWNFDYWGQGTTLT |
| 54 | LC CDR1 | KASQDVGTAVD |
| 63 | LC CDR2 | WASTRHT |
| 75 | LC CDR3 | QQYNSYPLT |
| 403 | HC CDR1 | EYTIH |
| 101 | HC CDR2 | NINPNNGGTTYNQKFED |
| 118 | HC CDR3 | GWNFDY |
| | ANTIBODY 4 | |
| 404 | VL | EIVLTQSPITMAAFLGERITITCSASSSISSNYLHWYQQKP |
| | | GFSPKLLIYRTSNLASGVPIRFSGSGSGTSYSLTIGTMEAE |
| | | DVATYYCQQGSYIPFTFGSGTKLEIKR |
| 405 | VH | QVQLQESGGGLVKPGGSLKLSCAASGFTFSDFYMYWVR |
| | | QTPEKRLEWVATISDGGGYTSYPDSVKGRFTISRDIAKNN |
| | | LYLQMNSLKSEDTAKYYCARGLWLRDALDYWGQGTSV |
| | | TVSS |
| 50 | LC CDR1 | SASSSISSNYLH |
| 60 | LC CDR2 | RTSNLAS |
| 69 | LC CDR3 | QQGSYIPFT |
| 81 | HC CDR1 | GFTFSDFYMY |
| 406 | HC CDR2 | TISDGGGYTSYPDSVK |
| 113 | HC CDR3 | GLWLRDALDY |
| | ANTIBODY 5 | |
| 407 | VL | NIVMTQFPKSMSISVGERVTLTCKASENVGTYVSWYQQ |
| | | KPEQSPKMLIYGASNRFTGVPDRFTGSGSATDFILTISSVQ |
| | | TEDLVDYYCGQSYTFPYTFGGGTKLEMK |
| 408 | VH | EVKLEESGGGLVQPGGSMKLSCVASGFTFSNYWMNWV |
| | | RQSPEKGLEWVAEIRSQSNNFATHYAESVKGRVIISRDDS |
| | | KSSVYLQMNNLRAEDTGIYYCTRRWNNFWGQGTTLTVS |
| | | S |
| 55 | LC CDR1 | KASENVGTYVS |
| 64 | LC CDR2 | GASNRFT |
| 73 | LC CDR3 | GQSYTFPYT |
| 87 | HC CDR1 | GFTFSNYWMN |
| 102 | HC CDR2 | EIRSQSNNFATHYAESVKG |
| 119 | HC CDR3 | RWNNF |
| | ANTIBODY 6 | |
| 409 | VL | DIVLTQSPASLAVSLGQRATISCRASESIDSYDNTFMHWY |
| | | QQKPGQPPNLLIFRASILESGIPARFSGSGSGTDFTLTIYPV |
| | | EADDVATYYCHQSIEDPYTFGGGTKLEIK |
| 410 | VH | EVQLQQSGPELVKPGASVKMSCKASGYTFTGYVMHWV |
| | | KQKPGQVLEWIGYINPYNDVTRYNGKFKGKATLTSDKY |
| | | SSTAYMELSGLTSEDSAVYYCARGENWYYFDSWGRGAT |
| | | LTVSS |
| 56 | LC CDR1 | RASESIDSYDNTFMH |
| 65 | LC CDR2 | RASILES |
| 74 | LC CDR3 | HQSIEDPYT |
| 411 | HC CDR1 | GYTFTGYVMH |
| 103 | HC CDR2 | YINPYNDVTRYNGKFKG |
| 120 | HC CDR3 | GENWYYFDS |
| | ANTIBODY 7 | |
| 412 | VL | NIVMTQSQKFMSTSPGDRVRVTCKASQNVGSDVAWYQ |
| | | AKPGQSPRILIYSTSYRYSGVPDRFTAYGSGTDFTLTITNV |
| | | QSEDLTEYFCQQYNSYPLTFGAGTKLELK |
| 413 | VH | QVQLKESGPGLVASSQSLSITCTVSGFSLTAYGINWVRQP |
| | | PGKGLEWLGVIWPDGNTDYNSTLKSRLNIFKDNSKNQVF |
| | | LKMSSFQTDDTARYFCARDSYGNFKRGWFDFWGQGTTL |
| | | TVSS |
| 57 | LC CDR1 | KASQNVGSDVA |
| 66 | LC CDR2 | STSYRYS |
| 75 | LC CDR3 | QQYNSYPLT |
| 89 | HC CDR1 | GFSLTAYGIN |
| 414 | HC CDR2 | VIWPDGNTDYNSTLKS |
| 121 | HC CDR3 | DSYGNFKRGWFDF |

### Exemplary Anti-Somatostatin Antibody Sequences

| SEQ ID No: | | |
|---|---|---|
| | ANTIBODY 1 | |
| 415 | VL | DIVMTQSPDSLAVSLGERATINCKSSQSLLNSRNRKNYLA |
| | | WYQQKPDQSPKLLIYWASTRESGVPDRFSGSGSGTDFTL |
| | | TISSLQAEDVAVYYCKQSYYLWTFGGGTKVEIK |
| 416 | VH | EVQLVESGGGLVQPGGSLRLSCAASGFTFSDYGMAWFR |
| | | QAPGKGLEWVSFISNLGYSIYYADSVKGRFTISRDNAKN |
| | | SLYLQMNSLRAEDTAVYYCARAPYDYDSFDPMDYWGQ |
| | | GTLVTVS |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |
| | ANTIBODY 2 | |
| 417 | VL | DIVMTQSPDSLAVSLGERATINCKSSQSLLNSRTRKNYLA |
| | | WYQQKPDQSPKLLIYWASTRESGVPDRFSGSGSGTDFTL |
| | | TISSLQAEDVAVYYCKQSYYLWTFGGGTKVEIK |
| 418 | VH | EVQLVESGGGLVQPGGSLRLSCAASGFTFSDYGMAWFR |
| | | QAPGKGPEWVSFISNLAYSIYYADSVKGRFTISRDNAKNS |
| | | LYLQMNSLRAEDTAVYYCARAPYDYDSFYPMDYWGQG |
| | | TLVTVSS |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |
| | ANTIBODY 3 | |
| 419 | VL | DIVMTQSPDSLAVSLGERATINCKSSQSLLNSRTRKNYLA |
| | | WYQQKPDQSPKLLIYWASTRASGVPDRFSGSGSGTDFTL |
| | | TISSLQAEDVAVYYCKQSYYLWTFGGGTKVEIK |
| 420 | VH | EVQLVESGGGLVQPGGSLRLSCAASGFTFSDYGMAWFR |
| | | QAPGKGPEWVSFISNLAYSIYYADSVKGRFTISRDNAKNS |
| | | LYLQMNSLRAEDTAVYYCARAPYDYDSFDPMDYWGQG |
| | | TLVTVSS |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |
| | ANTIBODY 4 | |
| 421 | VL | EVQLVESGGGLVQPGGSLRLSCAASGFTFSDVGMAWFR |
| | | QAPGKGPEWVSFISNLAVSIVVADSVKGRFTISRDNAKNS |
| | | LVLQMNSLRAEDTAVVVCARAPVDYDSFDPMDYWGQG |
| | | TLVTVSS |
| 422 | VH | DIVMTQSPDSLAVSLGERATINCKSSQSLLNSRNRKSYLA |
| | | WYQQKPDQSPKLLIYYASTRASGVPDRFSGSGSGTDFTL |
| | | TISSLQAEDVAVYYCKQSYYLWTFGGGTKVEIK |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |
| | ANTIBODY 5 | |
| 423 | VL | EVQLVESGGGLVQPGGSLRLSCAASGFTFSDYGMAWFR |
| | | QAPGKGPEWVSFISNLAYSIYYADSVKGRFTISRDNAKNS |
| | | LYLQMNSLRAEDTAVYYCARAPYDVDSFDPMDYWGQG |
| | | TLVTVSS |
| 424 | VH | DIVMTQSPDSLAVSLGERATINCKSSQSLLNSRNRKSYLA |
| | | WYQQKPDQSPKLLIYVASTRASGVPDRFSGSGSGTDFTL |
| | | TISSLQAEDVAVYYCKQSYYLWTFGGGTKVEIK |
| 123 | LC CDR1 | KSSQSLLNSRNRKNYLA |
| 125 | LC CDR2 | WASTRES |
| 128 | LC CDR3 | KQSYYLWT |
| 130 | HC CDR1 | DYGMA |
| 132 | HC CDR2 | FISNLGYSIYYADSVKG |
| 135 | HC CDR3 | APYDYDSFDPMDY |
| | ANTIBODY 6 | |
| 425 | VL | DVVMTQTPLSLPVSLGDQASISCRSSQSLVHSNGNTYLH |
| | | WYLQRPGQSPKLLIYKVSNRFSGVPDRFSGSGSGTDFTL |
| | | KISRVEAEDLGVYFCSQSTHVPFTFGSGTKLEIK |
| 426 | VH | EVKLVESGGGLVQPGGSLRLSCATSGFTFTDYHLNWVR |
| | | QPPGKALEWLALIRNKRYGYRTEYSASVKGRFTISRDNS |
| | | QSILYLQMNTLRAEDSATYYCARDFYDPFAYWGQGTLV |
| | | TVSA |
| 7 | LC CDR1 | RSSQSLVHSNGNTYLH |
| 8 | LC CDR2 | KVSNRFS |
| 9 | LC CDR3 | SQSTHVPFT |
| 10 | HC CDR1 | DYHLN |
| 427 | HC CDR2 | IRNKRYGYRTEYSASV |
| 12 | HC CDR3 | DFYDPFAY |
| | ANTIBODY 7 | |
| 428 | VL | DVVMTQTPLSLPVSLGDQASISCRSSQSLVHSNGNTYLH |
| | | WYLQRPGQSPKLLIYKVSNRFSGVPDRFSGSGSGTDFTL |
| | | KISRVEAEDLGVYFCSQSTHVPFTFGSGTKLEIK |
| 429 | VH | EVKLVESGGGLVQPGGSLRLSCATSGFTFTDYHLNWVR |
| | | QPPGKALEWLALIRNKRYGYRTEYSASVKGRFTISRDNS |
| | | QSILYLQMNTLRAEDSATYYCARDFYDPFAYWGQGTLV |
| | | TVSA |
| 7 | LC CDR1 | RSSQSLVHSNGNTYLH |
| 8 | LC CDR2 | KVSNRFS |
| 9 | LC CDR3 | SQSTHVPFT |
| 10 | HC CDR1 | DYHLN |
| 11 | HC CDR2 | IRNKRYGYRTEYSASVKG |
| 12 | HC CDR3 | DFYDPFAY |
| | ANTIBODY 8 | |
| 430 | VL | DVVMTQTPLSLPVSLGDQASISCRSSQSLVHSNGNTYLH |
| | | WYLQRPGQSPNLLIYKVSNRFSGVPDRFSGSGSGTDFTL |
| | | KISRVEAEDLGLYFCSQSTRVPFTFGSGTKLEIK |
| 431 | VH | EVKLVESGGGLVQPGGSLRLSCATSGFTFTDYHMNWVR |
| | | QPPGKALEWLALIRNKANGYRTEYSASVKGRFTISRDNS |
| | | QNILYLQMNTLRAEDSATYYCARDFYDPFAYWGQGTLV |
| | | TVSA |
| 7 | LC CDR1 | RSSQSLVHSNGNTYLH |
| 8 | LC CDR2 | KVSNRFS |
| 127 | LC CDR3 | SQSTRVPFT |
| 129 | HC CDR1 | DYHMN |
| 131 | HC CDR2 | LIRNKANGYRTEYSASVKG |
| 12 | HC CDR3 | DFYDPFAY |

### Exemplary Anti-FAP Antibody Sequences

| SEQ ID | | |
|---|---|---|
| NO: | | |
| | ANTIBODY 1 | |
| 432 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYKFPYTFGQGTKLEI |
| 433 | VH | EVQIIESGGGIVQPGGSLRLSCAASGFTFSSYAMSWVRQA |
| | | PGKGIEWVSAISGSGGTTYYADSVKGRFTISRDNSKNTL |
| | | YIQMNSLRAEDTAVYYCAKDAGRPYFDYWGQGTLVTV |
| | | SS |
| 13 | LC CDR1 | RASQSISSYLN |
| 14 | LC CDR2 | AASSLQS |
| 434 | LC CDR3 | QQSYKFPYT |
| 16 | HC CDR1 | SYAMS |
| 17 | HC CDR2 | AISGSGGTTYYADSVKG |
| 435 | HC CDR3 | DAGRPYFD |
| | ANTIBODY 2 | |
| 436 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPRTFGQGTKLEIK |
| 437 | VH | EVQLLESGGGLVQPGGSLRLSCAASGFTFSSYAMSWVRQ |
| | | APGKGLEWVSAISGSGGGTRYADSVKGRFTISRDNSKNT |
| | | LYLQMNSLRAEDTAVYYCAKHSSGFHWYFDYWGQGTL |
| | | VTVSS |
| 438 | LC CDR1 | RASQSISSYL |
| 14 | LC CDR2 | AASSLQS |
| 143 | LC CDR3 | QQSYSTPRT |
| 16 | HC CDR1 | SYAMS |
| 152 | HC CDR2 | AISGSGGGTRYADSVKG |
| 157 | HC CDR3 | HSSGFHWYFDY |
| | ANTIBODY 3 | |
| 439 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSSPYTFGQGTKLEIK |
| 440 | VH | EVQLLESGGGLVQPGGSLRLSCAASGFTFSSYAMSWVRQ |
| | | APGKGLEWVSGISGSGGSTYYADSVKGRFTISRDNSKNT |
| | | LYLQMNSLRAEDTAVYYCAKISFYPGGTYFDYWGQGTL |
| | | VTVSS |
| 13 | LC CDR1 | RASQSISSYLN |
| 14 | LC CDR2 | AASSLQS |
| 441 | LC CDR3 | QQSYSSPYT |
| 16 | HC CDR1 | SYAMS |
| 442 | HC CDR2 | GISGSGGSTYYADSVKG |
| 158 | HC CDR3 | ISFYPGGTYFDY |
| | ANTIBODY 4 | |
| 443 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPYTFGQGTKLEIK |
| 444 | VH | EVQLLESGGGLVQPGGSLRLSCAASGFTFSSYAMSWVRQ |
| | | APGKGLEWVSGISGSGGSTYYADSVKGRFTISRDNSKNT |
| | | LYLQMNSLRAEDTAVYYCAKISFYPGGTYFDYWGQGTL |
| | | VTVSS |
| 13 | LC CDR1 | RASQSISSYLN |
| 14 | LC CDR2 | AASSLQS |
| 445 | LC CDR3 | QQSYSTPYT |
| 16 | HC CDR1 | SYAMS |
| 442 | HC CDR2 | GISGSGGSTYYADSVKG |
| 158 | HC CDR3 | ISFYPGGTYFDY |
| | ANTIBODY 5 | |
| 446 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYVCQQSYSTPYTFGQGTKLEIK |
| 447 | VH | EVQLLESGGGLVQPGGSLRLSCAASGFTFSSYAMNWVR |
| | | QAPGKGLEWVSTISSSGSRTYYADSVKGRFTISRDNSKNT |
| | | LYLQMNSLRAEDTAVYVCAKGLVASAPFDYWGQGTLV |
| | | TVSS |
| 13 | LC CDR1 | RASQSISSYLN |
| 14 | LC CDR2 | AASSLQS |
| 445 | LC CDR3 | QQSYSTPYT |
| 149 | HC CDR1 | SYAMN |
| 448 | HC CDR2 | TISSSGSRTYYADSVKG |
| 159 | HC CDR3 | GLVASAPFD |
| | ANTIBODY 6 | |
| 449 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPYTFGQGTKLEIK |
| 450 | VH | EVQLLESGGGLVQPGGSLRLSCAASGFTFSSYAMSWVRQ |
| | | APGKGLEWVSGISGGGGSTYYADSVKGRFTISRDNSKNT |
| | | LYLQMNSLRAEDTAVYYCAKIAHSRIGWHFDYWGQGTL |
| | | VTVSS |
| 13 | LC CDR1 | RASQSISSYLN |
| 14 | LC CDR2 | AASSLQS |
| 445 | LC CDR3 | QQSYSTPYT |
| 16 | HC CDR1 | SYAMS |
| 155 | HC CDR2 | GISGGGGSTYYADSVKG |
| 160 | HC CDR3 | IAHSRIGWHFDY |
| | ANTIBODY 7 | |
| 451 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPYTFGQGTKLEIK |
| 452 | VH | EVQLLESGGGLVQPGGSLRLSCAASGFTFSRYAMTWVR |
| | | QAPGKGLEWVSSISASGGSTYYADSVKGRFTISRDNSKN |
| | | TLYLQMNSLRAEDTAVYVCAKTFSGYAHYDFDYWGQG |
| | | TLVTVSS |
| 13 | LC CDR1 | RASQSISSYLN |
| 14 | LC CDR2 | AASSLQS |
| 445 | LC CDR3 | QQSYSTPYT |
| 151 | HC CDR1 | RYAM |
| 453 | HC CDR2 | SISASGGSTYYADSVKG |
| 161 | HC CDR3 | TFSGYAHYDFDY |
| | ANTIBODY 8 | |
| 454 | VL | EIVLTQSPGTLSLSPGERATLSCRASQSVSRNYLAWYQQ |
| | | KPGQAPRLLIYGASSRATGIPDRFSGSGSGTDFTLTISRLE |
| | | PEDFAVYYCQQSLGYPPTFGQGTKVEIK |
| 455 | VH | EVQLLESGGGLVQPGGSLRLSCAASGFTFSPAYMSWVRQ |
| | | APGKGLEWVSAISGSGGSTYYADSVKGRFTISRDNSKNT |
| | | LYLQMNSLRAEDTAVYYCAKGWRAFDYWGQGTLVTVS |
| | | S |
| 137 | LC CDR1 | RASQSVSRNYLA |
| 140 | LC CDR2 | GASSRAT |
| 145 | LC CDR3 | QQSLGYPPT |
| 456 | HC CDR1 | GFTFSPAYMS |
| 457 | HC CDR2 | AISGSGGSTYYADSVK |
| 458 | HC CDR3 | GWRAFDY |

### Exemplary Anti-CD3 Antibody Sequences

| SEQ ID NO: | | |
|---|---|---|
| | ANTIBODY 1 | |
| 459 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSNWPPLTFGGGTKVEIK |
| 460 | VH | QVQLVESGGGVVQPGRSLRLSCAASGFKFSGYGMHWVR |
| | | QAPGKGLEWVAVIWYDGSKKYYVDSVKGRFTISRDNSK |
| | | NTLYLQMNSLRAEDTAVYYCARQMGYWHFDLWGRGT |
| | | LVTVSS |
| 19 | LC CDR1 | RASQSVSSYLA |
| 20 | LC CDR2 | DASNRAT |
| 21 | LC CDR3 | QQRSNWPPLT |
| 22 | HC CDR1 | GYGMH |
| 461 | HC CDR2 | VIWYDGSKKYYVDSVKGR |
| 24 | HC CDR3 | QMGYWHFDL |
| | ANTIBODY 2 | |
| 461 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSNWPPLTFGGGTKVEIK |
| 462 | VH | QVQLVQSGGGVVQSGRSLRLSCAASGFKFSGYGMHWV |
| | | RQAPGKGLEWVAVIWYDGSKKYYVDSVKGRFTISRDNS |
| | | KNTLYLQMNSLRGEDTAVYYCARQMGYWHFDLWGRG |
| | | TLVTVSS |
| 19 | LC CDR1 | RASQSVSSYLA |
| 20 | LC CDR2 | DASNRAT |
| 21 | LC CDR3 | QQRSNWPPLT |
| 22 | HC CDR1 | GYGMH |
| 462 | HC CDR2 | IWYDGSKKYYVDSVKG |
| 24 | HC CDR3 | QMGYWHFDL |
| | ANTIBODY 3 | |
| 463 | VL | EIVLTQSPATLSLSPGERATLSCRASQSVSSYLAWYQQKP |
| | | GQAPRLLIYDASNRATGIPA |
| | | RFSGSGSGTDFTLTISSLEPEDFAVYYCQQRSNWPWTFG |
| | | QGTKVEIK |
| 464 | VH | QVQLVESGGGVVQPGRSLRLSCAASGFTFRSYGMHWVR |
| | | QAPGKGLEWVAIIWYDGSKKNYADSVKGRFTISRDNSK |
| | | NTLYLQMNSLRAEDTAVYYCARGTGYNWFDPWGQGTL |
| | | VTVSS |
| 19 | LC CDR1 | RASQSVSSYLA |
| 20 | LC CDR2 | DASNRAT |
| 170 | LC CDR3 | QQRSNWPWT |
| 172 | HC CDR1 | SYGMH |
| 174 | HC CDR2 | AIWYNGRKQDYADSVKG |
| 175 | HC CDR3 | GTGYNWFDP |
| | ANTIBODY 4 | |
| 465 | VL | QTVVTQEPSLTVSPGGTVTLTCGSSTGAVTSGYYPNWVQ |
| | | QKPGQAPRGLIGGTKFLAPGTPARFSGSLLGGKAALTLS |
| | | GVQPEDEAEYYCALWYSNRWVFGGGTKLTVL |
| 466 | VH | EVQLVESGGGLVQPGGSLKLSCAASGFTFNIYAMNWVR |
| | | QAPGKGLEWVARIRSKYNNYATYYADSVKSRFTISRDDS |
| | | KNTAYLQMNNLKTEDTAVYYCVRHGNFGNSYVSFFAY |
| | | WGQGTLVTVSS |
| 467 | LC CDR1 | GSSTGAVTSGYYPN |
| 468 | LC CDR2 | GTKFLAP |
| 469 | LC CDR3 | ALWYSNRWV |
| 470 | HC CDR1 | IYAMN |
| 471 | HC CDR2 | RIRSKYNNYATYYADSVKS |
| 472 | HC CDR3 | HGNFGNSYVSFFAY |
| | ANTIBODY 5 | |
| 473 | VL | QTVVTQEPSLTVSPGGTVTLTCGSSTGAVTSGYYPNWVQ |
| | | QKPGQAPRGLIGGTKFLAPGTPARFSGSLLGGKAALTLS |
| | | GVQPEDEAEYYCALWYSNRWVFGGGTKLTVL |
| 474 | VH | EVQLVESGGGLVQPGGSLKLSCAASGFTFNKYAMNWVR |
| | | QAPGKGLEWVARIRSKYNNYATYYADSVKDRFTISRDD |
| | | SKNTAYLQMNNLKTEDTAVYYCVRHGNFGNSYISYWA |
| | | YWGQGTLVTVSS |
| 467 | LC CDR1 | GSSTGAVTSGYYPN |
| 468 | LC CDR2 | GTKFLAP |
| 469 | LC CDR3 | ALWYSNRWV |
| 475 | HC CDR1 | KYAMN |
| 471 | HC CDR2 | RIRSKYNNYATYYADSVKS |
| 472 | HC CDR3 | HGNFGNSYISYWAY |
| | ANTIBODY 6 | |
| 473 | VL | QTVVTQEPSLTVSPGGTVTLTCGSSTGAVTSGYYPNWVQ |
| | | QKPGQAPRGLIGGTKFLAPGTPARFSGSLLGGKAALTLS |
| | | GVQPEDEAEYYCALWYSNRWVFGGGTKLTVL |
| 474 | VH | EVQLVESGGGLEQPGGSLKLSCAASGFTFNSYAMNWVR |
| | | QAPGKGLEWVARIRSKYNNYATYYADSVKGRFTISRDD |
| | | SKNTAYLQMNNLKTEDTAVYYCVRHGNFGNSYLSFWA |
| | | YWGQGTLVTVSS |
| 467 | LC CDR1 | GSSTGAVTSGYYPN |
| 468 | LC CDR2 | GTKFLAP |
| 469 | LC CDR3 | ALWYSNRWV |
| 149 | HC CDR1 | SYAMN |
| 475 | HC CDR2 | RIRSKYNNYATYYADSVKG |
| 476 | HC CDR3 | HGNFGNSYLSFWAY |
| | ANTIBODY 7 | |
| 477 | VL | QTVVTQEPSLTVSPGGTVTLTCGSSTGAVTSGYYPNWVQ |
| | | QKPGQAPRGLIGGTKFLAPGTPARFSGSLLGGKAALTLS |
| | | GVQPEDEAEYYCALWYSNRWVFGGGTKLTVL |
| 478 | VH | EVQLVESGGGLVQPGGSLKLSCAASGFTFNRYAMNWVR |
| | | QAPGKGLEWVARIRSKYNNYATYYADSVKGRFTISRDD |
| | | SKNTAYLQMNNLKTEDTAVYYCVRHGNFGNSYLSYFA |
| | | YWGQGTLVTVSS |
| 467 | LC CDR1 | GSSTGAVTSGYYPN |
| 468 | LC CDR2 | GTKFLAP |
| 469 | LC CDR3 | ALWYSNRWV |
| 479 | HC CDR1 | RYAMN |
| 480 | HC CDR2 | RIRSKYNNYATYYADS |
| 481 | HC CDR3 | HGNFGNSYLSYFAY |

### Exemplary Anti-CD20 Antibody Sequences

| SEQ ID | | |
|---|---|---|
| NO: | | |
| | ANTIBODY 1 | |
| 482 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSNWPLTFGGGTKVEIK |
| 483 | VH | AVQLVESGGGLVQPGRSLRLSCAASGFTFGDYTMHWVR |
| | | QAPGKGLEWVSGISWNSGSIGYADSVKGRFTISRDNAKN |
| | | SLYLQMNSLRAEDTALYYCTKDNQYGSGSTYGLGVWG |
| | | QGTLVTVSS |
| 19 | LC CDR1 | RASQSVSSYLA |
| 20 | LC CDR2 | DASNRAT |
| 484 | LC CDR3 | QQRSNWPLT |
| 485 | HC CDR1 | DYTMH |
| 486 | HC CDR2 | GISWNSGSILGYADSVKG |
| 487 | HC CDR3 | DNQYGSGSTYGLGV |
| | ANTIBODY 2 | |
| 488 | VL | DIQMTQSPSSLSASVGDRVTITCRASQGISSWLAWYQQK |
| | | PEKAPKSLIYAASSLQSGVPSRFSGSGSGTDFTLTISSLQPE |
| | | DFATYYCQQYNSVFTFGPGTKVDIK |
| 489 | VH | AVQLVESGGGLVQPGRSLRLSCAASGFTFGDYTMHWVR |
| | | QAPGKGLEWVSGISWNSGSIGYADSVKGRFTISRDNAKN |
| | | SLYLQMNSLRAEDTALYYCTKDNQYGSGSTYGLGVWG |
| | | QGTLVTVSS |
| 490 | LC CDR1 | RASQGISSWLA |
| 14 | LC CDR2 | AASSLQS |
| 491 | LC CDR3 | QQYNSVFT |
| 485 | HC CDR1 | DYTMH |
| 486 | HC CDR2 | GISWNSGSILGYADSVKG |
| 487 | HC CDR3 | DNQYGSGSTYGLGV |
| | ANTIBODY 3 | |
| 492 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSDWPLTFGGGTKVEIK |
| 493 | VH | AVQLVESGGGLVQPGRSLRLSCAASGFTFGDYTMHWVR |
| | | QAPGKGLEWVSGISWNSGSIGYADSVKGRFTISRDNAKN |
| | | SLYLQMNSLRAEDTALYYCTKDNQYGSGSTYGLGVWG |
| | | QGTLVTVSS |
| 19 | LC CDR1 | RASQSVSSYLA |
| 20 | LC CDR2 | DASNRAT |
| 494 | LC CDR3 | QQRSDWPLT |
| 485 | HC CDR1 | DYTMH |
| 486 | HC CDR2 | GISWNSGSILGYADSVKG |
| 487 | HC CDR3 | DNQYGSGSTYGLGV |
| | ANTIBODY 4 | |
| 495 | VL | QIVLSQSPAILSASPYEKVTMTCRASSSVSYIHWFQQKPG |
| | | SSPKPWIYATSNLASGVPVRFSGSGSGTSYSLTISRVEAED |
| | | AATYYCQQWTSNPPTFGGGTKLEIK |
| 496 | VH | QVQLQQPGAELVKPGASVKMSCKASGYTFTSYNMHWV |
| | | KQTPGRGLEWIGAIYPGNGDTSYNQKFKGKATLTADKSS |
| | | STAYMQLSSLTSEDSAVYYCARSTYYGGDWYFNVWGT |
| | | TVTVSA |
| 25 | LC CDR1 | RASSSVSYIH |
| 26 | LC CDR2 | ATSNLAS |
| 27 | LC CDR3 | QQWTSNPPT |
| 28 | HC CDR1 | SYNMH |
| 29 | HC CDR2 | AIYPGNGDTSYNQKFKG |
| 192 | HC CDR3 | STYYGGDWYFNV |

### Exemplary Anti-CXCR4 Antibody Sequences

| SEQ ID | | |
|---|---|---|
| NO: | | |
| | ANTIBODY 1 | |
| 497 | VL | DIVMTQSPDSLAVSLGERATINCKSSQSLFNSRTRKNYLA |
| | | WYQQKPGQPPKLLIYWASTRESGVPDRFSGSGSGTDFTL |
| | | TISSLQAEDVAVYYCMQSFNLRTFGQGTKVEIK |
| 498 | VH | EVQLVESGGGLVQPGRSLRLSCTASGFTFTDNYMSWVR |
| | | QAPGKGLEWVGFIRNKANGYTT |
| | | EYAASVKGRFTISRDDSKSIAYLQMNSLKTEDTAVYYCA |
| | | RDVGSNYFDYWGQGTLVTVSS |
| 499 | LC CDR1 | NSLFNSRTRKNY |
| 32 | LC CDR2 | WAS |
| 211 | LC CDR3 | MQSFNLRT |
| 221 | HC CDR1 | GFTFTDNY |
| 35 | HC CDR2 | IRNKANGYTT |
| 268 | HC CDR3 | ARDVGSNYFDY |
| | ANTIBODY 2 | |
| 500 | VL | DIVMTQSPSSLAVSLGERATMSCKSSQSLFNSRTRKNYL |
| | | AWYQQKPGQSPKLLIYWASARDSGVPARFTGSGSETYFT |
| | | LTISRVQAEDLAVYYCMQSFNLRTFGQGTKVEIK |
| 501 | VH | EVNLVESGGGLVQPGRSLRLSCTASGFTFTDNYMSWVR |
| | | QAPGKGLEWLGFIRNKANGYTTDYAASVRGRFTISRDNS |
| | | KSILYLQMNALRTEDTAVYYCARDVGSNYFDYWGQGT |
| | | LVTVSS |
| 499 | LC CDR1 | NSLFNSRTRKNY |
| 32 | LC CDR2 | WAS |
| 211 | LC CDR3 | MQSFNLRT |
| 221 | HC CDR1 | GFTFTDNY |
| 35 | HC CDR2 | IRNKANGYTT |
| 268 | HC CDR3 | ARDVGSNYFDY |
| | ANTIBODY 3 | |
| 502 | VL | DIVMSQSPSSLAVSAGEKVTMSCKSSQSLFNSRTRKNYL |
| | | AWYQQKPGQSPKLLIYWASARDSGVPARFTGSGSETYFT |
| | | LTISRVQAEDLAVYYCMQSFNLRTFGGGTKLEIK |
| 503 | VH | EVNLVESGGGLVQPGGSLRLSCATSGFTFTDNYMSWVR |
| | | QPPGKALEWLGFIRNKANGYTTDYSASVRGRFTISRDNS |
| | | QSILYLQMNALRAEDSATYYCARDVGSNYFDYWGQGTT |
| | | LTVSS |
| 504 | LC CDR1 | QSLFNSRTRKNY |
| 32 | LC CDR2 | WAS |
| 211 | LC CDR3 | MQSFNLRT |
| 221 | HC CDR1 | GFTFTDNY |
| 35 | HC CDR2 | IRNKANGYTT |
| 268 | HC CDR3 | ARDVGSNYFDY |
| | ANTIBODY 4 | |
| 505 | VL | SSELTQDPAVSVALGQTVRITCQGDSLRKFFASWYQQKP |
| | | GQAPVLVIYGKNSRPSGIPDRFSGSNSRNTASLTITGAQA |
| | | EDEGDYYCNSRDSRDNHQVFGAGTKVTVLS |
| 506 | VH | QVQLVQSGAEVKKPGASVKVSCKASGYTFTSYGISWVR |
| | | QAPGQGLEWMGWISAYNGNTNYAQKLQGRVTMTTDTS |
| | | TSTAYMELRSLRSDDTAVYYCARDTPGIAARRYYYYGM |
| | | DVWGQGTTVTVSS |
| 197 | LC CDR1 | QGDSLRKFFAS |
| 206 | LC CDR2 | GKNSRPS |
| 215 | LC CDR3 | NSRDSRDNHQV |
| 227 | HC CDR1 | SYGIS |
| 249 | HC CDR2 | WISAYNGNTNYAQKLQG |
| 274 | HC CDR3 | DTPGIAARRYYYYGMDV |
| | ANTIBODY 5 | |
| 507 | VL | QPVLTQPRSVSGSPGQSVTISCTGTISDVGGHNFVSWYQQ |
| | | NPGKAPKLIIFEVTKRPAGVPDRFSGSKSGNTASLTVSGL |
| | | QAEDEGEYYCSSYGGSNDVIFGGGTKLSVLG |
| 508 | VH | EVQLVESGGGVVQPGRSLRLSCAASGFTFSSYGMHWVR |
| | | QAPGKGLEWVAVISYDGSNKYYADSVKGRFTISRDNSK |
| | | NTLYLQVSSLRAEDTAVYYCVRDLVAAAGTAFDIWGQG |
| | | TTVTVSS |
| 195 | LC CDR1 | TGTISDVGGHNFVS |
| 204 | LC CDR2 | EVTKRPA |
| 213 | LC CDR3 | SSYGGSNDVI |
| 225 | HC CDR1 | SYGMH |
| 247 | HC CDR2 | VISYDGSNKYYADSVKG |
| 272 | HC CDR3 | DLVAAAGTAFDI |
| | ANTIBODY 6 | |
| 509 | VL | QSVLTQPPSVSKGLRQTATLTCTGNSNNVGNQGAAWLQ |
| | | QHQGHPPKLLSYRNNNRPSGIS |
| | | ERFSASRSGNTASLTITGLQPEDEADYYCSAWDNRLKTY |
| | | VFGTGGKVTVLG |
| 510 | VH | QVQLQQSGPGLVKPSQTLSLTCAVSGDSVSSNFVAWNWI |
| | | RQSPSRGLEWLGRTYYRSRWY |
| | | NDYAVSVQSRIRVTPDTSKNQFSLHLDSVTPEDTAVYYC |
| | | ARGQHSGFDFWGQGTLVTVSS |
| 196 | LC CDR1 | TGNSNNVQNQGAA |
| 205 | LC CDR2 | RNNNRPS |
| 214 | LC CDR3 | SAWDNRLKTYV |
| 226 | HC CDR1 | SNFVAWN |
| 248 | HC CDR2 | RTYYRSRWYNDYAVSVQS |
| 273 | HC CDR3 | GQHSGFDF |
| | ANTIBODY 7 | |
| 511 | VL | EIVLTQSPATLSVSPGERATLSCRASQSVNTNLAWYQQK |
| | | PGQAPRLLIYGASSRATGIPDRFSGSGSGTDFTLTISRLEPE |
| | | DFAVYYCQHYGSSPLTFGGGTKLEIKR |
| 512 | VH | QVQLVQSGGGVVQPGRSLRLSCAASGFTFSSYPMHWVR |
| | | QAPGKGLEWMTVISSDGRNKYYPDSVKGRFTISRDNSKN |
| | | TLYLQMNSLRPEDTAVYYCARGGYHDFWSGPDYWGQG |
| | | TLVTVSS |
| 198 | LC CDRI | RASQSVNTNLA |
| 140 | LC CDR2 | GASSRAT |
| 216 | LC CDR3 | QHYGSSPLT |
| 228 | HC CDR1 | SYPMH |
| 250 | HC CDR2 | VISSDGRNKYYPDSVKG |
| 275 | HC CDR3 | GGYHDFWSGPDY |
| | ANTIBODY 8 | |
| 513 | VL | SSELTQDPAVSVALGQTVRITCQGDSLRSYYASWYQQKP |
| | | GQAPVLVIYGKNNRPSGIPDRFSGSKSHNTAYLTITGAQA |
| | | EDEADYFCNSRSGSQRVFGGGTKLTVLG |
| 524 | VH | EVQLVQSGGGLVQPGGSLRLSCAASGFTFSSYAMSWVR |
| | | QAPGKGLEWVANIKQDGSEKYYVDSVKGRFTISRDNAK |
| | | NSLYLQMNSLRAEDTAVYYCARDQVSGITIFGGKWRSP |
| | | DVWGQGTTVTVSS |
| 199 | LC CDR1 | QGDSLRSYYAS |
| 208 | LC CDR2 | GKNNRPS |
| 217 | LC CDR3 | NSRSGSQRV |
| 231 | HC CDR1 | SYAMS |
| 251 | HC CDR2 | NIKQDGSEKYYVDSVKG |
| 276 | HC CDR3 | DQVSGITIFGGKWRSPDV |
| | ANTIBODY 9 | |
| 525 | VL | QSVLTQPPSASGTPGQRVTISCSGSRSNIGSNTVNWYQQL |
| | | PGTAPKLLIYTNNQRPSGVPDRFSGSKSGTSASLAITGLQ |
| | | AEDEADYYCLSFDSSLTSYVFGTGTKVTVLG |
| 526 | VH | QVTLKESGGGVVQPGRSLRLSCAASGFTFTNYGLHWVR |
| | | QAPGKGLEWVAVISHDGTKKYYADSVKGRFTISRDSSEN |
| | | TLYLQMNSLRPEDSALYYCARDGGYCSGGRCYSYGMD |
| | | VWGQGTTVTVSS |
| 200 | LC CDR1 | SGSRSNIGSNTVN |
| 209 | LC CDR2 | TNNQRPS |
| 218 | LC CDR3 | LSFDSSLTSYV |
| 229 | HC CDR1 | NYGLH |
| 252 | HC CDR2 | VISHDGTKKYYADSVKG |
| 277 | HC CDR3 | DGGYCSGGRCYSYGMDV |
| | ANTIBODY 10 | |
| 527 | VL | QSVLTQPPSASGTPGQRVTISCSGSRSNIGGNTVNWYQQL |
| | | PGTAPKLLIYANNQRPSGVP |
| | | DRFSGSKSGTSASLAISGLRSEDEADYYCAAWDDNLSGH |
| | | VVFGGGTKLTVLR |
| 528 | VH | QVQLVQSGGGVVLPGRSLRLSCVASGFTFRRYGMHWVR |
| | | QAPGKGLEWVSLISYDGSKTFYGESVKGRFTISRDNSKN |
| | | TLYLQMNSLRAEDTAVYYCARATVTTDGYYYMDVWG |
| | | KGTTVTVSS |
| 201 | LC CDR1 | SGSRSNIGGNTVN |
| 210 | LC CDR2 | ANNQRPS |
| 529 | LC CDR3 | AAWDDNLSGHVV |
| 230 | HC CDR1 | RYGMH |
| 253 | HC CDR2 | LISYDGSKTFYGESVKG |
| 278 | HC CDR3 | ATVTTDQYYYMDV |

### Exemplary Anti-HER2 Antibody Sequences

| SEQ ID | | |
|---|---|---|
| NO: | | |
| | ANTIBODY 1 | |
| 530 | VL | DIQMTQSPSSLSASVGDRVTITCRASQDVNTAVAYQQKP |
| | | GKAPKLLIYSASFLYSGVPSRFSGSRSGTDFTLTISSLQPE |
| | | DFATYYCQQHYTTPPTFGQGTKVEIKRT |
| 531 | VH | EVQLVESGGGLVQPGGSLRLSCAASGFNIKDTYIHWVRQ |
| | | APGKGLEWVARIYPTNGYTRYADSVKGRFTISADTSKNT |
| | | AYLQMNSLRAEDTAVYYCSRWGGDGFYAMDYWGQGT |
| | | LVTVSSA |
| 295 | LC CDR1 | RASQDVNTAVA |
| 304 | LC CDR2 | SASFLYS |
| 532 | LC CDR3 | QQHYTTPPT |
| 309 | HC CDR1 | GFNIKDTYIH |
| 533 | HC CDR2 | RIYPTNGYTRYADSVKG |
| 316 | HC CDR3 | WGGDGFYAMDY |
| | ANTIBODY 2 | |
| 534 | VL | DIQMTQSPSSLSASVGDRVTITCKASQDVSIGVAWYQQK |
| | | PGKAPKLLIYSASYRYTGVPSRFSGSGSGTDFTLTISSLQP |
| | | EDFATYYCQQYYIYPYTFGQGTKVEIK |
| 535 | VH | EVQLVESGGGLVQPGGSLRLSCAASGFTFTDYTMDWVR |
| | | QAPGKGLEWVADVNPNSGGSIYNQRFKGRFTLSVDRSK |
| | | NTLYLQMNSLRAEDTAVYYCARNLGPSFYFDYWGQGTL |
| | | VTVSS |
| 37 | LC CDR1 | KASQDVSIGVA |
| 536 | LC CDR2 | SASYRY |
| 537 | LC CDR3 | QQYYIYPY |
| 40 | HC CDR1 | GFTFTDYTMD |
| 538 | HC CDR2 | ADVNPNSGGSIYNQRFK |
| 539 | HC CDR3 | ARNLGPSFYFDY |
| | ANTIBODY 3 | |
| 540 | VL | DTVMTQSHKIMSTSVGDRVSITCKASQDVSIGVAWYQQ |
| | | RPGQSPKLLIYSASYRYTGVPDRFTGSGSGTDFTFTISSVQ |
| | | AEDLAVYYCQQYYIYPYTFGGGTKLEIK |
| 541 | VH | EVQLQQSGPELVKPGTSVKISCKASGFTFTDYTMDWVK |
| | | QSHGKSLEWIGDVNPNSGGSIYNQRFKGKASLTVDRSSRI |
| | | VYMELRSLTFEDTAVYYCARNLGPSFYFDYWGQGTTLT |
| | | VSS |
| 37 | LC CDR1 | KASQDVSIGVA |
| 384 | LC CDR2 | SASYRYT |
| 39 | LC CDR3 | QQYYIYPYT |
| 40 | HC CDR1 | GFTFTDYTMD |
| 41 | HC CDR2 | DVNPNSGGSIYNQRFKG |
| 42 | HC CDR3 | NLGPSFYFDY |
| | ANTIBODY 4 | |
| 542 | VL | DIQMTQSPSSLSASVGDRVTITCKASQDVSIGVAWYQQK |
| | | PGKAPKLLIYSASYRYTGVPSRFSGSGSGTDFTLTISSLQP |
| | | EDFATYYCQQYYIYPYTFGQGTKVEIK |
| 543 | VH | EVQLVESGGGLVQPGGSLRLSCAASGFTFTDYTMDWVR |
| | | QAPGKGLEWVADVNPNSGGSIYNQRFKGRFTLSVDRSK |
| | | NTLYLQMNSLRAEDTAVYYCARNLGPSFYFDYWGQGTL |
| | | VTVSS |
| 37 | LC CDR1 | KASQDVSIGVA |
| 384 | LC CDR2 | SASYRYT |
| 39 | LC CDR3 | QQYYIYPYT |
| 40 | HC CDR1 | GFTFTDYTMD |
| 41 | HC CDR2 | DVNPNSGGSIYNQRFKG |
| 42 | HC CDR3 | NLGPSFYFDY |
| | ANTIBODY 5 | |
| 544 | VL | DIQMTQSPSSLSASVGDRVTITCRASQSISNYLAWYQQKP |
| | | GKAPKLLIYAASSLESGVPSRFSGSGSGTDFTLTISSLQPE |
| | | DFATYYCQQYNSLPWTFGQGTKVEIK |
| 545 | VH | EVQLVESGGGLVQPGGSLRLSCAASGFTFSSYAMSWVR |
| | | QAPGKGLEWVAVISGDGGSTYYADSVKGRFTISRDNSK |
| | | NTLYLQMNSLRAEDTAVYYCARGRVGYSLYDYWGQGT |
| | | LVTVSS |
| 546 | LC CDR1 | RASQSISNYLA |
| 547 | LC CDR2 | AASSLES |
| 548 | LC CDR3 | QQYNSLPWT |
| 549 | HC CDR1 | GFTFSSYAMS |
| 550 | HC CDR2 | VISGDGGSTYYADSVKG |
| 551 | HC CDR3 | GRVGYSLYDY |

### Exemplary Anti-VEGF Antibody Sequences

| SEQ ID | | |
|---|---|---|
| NO: | | |
| | ANTIBODY 1 | |
| 552 | VL | DIQMTQSPSSLSASVGDRVTITCSASQDISNYLNWYQQKP |
| | | GKAPKVLIYFTSSLHSGVPSRFSGSGSGTDFTLTISSLQPE |
| | | DFATYYCQQYSTVPWTFGQGTKVEIK |
| 553 | VH | EVQLVESGGGLVQPGGSLRLSCAASGYTFTNYGMNWVR |
| | | QAPGKGLEWVGWINTYTGEPTYAADFKRRFTSLDTSKST |
| | | AYLQMNSLRAEDTAVYYCAKYPHYYGSSHWYFDVWG |
| | | QGTLVTVSS |
| 554 | LC CDR1 | QDISNY |
| 555 | LC CDR2 | FTS |
| 45 | LC CDR3 | QQYSTVPWT |
| 556 | HC CDR1 | GYTFTNYG |
| 557 | HC CDR2 | INTYTGEP |
| 558 | HC CDR3 | AKYPHYYGSSHWYFDV |
| | ANTIBODY 2 | |
| 559 | VL | DIQLTQSPSSLSASVGDRVTITCSASQDISNYLNWYQQKP |
| | | GKAPKVLIYFTSSLHSGVPSRFSGSGSGTDFTLTISSLQPE |
| | | DFATYYCQQYSTVPWTFGQGTKVEIK |
| 560 | VH | EVQLVESGGGLVQPGGSLRLSCAASGYDFTHYGMNWV |
| | | RQAPGKGLEWVGWINTYTGEPTYAADFKRRFTSLDTSK |
| | | STAYLQMNSLRAEDTAVYYCAKYPYYYGTSHWYFDVW |
| | | GQGTLVTVSS |
| 554 | LC CDR1 | QDISNY |
| 555 | LC CDR2 | FTS |
| 45 | LC CDR3 | QQYSTVPWT |
| 561 | HC CDR1 | GYDFTHYG |
| 557 | HC CDR2 | INTYTGEP |
| 562 | HC CDR3 | AKYPYYYGTSHWYFDV |
| | ANTIBODY 3 | |
| 563 | VL | EIVMTQSPSTLSASVGDRVIITCQASEIIHSWLAWYQQKP |
| | | GKAPKLLIYLASTLASGVPSRFSGSGSGAEFTLTISSLQPD |
| | | DFATYYCQNVYLASTNGANFGQGTKLTVLG |
| 564 | VH | SLRLSCTASGFSLTDYYYMTWVRQAPGKGLEWVGFIDP |
| | | DDDPYYATWAKGRFTISRDNSKNTLYLQMNSLRAEDTA |
| | | VYYCAGGDHNSGWGLDIWGQGTLVTVSS |
| 565 | LC CDR1 | QASEIIHSWLA |
| 566 | LC CDR2 | LASTLAS |
| 567 | LC CDR3 | QNVYLASTNGAN |
| 568 | HC CDR1 | GFSLTDYYYMT |
| 569 | HC CDR2 | FIDPDDDPYYATWAKG |
| 570 | HC CDR3 | GDHNSGWGLDI |
| | ANTIBODY 4 | |
| 571 | VL | EIVMTQSPSTLSASVGDRVIITCQSSQSVYGNIWMAWYQ |
| | | QKPGKAPKLLIYQASKLASGVPSRFSGSGSGAEFTLTISSL |
| | | QPDDFATYYCQGNFNTGDRYAFGQGTKLTVLG |
| 572 | VH | EVQLVESGGGLVQPGGSLRLSCAASGFTISRSYWICWVR |
| | | QAPGKGLEWVSCIYGDNDITPLYANWAKGRFTISRDNSK |
| | | NTLYLQMNSLRAEDTAVYYCAKLGYADYAYDLWGQGT |
| | | LVTVSS |
| 573 | LC CDR1 | QSSQSVYGNIWMA |
| 574 | LC CDR2 | QASKLAS |
| 575 | LC CDR3 | QGNFNTGDRYA |
| 576 | HC CDR1 | GFTISRSYWIC |
| 577 | HC CDR2 | CIYGDNDITPLYANWA |
| 578 | HC CDR3 | LGYADYAYDL |
| | ANTIBODY 5 | |
| 579 | VL | DIQMTQSPSSLSASVGDRVTITCSASQDISNYLNWYQQKP |
| | | GKAPKVLIYFTSSLHSGVPSRFSGSGSGTDFTLTISSLQPE |
| | | DFATYYCQQYSTVPWTFGQGTKVEIKRTV |
| 580 | VH | EVQLVESGGGLVQPGGSLRLSCAASGYTFTNYGMNWVR |
| | | QAPGKGLEWVGWINTYTGEPTYAADFKRRFTFSLDTSKS |
| | | TAYLQMNSLRAEDTAVYYCAKYPHYYGSSHWYFDVWG |
| | | QGTL |
| 43 | LC CDR1 | SASQDISNYLN |
| 44 | LC CDR2 | FTSSLHS |
| 45 | LC CDR3 | QQYSTVPWT |
| 46 | HC CDR1 | GYTFTNYGMN |
| 337 | HC CDR2 | WINTYTGEPTYAADFKR |
| 581 | HC CDR3 | YPHYYGSSHWYFDV |
| | ANTIBODY 6 | |
| 582 | VL | DIQLTQSPSSLSASVGDRVTITCSASQDISNYLNWYQQKP |
| | | GKAPKVLIYFTSSLHSGVPSRFSGSGSGTDFTLTISSLQPE |
| | | DFATYYCQQYSTVPWTFGQGTKVEIKRTV |
| 583 | VH | EVQLVESGGGLVQPGGSLRLSCAASGYDFTHYGMNWV |
| | | RQAPGKGLEWVGWINTYTGEPTYAADFKRRFTFSLDTS |
| | | KSTAYLQMNSLRAEDTAVYYCAKYPYYYGTSHWYFDV |
| | | WGQGTL |
| 43 | LC CDR1 | SASQDISNYLN |
| 44 | LC CDR2 | FTSSLHS |
| 45 | LC CDR3 | QQYSTVPWT |
| 332 | HC CDR1 | GYDFTHYGMN |
| 337 | HC CDR2 | WINTYTGEPTYAADFKR |
| 584 | HC CDR3 | YPYYYGTSHWYFDV |

### Exemplary Anti-PD-L1 Antibody Sequences

| SEQ ID | | |
|---|---|---|
| NO: | | |
| | ANTIBODY 1 | |
| 585 | VL | DIQMTQSPSSLSASVGDRVTITCRASQDVSTAVAWYQQK |
| | | PGKAPKLLIYSASFLYSGVPSRFSGSGSGTDFTLTISSLQPE |
| | | DFATYYCQQYLYHPATFGQGTKVEIK |
| 586 | VH | EVQLVESGGGLVQPGGSLRLSCAASGFTFSDSWIHWVRQ |
| | | APGKGLEWVAWISPYGGSTYYADSVKGRFTISADTSKNT |
| | | AYLQMNSLRAEDTAVYYCARRHWPGGFDYWGQGTLVT |
| 345 | LC CDR1 | DVSTAVA |
| 346 | LC CDR2 | SASFLY |
| 347 | LC CDR3 | QQYLYHPAT |
| 348 | HC CDR1 | DSWIH |
| 349 | HC CDR2 | WISPYGGSTY |
| 350 | HC CDR3 | RHWPGGF |
| | ANTIBODY 2 | |
| 587 | VL | EIVLTQSPGTLSLSPGERATLSCRASQRVSSSYLAWYQQK |
| | | PGQAPRLLIYDASSRATGIPDRFSGSGSGTDFTLTISRLEPE |
| | | DFAVYYCQQYGSLPWTFGQGTKVEIK |
| 588 | VH | EVQLVESGGGLVQPGGSLRLSCAASGFTFSRYWMSWVR |
| | | QAPGKGLEWVANIKQDGSEKYYVDSVKGRFTISRDNAK |
| | | NSLYLQMNSLRAEDTAVYYCAREGGWFGELAFDYWGQ |
| | | GTLVT |
| 357 | LC CDR1 | RVSSSYLA |
| 361 | LC CDR2 | DASSRA |
| 365 | LC CDR3 | QQYGSLPWT |
| 369 | HC CDR1 | RYWMS |
| 373 | HC CDR2 | NIKQDGSEKY |
| 377 | HC CDR3 | EGGWFGELAF |
| | ANTIBODY 3 | |
| 589 | VL | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQRSNWPTFGQGTKVEIK |
| 590 | VH | QVQLVQSGAEVKKPGSSVKVSCKTSGDTFSTYAISWVRQ |
| | | APGQGLEWMGGIIPIFGKAHYAQKFQGRVTITADESTST |
| | | AYMELSSLRSEDTAVYFCARKFHFVSGSPFGMDVWGQG |
| | | TTVT |
| 358 | LC CDR1 | SVSSYLA |
| 362 | LC CDR2 | DASNRA |
| 366 | LC CDR3 | QQRSNWPT |
| 370 | HC CDR1 | TYAIS |
| 374 | HC CDR2 | GIIPIFGKAH |
| 378 | HC CDR3 | KFHFVSGSPFGM |
| | ANTIBODY 4 | |
| 591 | VL | QSALTQPASVSGSPGQSITISCTGTSSDVGGYNYVSWYQ |
| | | QHPGKAPKLMIYDVSNRPSGVSNRFSGSKSGNTASLTISG |
| | | LQAEDEADYYCSSYTSSSTRVFGTGTKVTVL |
| 592 | VH | EVQLLESGGGLVQPGGSLRLSCAASGFTFSSYIMMWVRQ |
| | | APGKGLEWVSSIYPSGGITFYADTVKGRFTISRDNSKNTL |
| | | YLQMNSLRAEDTAVYYCARIKLGTVTTVDYWGQGTLV |
| | | T |
| 359 | LC CDR1 | VGGYNYVS |
| 363 | LC CDR2 | DVSNRP |
| 367 | LC CDR3 | SSYTSSSTRV |
| 371 | HC CDR1 | SYIMM |
| 375 | HC CDR2 | SIYPSGGITF |
| 379 | HC CDR3 | IKLGTVTTV |
| | ANTIBODY 5 | |
| 593 | VL | QVQLVQSGAEVKKPGSSVKVSCKASGGTFSRSAISWVRQ |
| | | APGQGLEWMGVIIPAFGEANYAQKFQGRVTITADESTST |
| | | AYMELSSLRSEDTAVYYCARGRQMFGAGIDFWGQGTLV |
| | | TVSS |
| 594 | VH | EVQLVESGGGLVQPGGSLRLSCAASGFTFSDSWIHWVRQ |
| | | APGKGLEWVAWISPYGGSTYYADSVKGRFTISADTSKNT |
| | | AYLQMNSLRAEDTAVYYCARRHWPGGFDYWGQGTLVT |
| 360 | LC CDR1 | TRSSGSIDSNYVQ |
| 364 | LC CDR2 | EDNQRPS |
| 368 | LC CDR3 | QSYDSNNRHVI |
| 372 | HC CDR1 | GTFSRSAIS |
| 376 | HC CDR2 | VIIPAFGEANYAQKFQG |
| 380 | HC CDR3 | ARGRQMFGAGIDF |

### Exemplary Anti-TROP2 Antibody Sequences

| SEQ ID | | |
|---|---|---|
| NO: | | |
| | ANTIBODY 1 | |
| 595 | VL | DIQLTQSHKFMSTSVGDRVSITCKASQDVSIAVAWYQQK |
| | | PGQSPKLLIYSASYRYTGVPDRFTGSGSGTDFTFTISSVQA |
| | | EDLAVYYCQQHYITPLTFGAGTKLELKR |
| 596 | VH | VKLQESGPELKKPGETVKISCKASGYTFTNYGMNWVKQ |
| | | APGKGLKWMGWINTYTGEPTYTDDFKGRFAFSLETSAT |
| | | TAYLQINNLKSEDMATYFCARGGFGSSYWYFDVWGQG |
| | | TTVTVSS |
| 351 | LC CDR1 | KASQDVSIAVA |
| 352 | LC CDR2 | SASYRTT |
| 353 | LC CDR3 | QQHYITPLT |
| 354 | HC CDRI | YNYGM |
| 355 | HC CDR2 | WINTYTGEPTYTDDFKG |
| 356 | HC CDR3 | GGFGSSYWYFDV |
| | ANTIBODY 2 | |
| 597 | VL | DIQMTQSPSSLSASVGDRVTITCRASQSISSYLNWYQQKP |
| | | GKAPKLLIYAASSLQSGVPSRFSGSGSGTDFTLTISSLQPE |
| | | DFATYYCQQSYSTPLTFGGGTKVEI |
| 598 | VH | VQLVQSGSELKKPGASVKVSCKASGYTFTSYAMNWVRQ |
| | | APGQGLEWMGWINTNTGNPTYAQGFTGRFVFSLDTSVS |
| | | TAYLQISSLKADDTAVYYCAREDSNGYKIFDY |
| 13 | LC CDR1 | RASQSISSYLN |
| 14 | LC CDR2 | AASSLQS |
| 385 | LC CDR3 | QQSYSTPLT |
| 387 | HC CDR1 | TSYAM |
| 389 | HC CDR2 | WINTNTGNPTYAQGFTG |
| 391 | HC CDR3 | EDSNGYKIFDY |
| | ANTIBODY 3 | |
| 599 | VL | DIQLTQSPSSLSASVGDRVSITCKASQDVSIAVAWYQQKP |
| | | GKAPKLLIYSASYRYTGVPDRFSGSGSGTDFTLTISSLQPE |
| | | DFAVYYCQQHYITPLTFGAGTKVEIKR |
| 600 | VH | QVQLQQSGSELKKPGASVKVSCKASGYTFTNYGMNWV |
| | | KQAPGQGLKWMGWINTYTGEPTYTDDFKGRFAFSLDTS |
| | | VSTAYLQISSLKADDTAVYFCARGGFGSSYWYFV |
| 351 | LC CDR1 | KASQDVSIAVA |
| 384 | LC CDR2 | SASYRYT |
| 353 | LC CDR3 | QQHYITPLT |
| 388 | HC CDR1 | TNYGM |
| 355 | HC CDR2 | WINTYTGEPTYTDDFKG |
| 356 | HC CDR3 | GGFGSSYWYFDV |

### Example 1

### Radiosynthesis and Preclinical Evaluation of [⁶⁴Cu]Cu-NOTA-TCMC-AAVs

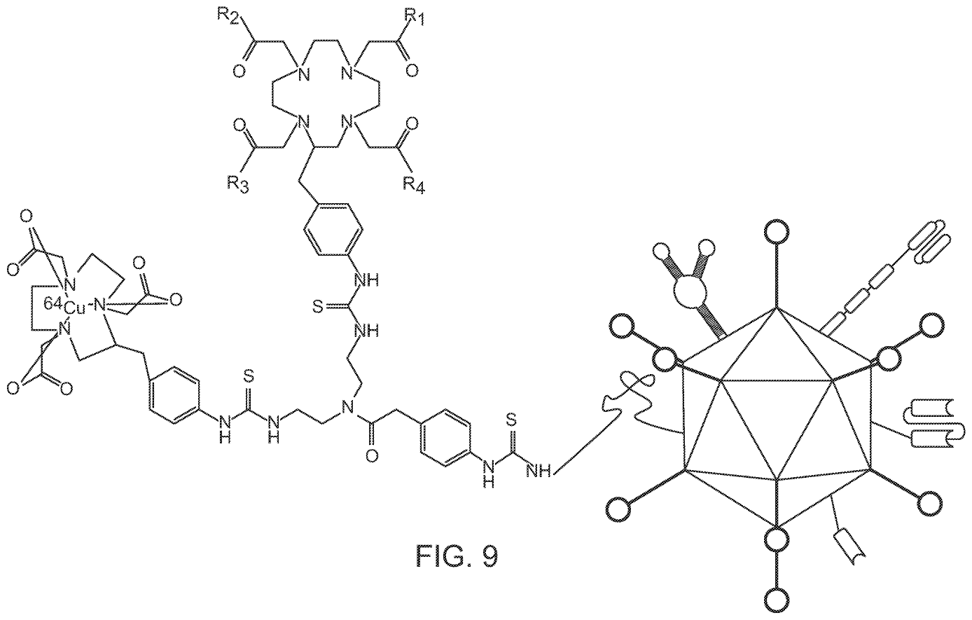

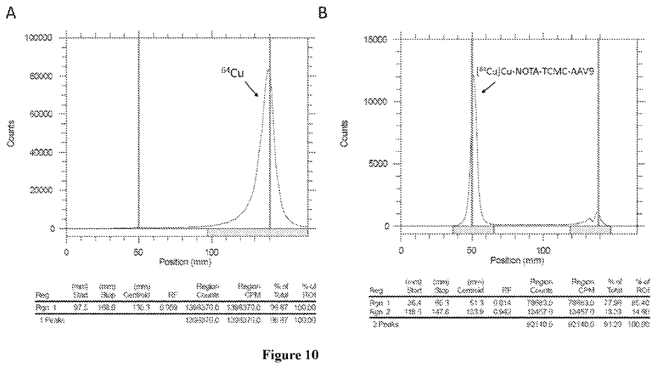

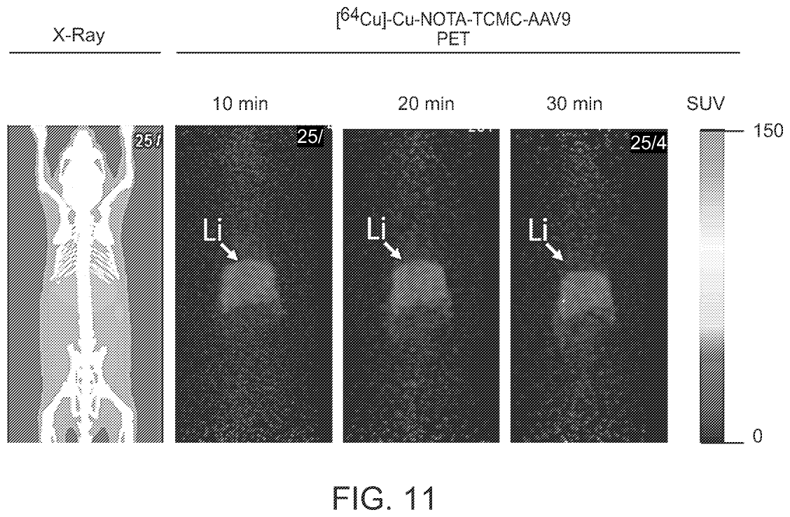

The radiosynthesis of [⁶⁴Cu]Cu-NOTA-TCMC-Adeno-Associated Viruses ([⁶⁴Cu]Cu-NOTA-TCMC-AAVs) was performed using AAV9 (FIG. 9). For the radiosynthesis, AAV9 was first conjugated with SCN-TCMC-NOTA by incubating ~5.32×10¹¹ AAV9 virus particle (~100~L) with ~50 μg SCN-TCMC-NOTA (5 μL of 10 mg/mL SCN-TCMC-NOTA) in phosphate buffered saline at pH 9.0 (pH adjusted using 0.25 M Na₂CO₃) for ~16 h at 37° C. in a thermomixer shaking at 600 rpm. Following incubation, the NOTA conjugated AAV9 particles were separated from unreacted SCN-TCMC-NOTA using Sephadex G50 size exclusion column. The purified NOTA-TCMC-AAV9 fraction after size exclusion purification was identified by assessing presence of AAV9 using quantitative polymerase chain reaction (qPCR) technique. The fraction with highest number of AAV particles was used for chelation of ⁶⁴Cu. The chelation of ⁶⁴Cu (~0.37 mCi) with NOTA-TCMC-AAV9 were performed in phosphate buffered saline at pH 5.0 (pH adjusted using 10% phosphoric acid) for ~16 h at room temperature. The chelation efficiency of ~85.4% was achieved as assessed by radio-TLC with mobile phase, 100 mM sodium citrate pH 5.0. In the radio-TLC chromatogram, the origin (Rf=~0) represented intact [⁶⁴Cu]Cu-NOTA-TCMC-AAV9 and solvent front (Rf=~1) represented unchelated ⁶⁴Cu (FIG. 10). After chelation, the [⁶⁴Cu]Cu-NOTA-TCMC-AAV9 were separated from free ⁶⁴Cu using Sephadex G50 size exclusion column. The purified [⁶⁴Cu]Cu-NOTA-TCMC-AAV9 (~86.2% radiochemical purity) was intravenously administered via tail vein injection in BALB/c mouse and PET images were acquired at different time points post-injection (FIG. 11). The [⁶⁴Cu]Cu-NOTA-TCMC-AAV9 primarily distributed into liver post-injection.

### Example 2

### Radiosynthesis and Preclinical Evaluation of [⁶⁴Cu]Cu-NOTA-TCMC-EVs

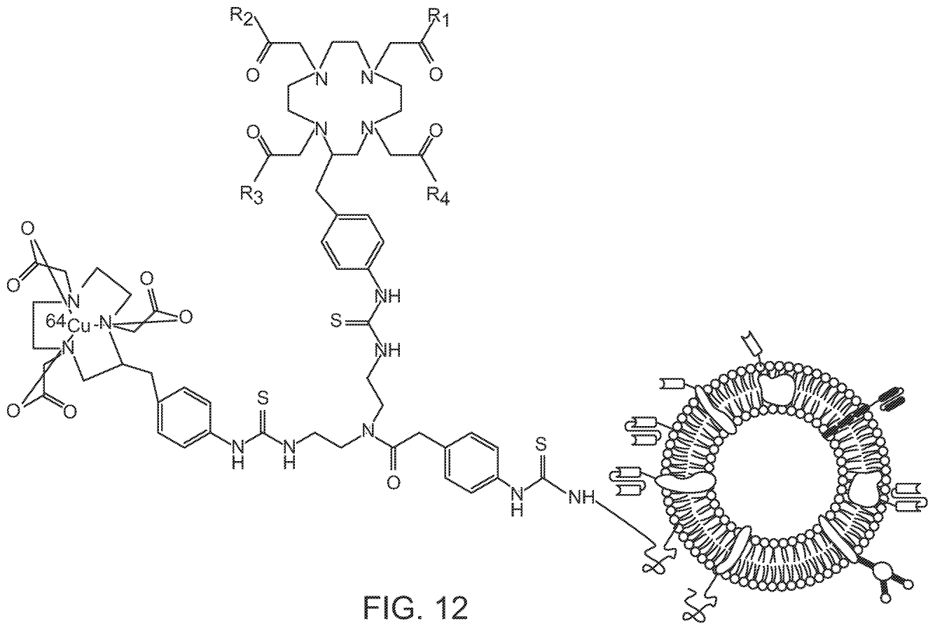

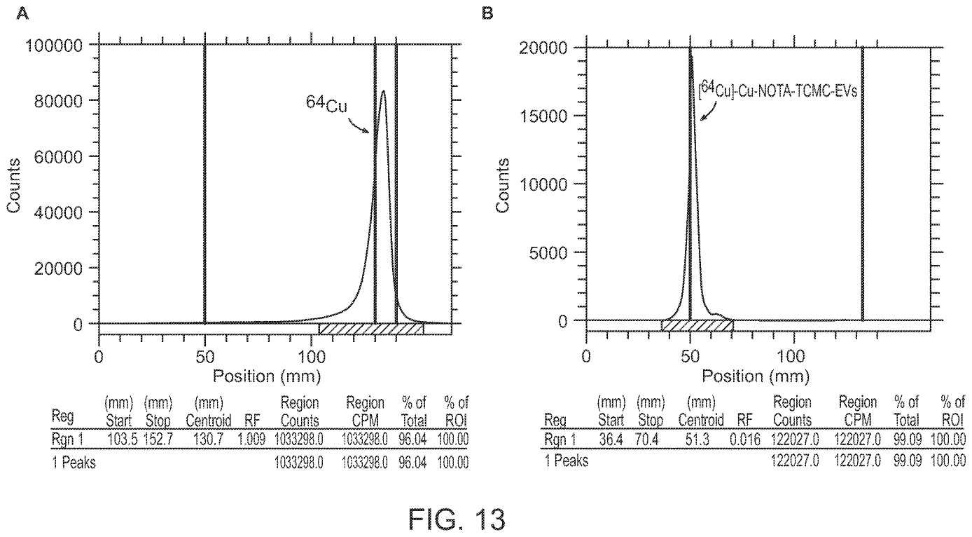

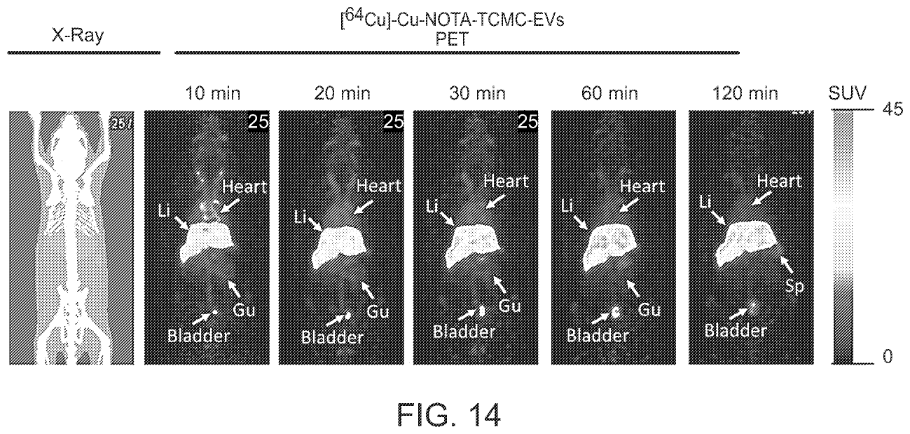

FIG. 12 contains a chemical structure of [⁶⁴Cu]Cu-NOTA-TCMC-Extracellular Vesicle ([⁶⁴Cu]Cu-NOTA-TCMC-EV). The radiosynthesis of [⁶⁴Cu]Cu-NOTA-TCMC-extracellular vesicles ([⁶⁴Cu]Cu-NOTA-TCMC-EVs) was performed using EVs derived from plasma (FIG. 12). For the radiosynthesis, plasma derived EVs was first conjugated with SCN-TCMC-NOTA by incubating ~100 g EVs (~100 L) with ~100 μg SCN-TCMC-NOTA (10 μL of 1 mg SCN-TCMC-NOTA/0.1 mL H₂O) in phosphate buffered saline at pH 9.0 (pH adjusted using 0.25 M Na₂CO₃) for 60 min at 37° C. in a thermomixer shaking at 600 rpm. Following incubation, the NOTA conjugated EVs were separated from unreacted SCN-TCMC-NOTA using Sephadex G50 resin size exclusion column. The purified NOTA-TCMC-EVs fraction after size exclusion purification was identified by assessing protein concentration in different fractions by Bradford assay. The fraction with highest protein concentration were used for chelation of ⁶⁴Cu. The chelation of ⁶⁴Cu (~1.45 mCi) with NOTA-TCMC-EVs were performed in phosphate buffered saline at pH 5.0 (pH adjusted using 10% phosphoric acid) for 10 min at room temperature. The chelation efficiency of ~100% was achieved as assessed by radio-TLC with mobile phase, 100 mM sodium citrate pH 5.0. In the radio-TLC chromatogram, the origin (Rf=~0) represented intact [⁶⁴Cu]Cu-NOTA-TCMC-EVs and solvent front (Rf=~1) represented unchelated ⁶⁴Cu (FIG. 13). After chelation, the [⁶⁴Cu]Cu-NOTA-TCMC-EVs were separated from free ⁶⁴Cu using Sephadex G50 resin size exclusion column. The purified [⁶⁴Cu]Cu-NOTA-TCMC-EVs (~100% radiochemical purity) was intravenously administered via tail vein injection in BALB/c mouse and PET images were acquired at different time points post-injection (FIG. 14). The [⁶⁴Cu]Cu-NOTA-TCMC-EVs primarily distributed into liver, heart, gut and bladder post-injection.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A conjugate comprising two or more chelators and a binding moiety, wherein one of said chelators is a chelator of an imaging isotope and one of said chelators is a chelator of a radiotherapy isotope,
wherein at least one chelator within the conjugate comprises one or more functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, —C(═S)NH₂, and C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
provided that the functional groups in said at least one chelator are not all C(═O)NH₂ or C(═O)OH;
and wherein said chelators and said binding moiety are linked via a moiety of Formula (I): 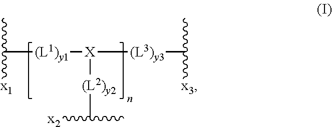
wherein:
each X is independently selected from N, P, P(═O), CR^{N}, and a moiety of formula (i): 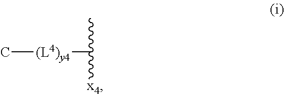
each of x₁, x₂, x₃, and x₄ independently indicates a point of attachment of the moiety of Formula (I) to said chelators or said binding moiety;
each of L₁, L², L³, and L⁴ is independently selected from C(═O), C(═S), N(R^{N}), O, S, S(═O), S(═O)₂, —CR^{N}=NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
each of y₁, y₂, y₃, and y₄ is independently an integer from 1 to 10;
each R^{N} is independently selected from H, C₁₋₃ alkyl, and C₁₋₃ haloalkyl; and
n is an integer selected from 1, 2, 3, 4, and 5.

2. The conjugate of claim 1, wherein said compound of Formula (I) has formula:

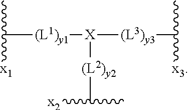

3. The conjugate of claim 2, wherein herein the moiety of Formula (I) has any one of the following formulae:

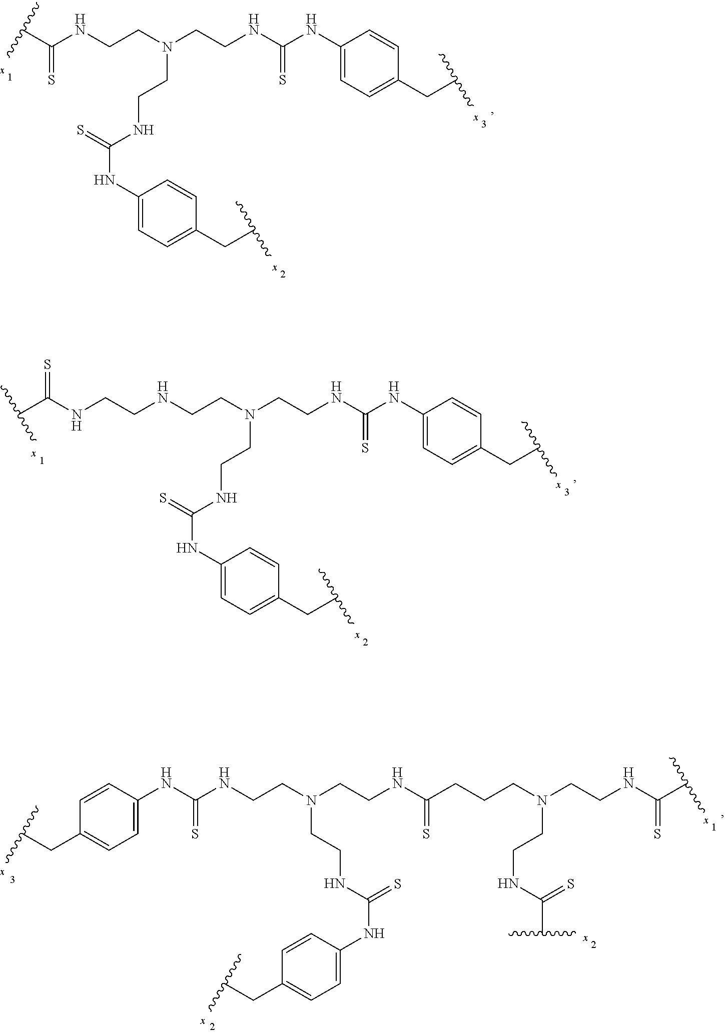 [SMILES: *Cc1ccc(NC(=S)NCCN(CCNC(*)=S)CCNC(=S)Nc2ccc(C*)cc2)cc1 ; *Cc1ccc(NC(=S)NCCN(CCNCCNC(*)=S)CCNC(=S)Nc2ccc(C*)cc2)cc1 ; *Cc1ccc(NC(=S)NCCN(CCNC(=S)CCCN(CCNC(*)=S)CCNC(*)=S)CCNC(=S)Nc2ccc(C*)cc2)cc1]

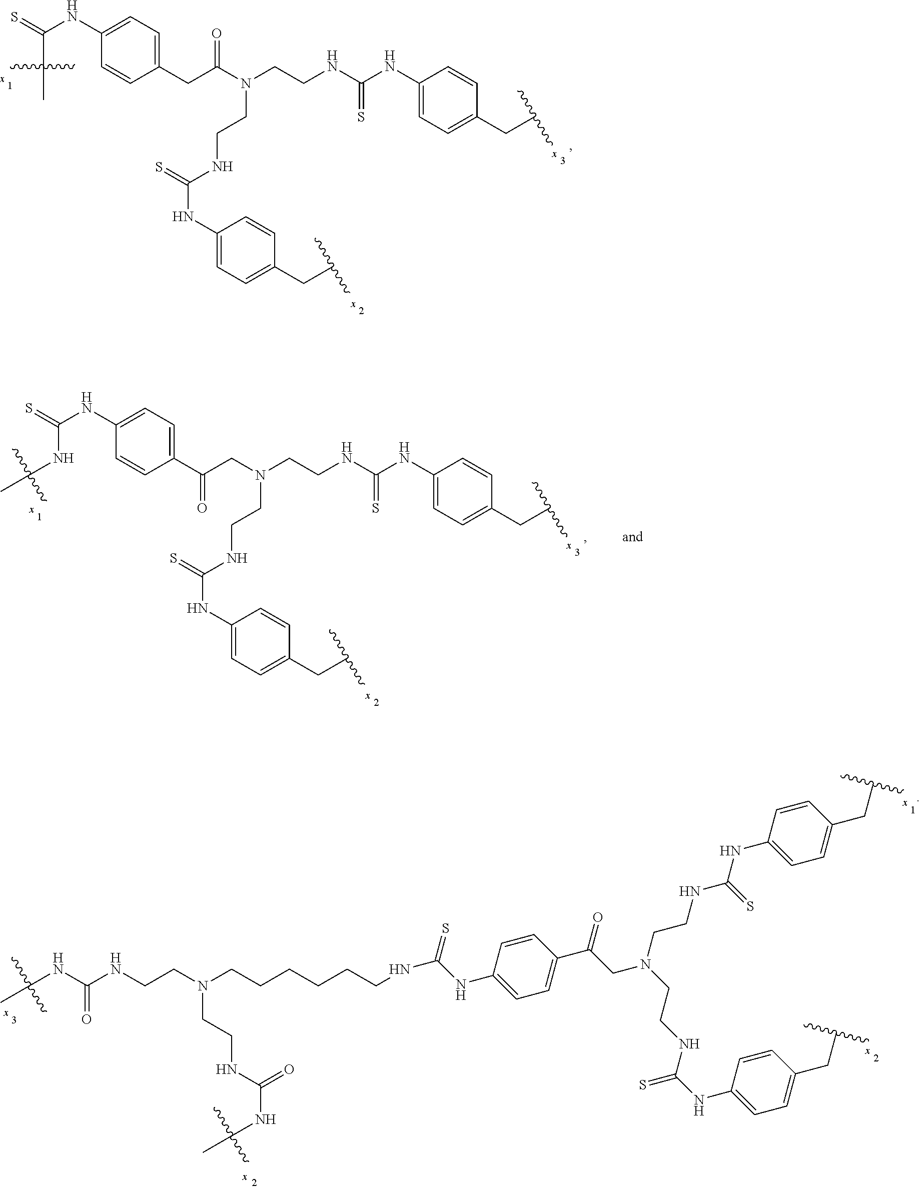 [SMILES: *Cc1ccc(NC(=S)NCCN(CCNC(=S)Nc2ccc(C*)cc2)C(=O)Cc2ccc(NC(*)=S)cc2)cc1 ; *Cc1ccc(NC(=S)NCCN(CCNC(=S)Nc2ccc(C*)cc2)CC(=O)c2ccc(NC(=S)N*)cc2)cc1 ; *Cc1ccc(NC(=S)NCCN(CCNC(=S)Nc2ccc(C*)cc2)CC(=O)c2ccc(NC(=S)NCCCCCCN(CCNC(=O)N*)CCNC(=O)N*)cc2)cc1]

4. The conjugate of claim 1, wherein said chelator of an imaging isotope independently comprises a compound selected from the group consisting of 1,4,7-triazacyclononane-1,4,7-triacetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA), each of which comprises one or more functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, —C(═S)NH₂, and C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

5. The conjugate of claim 4, wherein said chelator of an imaging isotope is substituted with 3 or 4 functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, provided that at least one functional group is other than C(═O)NH₂ or C(═O)OH.

6. The conjugate of claim 4, wherein said chelator of an imaging isotope has formula: 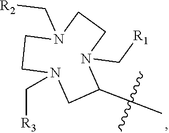
wherein each R¹, R², and R³ is independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and [symbol]
indicates a point of attachment to Formula (I).

7. The conjugate of claim 6, wherein at least one of R¹, R², and R³ is other than C(═O)NH₂ or C(═O)OH.

8-9. (canceled)

10. The conjugate of claim 1, wherein said chelator of a radiotherapy isotope independently comprises a compound selected from the group consisting of 1,4,7-triazacyclononane-1,4,7-triacetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA), each of which comprises one or more functional groups independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, —C(═S)NH₂, and C(═O)OR^{a}, wherein R^{a} is selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

11. (canceled)

12. The conjugate of claim 10, wherein said chelator of a radiotherapy isotope has formula: 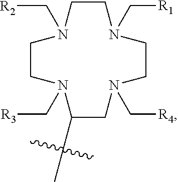
wherein each R¹, R², R³, and R⁴ is independently selected from C(═O)NH₂, C(═O)OH, —S(═O)₂OH, —S(═O)₂NH₂, —OP(═O)(OH)₂, —P(═O)(OH)₂, —OP(═O)(OH)NH₂, —C(═O)NHOH, —C(═S)SH, —C(═O)SH, —C(═S)OH, and —C(═S)NH₂, and [symbol]
indicates a point of attachment to Formula (I).

13-22. (canceled)

23. A method of treating cancer in a mammal in need thereof, wherein said method comprises administering a conjugate of claim 1 to said mammal, wherein said conjugate comprises said imaging isotope complexed to said chelator of said imaging isotope and wherein said conjugate comprises said radiotherapy isotope complexed to said chelator of said radiotherapy isotope.

24-28. (canceled)

29. A conjugate comprising two or more chelators and a binding moiety, wherein one of said chelators is a chelator of an imaging isotope and one of said chelators is a chelator of a radiotherapy isotope,
wherein the binding moiety is selected from an extracellular vesicle, a virus, a nucleic acid, and a glucose, or a derivative thereof,
and wherein said chelators and said binding moiety are linked via a moiety of Formula (I): 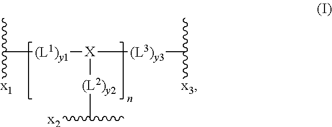
wherein:
each X is independently selected from N, P, P(═O), CR^{N}, and a moiety of formula (i): 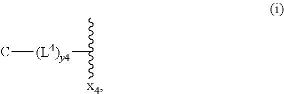
each of x₁, x₂, x₃, and x₄ independently indicates a point of attachment of the moiety of Formula (I) to said chelators or said binding moiety;
each of L¹, L², L³, and L⁴ is independently selected from C(═O), C(═S), N(R^{N}), O, S, S(═O), S(═O)₂, —CR^{N}=NR^{N}—, (—C₁₋₃ alkylene-O—)ₓ, (—O—C₁₋₃ alkylene-)ₓ, —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene, wherein each x is independently an integer from 1 to 10 and each of said —C₁₋₃ alkylene-, C₂₋₆ alkenylene, C₂₋₆ alkynylene, C₃₋₁₀ cycloalkylene, C₆₋₁₀ arylene, 5-14 membered heteroarylene, and 4-10 membered heterocycloalkylene is optionally substituted with 1, 2, or 3 substituents independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, carboxy, and C₁₋₃ alkoxycarbonyl;
each of y₁, y₂, y₃, and y₄ is independently an integer from 1 to 10;
each R^{N} is independently selected from H, C₁₋₃ alkyl, and C₁₋₃ haloalkyl; and
n is an integer selected from 1, 2, 3, 4, and 5.

30. The conjugate or claim 29, wherein the binding moiety is an extracellular vesicle.

31. The conjugate or claim 29, wherein the binding moiety is a virus.

32. The conjugate or claim 29, wherein the binding moiety is a nucleic acid.

33. The conjugate of claim 29, wherein said compound of Formula (I) has formula:

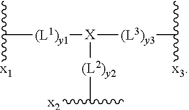

34. The conjugate of claim 33, wherein herein the moiety of Formula (I) has any one of the following formulae:

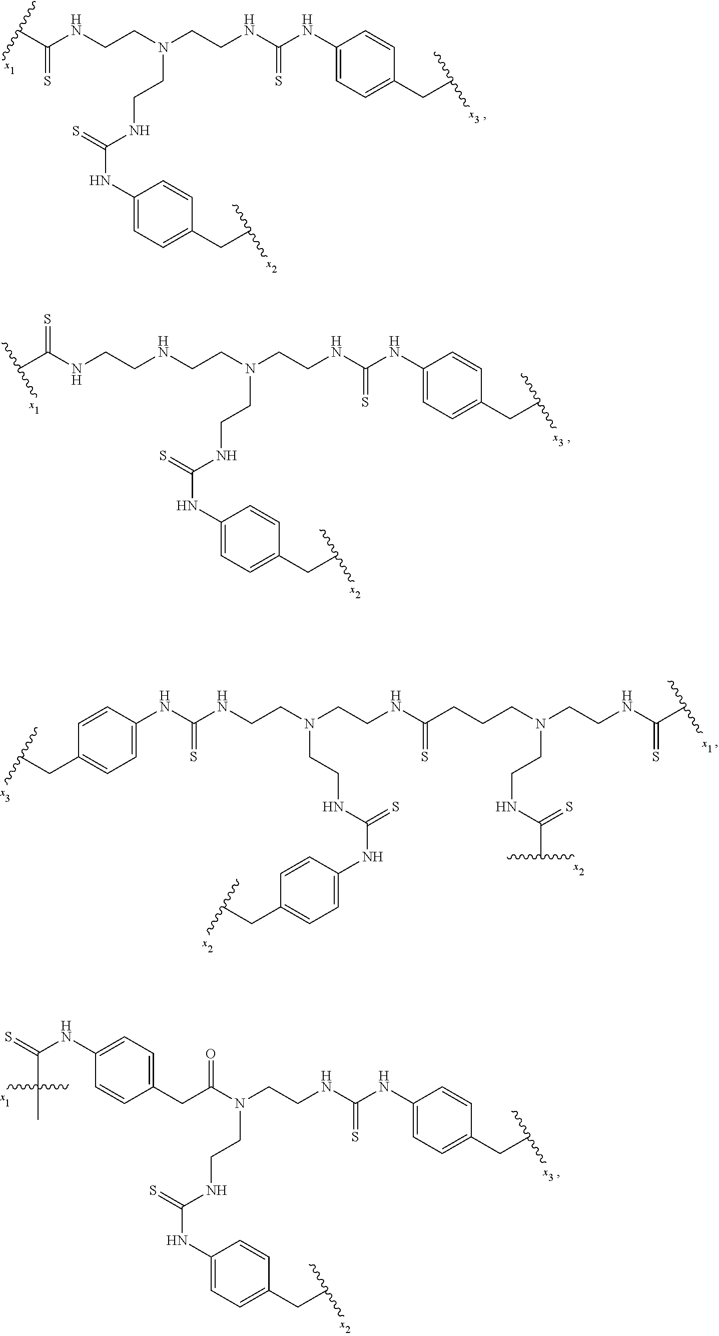

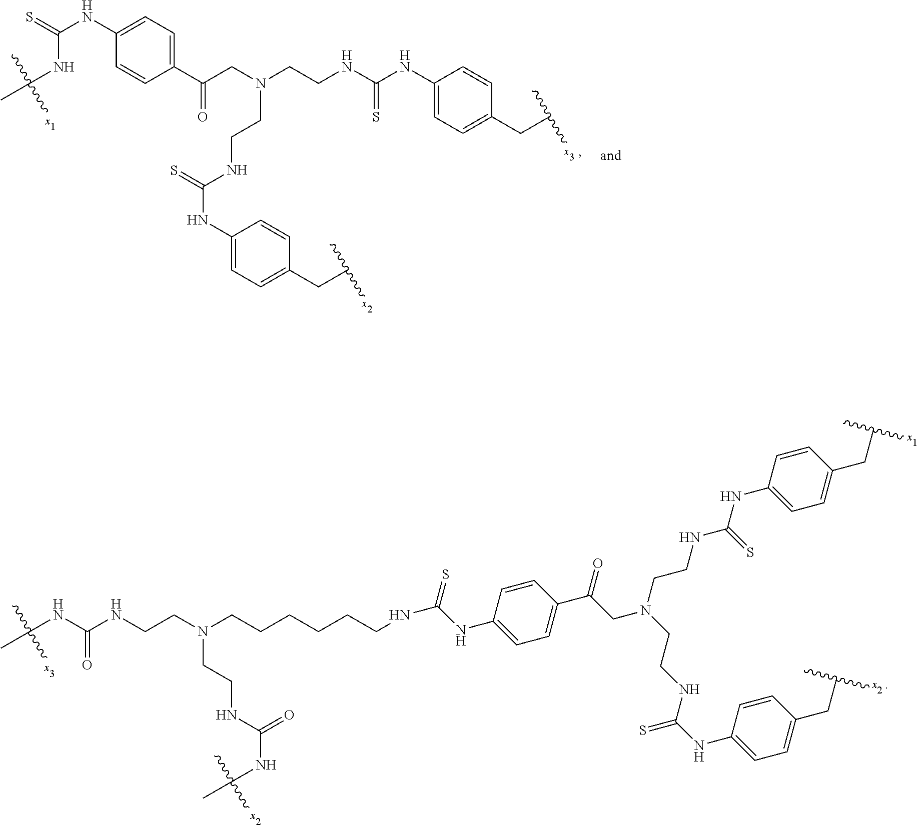

35. The conjugate of claim 29, wherein said chelator of an imaging isotope independently comprises a compound selected from the group consisting of 1,4,7-triazacyclononane-1,4,7-triacetic acid (NOTA), 2,2′,2″,2′″-(1,4,7,10-Tetraazacyclododecane-1,4,7,10-tetrayl)tetraacetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA).

36. The conjugate of claim 29, wherein said chelator of a radiotherapy isotope independently comprises a compound selected from the group consisting of 1,4,7-triazacyclononane-1,4,7-triacetic acid (NOTA), dodecane tetracetic acid (DOTA), 1,4,7,10-tetrakis(carbamoylmethyl)-1,4,7,10-tetracyclododecane (TCMC), 1-N-(4-aminobenzyl)-3,6,10,13,16,19-hexazabicyclo[6.6.6]eicosane-1,8-diamine (DiAmSar), N,N-bis(2-hydroxybenzyl)ethylenediamine-N,N-diacetic acid (HBED), deferoxamine (DFO), diethylenetraminepentacetic acid (DTPA), and N,N′-bis[(6-carboxy-2-pyridil)methyl]-4,13-diaza-18crown-6 (MACROPA).

37-42. (canceled)

43. The conjugate of claim 29, selected from any one of the following compounds:

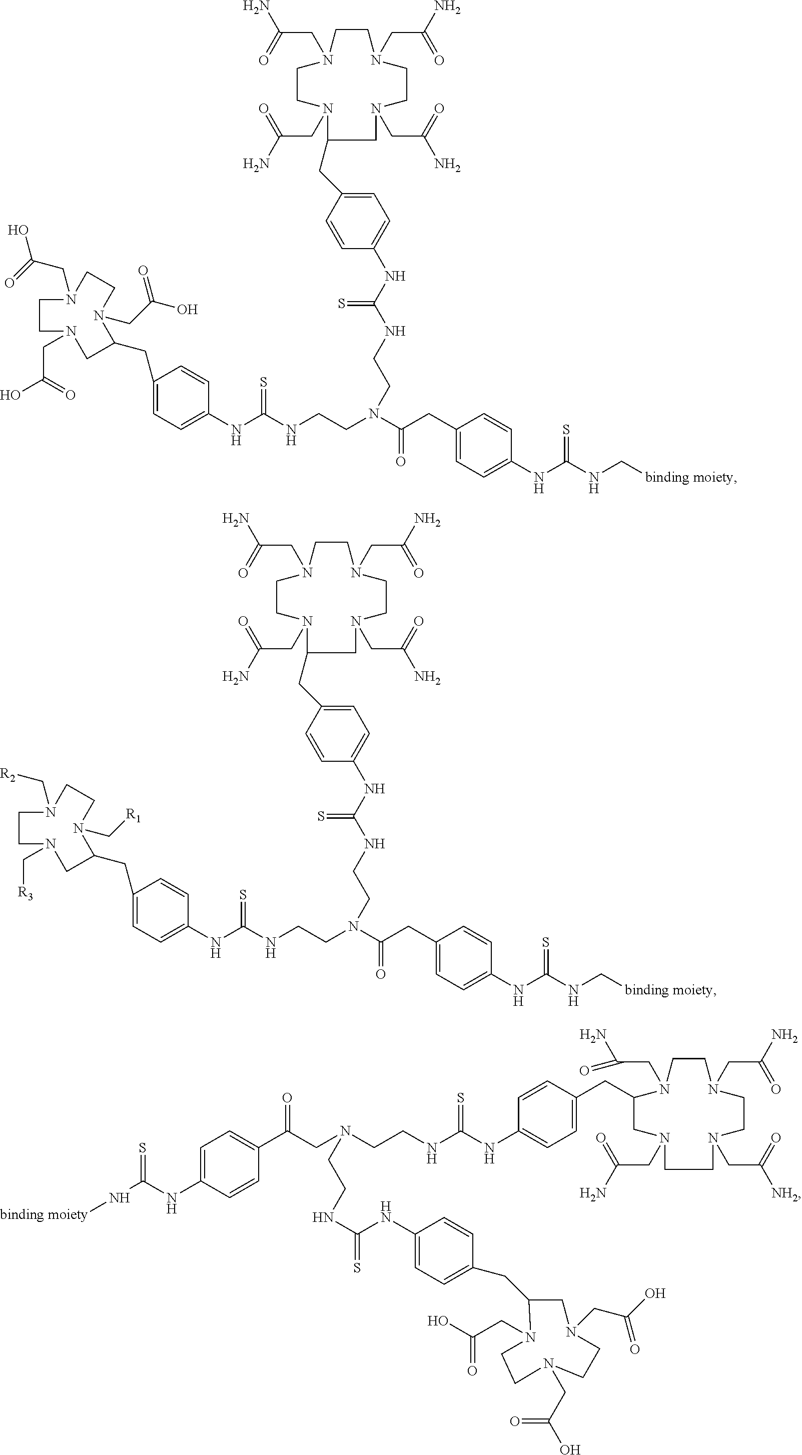

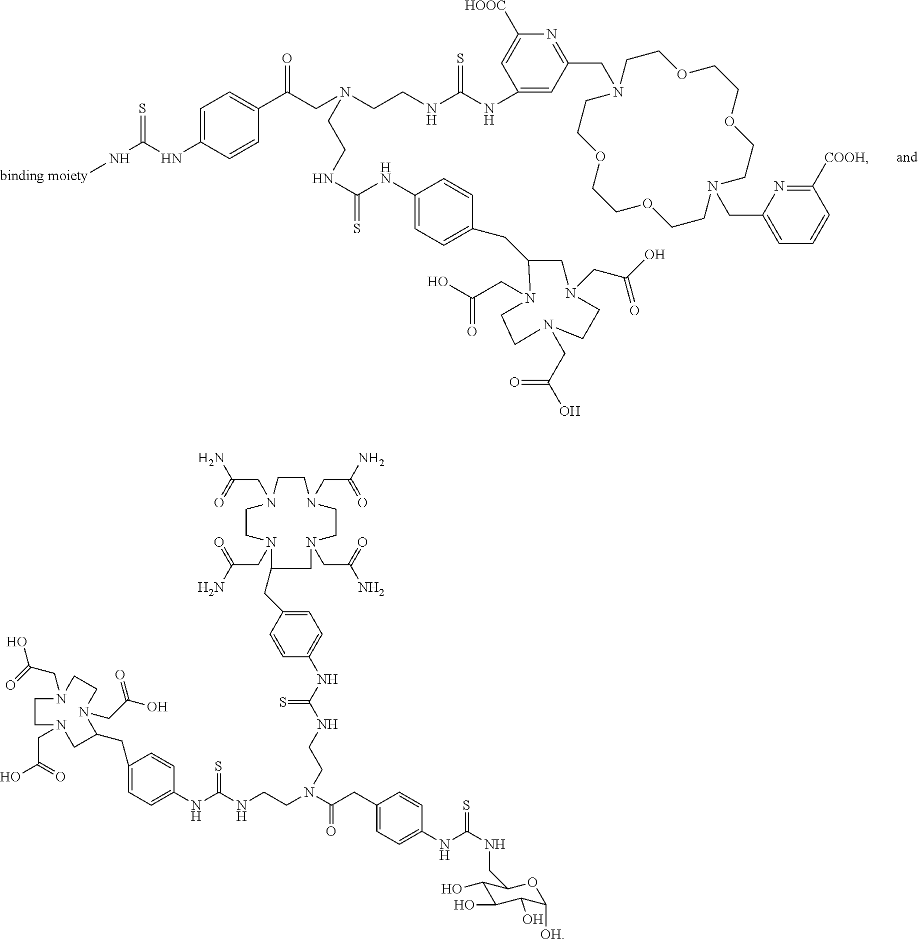

44. A method of treating cancer in a mammal in need thereof, wherein said method comprises administering a conjugate of claim 29 to said mammal, wherein said conjugate comprises said imaging isotope complexed to said chelator of said imaging isotope and wherein said conjugate comprises said radiotherapy isotope complexed to said chelator of said radiotherapy isotope.

45-60. (canceled)


SEQUENCE LISTING (712 sequences)

SEQ ID NO: 1 - protein, 6 aa, synthetic construct
QSINNY

SEQ ID NO: 2 - skipped

SEQ ID NO: 3 - protein, 10 aa, synthetic construct
QQSFSTPPIT

SEQ ID NO: 4 - protein, 8 aa, synthetic construct
GFTFADFT

SEQ ID NO: 5 - protein, 8 aa, synthetic construct
ISWNSNSI

SEQ ID NO: 6 - protein, 10 aa, synthetic construct
VKDRSGYSRF

SEQ ID NO: 7 - protein, 16 aa, synthetic construct
RSSQSLVHSNGNTYLH

SEQ ID NO: 8 - protein, 7 aa, synthetic construct
KVSNRFS

SEQ ID NO: 9 - protein, 9 aa, synthetic construct
SQSTHVPFT

SEQ ID NO: 10 - protein, 5 aa, synthetic construct
DYHLN

SEQ ID NO: 11 - protein, 18 aa, synthetic construct
IRNKRYGYRTEYSASVKG

SEQ ID NO: 12 - protein, 8 aa, synthetic construct
DFYDPFAY

SEQ ID NO: 13 - protein, 11 aa, synthetic construct
RASQSISSYLN

SEQ ID NO: 14 - protein, 7 aa, synthetic construct
AASSLQS

SEQ ID NO: 15 - protein, 8 aa, synthetic construct
QQSYKPYT

SEQ ID NO: 16 - protein, 5 aa, synthetic construct
SYAMS

SEQ ID NO: 17 - protein, 17 aa, synthetic construct
AISGSGGTTYYADSVKG

SEQ ID NO: 18 - protein, 8 aa, synthetic construct
QQSYKPYT

SEQ ID NO: 19 - protein, 11 aa, synthetic construct
RASQSVSSYLA

SEQ ID NO: 20 - protein, 7 aa, synthetic construct
DASNRAT

SEQ ID NO: 21 - protein, 10 aa, synthetic construct
QQRSNWPPLT

SEQ ID NO: 22 - protein, 5 aa, synthetic construct
GYGMH

SEQ ID NO: 23 - protein, 17 aa, synthetic construct
VIWYDGSKKYYVDSVKG

SEQ ID NO: 24 - protein, 9 aa, synthetic construct
QMGYWHFDL

SEQ ID NO: 25 - protein, 10 aa, synthetic construct
RASSSVSYIH

SEQ ID NO: 26 - protein, 7 aa, synthetic construct
ATSNLAS

SEQ ID NO: 27 - protein, 9 aa, synthetic construct
QQWTSNPPT

SEQ ID NO: 28 - protein, 5 aa, synthetic construct
SYNMH

SEQ ID NO: 29 - protein, 17 aa, synthetic construct
AIYPGNGDTSYNQKFKG

SEQ ID NO: 30 - protein, 12 aa, synthetic construct
STYYGGDWYFDV

SEQ ID NO: 31 - protein, 12 aa, synthetic construct
QSLYNSRTRKNY

SEQ ID NO: 32 - skipped

SEQ ID NO: 33 - protein, 8 aa, synthetic construct
KQSYNLRT

SEQ ID NO: 34 - protein, 4 aa, synthetic construct
TDYY

SEQ ID NO: 35 - protein, 10 aa, synthetic construct
IRNKANGYTT

SEQ ID NO: 36 - protein, 7 aa, synthetic construct
DIPGFAY

SEQ ID NO: 37 - protein, 11 aa, synthetic construct
KASQDVSIGVA

SEQ ID NO: 38 - protein, 7 aa, synthetic construct
SASYRYS

SEQ ID NO: 39 - protein, 9 aa, synthetic construct
QQYYIYPYT

SEQ ID NO: 40 - protein, 10 aa, synthetic construct
GFTFTDYTMD

SEQ ID NO: 41 - protein, 17 aa, synthetic construct
DVNPNSGGSIYNQRFKG

SEQ ID NO: 42 - protein, 10 aa, synthetic construct
NLGPSFYFDY

SEQ ID NO: 43 - protein, 11 aa, synthetic construct
SASQDISNYLN

SEQ ID NO: 44 - protein, 7 aa, synthetic construct
FTSSLHS

SEQ ID NO: 45 - protein, 9 aa, synthetic construct
QQYSTVPWT

SEQ ID NO: 46 - protein, 10 aa, synthetic construct
GYTFTNYGMN

SEQ ID NO: 47 - protein, 17 aa, synthetic construct
WLNTYTGEPTYAADFKR

SEQ ID NO: 48 - protein, 17 aa, synthetic construct
WLNTYTGEPTYAADFKR

SEQ ID NO: 49 - protein, 6 aa, synthetic construct
QGIRND

SEQ ID NO: 50 - protein, 12 aa, synthetic construct
SASSSISSNYLH

SEQ ID NO: 51 - protein, 11 aa, synthetic construct
RASQGISSALA

SEQ ID NO: 52 - protein, 11 aa, synthetic construct
RASQDISSALA

SEQ ID NO: 53 - protein, 11 aa, synthetic construct
RASQSVSSYLA

SEQ ID NO: 54 - protein, 11 aa, synthetic construct
KASQDVGTAVD

SEQ ID NO: 55 - protein, 11 aa, synthetic construct
KASENVGTYVS

SEQ ID NO: 56 - protein, 15 aa, synthetic construct
RASESIDSYDNTFMH

SEQ ID NO: 57 - protein, 11 aa, synthetic construct
KASQNVGSDVA

SEQ ID NO: 58 - protein, 6 aa, synthetic construct
KSISKY

SEQ ID NO: 59 - skipped

SEQ ID NO: 60 - protein, 7 aa, synthetic construct
RTSNLAS

SEQ ID NO: 61 - protein, 7 aa, synthetic construct
DASSLES

SEQ ID NO: 62 - protein, 7 aa, synthetic construct
DASNRAT

SEQ ID NO: 63 - protein, 7 aa, synthetic construct
WASTRHT

SEQ ID NO: 64 - protein, 7 aa, synthetic construct
GASNRFT

SEQ ID NO: 65 - protein, 7 aa, synthetic construct
RASILES

SEQ ID NO: 66 - protein, 7 aa, synthetic construct
STSYRYS

SEQ ID NO: 67 - skipped

SEQ ID NO: 68 - protein, 9 aa, synthetic construct
LQHNSHPYT

SEQ ID NO: 69 - protein, 9 aa, synthetic construct
QQGSYIPFT

SEQ ID NO: 70 - protein, 8 aa, synthetic construct
QQNSYPLT

SEQ ID NO: 71 - protein, 9 aa, synthetic construct
QQFNSYPLT

SEQ ID NO: 72 - protein, 10 aa, synthetic construct
QQRSNWPLFT

SEQ ID NO: 73 - protein, 9 aa, synthetic construct
GQSYTFPYT

SEQ ID NO: 74 - protein, 9 aa, synthetic construct
HQSIEDPYT

SEQ ID NO: 75 - protein, 9 aa, synthetic construct
QQYNSYPLT

SEQ ID NO: 76 - protein, 9 aa, synthetic construct
QQHIEYPWT

SEQ ID NO: 77 - protein, 8 aa, synthetic construct
GFTFITYG

SEQ ID NO: 78 - protein, 8 aa, synthetic construct
GFTFSNYN

SEQ ID NO: 79 - protein, 8 aa, synthetic construct
GFSFSGYG

SEQ ID NO: 80 - protein, 8 aa, synthetic construct
GFTFSSYG

SEQ ID NO: 81 - protein, 10 aa, synthetic construct
GFTFSDFYMY

SEQ ID NO: 82 - protein, 5 aa, synthetic construct
SYAMH

SEQ ID NO: 83 - protein, 5 aa, synthetic construct
SNWIG

SEQ ID NO: 84 - protein, 5 aa, synthetic construct
SNWIG

SEQ ID NO: 85 - protein, 5 aa, synthetic construct
NYWIG

SEQ ID NO: 86 - protein, 10 aa, synthetic construct
GYTFTEYTIH

SEQ ID NO: 87 - protein, 10 aa, synthetic construct
GFTFSNYWMN

SEQ ID NO: 88 - protein, 10 aa, synthetic construct
GYTFGTYVMH

SEQ ID NO: 89 - protein, 10 aa, synthetic construct
GFSLTAYGIN

SEQ ID NO: 90 - protein, 11 aa, synthetic construct
SGYTFTDYYMH

SEQ ID NO: 91 - protein, 8 aa, synthetic construct
IYYDESNK

SEQ ID NO: 92 - protein, 8 aa, synthetic construct
ISTGSSDI

SEQ ID NO: 93 - protein, 8 aa, synthetic construct
MSYDGSNK

SEQ ID NO: 94 - protein, 8 aa, synthetic construct
TWYDGSNK

SEQ ID NO: 95 - protein, 8 aa, synthetic construct
ISYAGNNK

SEQ ID NO: 96 - protein, 17 aa, synthetic construct
TISDGGGYTSYPDSVKG

SEQ ID NO: 97 - protein, 16 aa, synthetic construct
VISYDQNNKYYADSVK

SEQ ID NO: 98 - protein, 16 aa, synthetic construct
IIYPQDSDTRYSPSFQ

SEQ ID NO: 99 - protein, 16 aa, synthetic construct
IIYPGDSDTRYSPSFQ

SEQ ID NO: 100 - protein, 17 aa, synthetic construct
IIYPGDSDTRYSPSFQQ

SEQ ID NO: 101 - protein, 17 aa, synthetic construct
NINPNNGGTTYNQKFED

SEQ ID NO: 102 - protein, 19 aa, synthetic construct
EIRSQSNNFATHYAESVKG

SEQ ID NO: 103 - protein, 17 aa, synthetic construct
YINPYNDVTRYNGKFKG

SEQ ID NO: 104 - protein, 16 aa, synthetic construct
VIWPCGNTDYNSTLKS

SEQ ID NO: 105 - protein, 10 aa, synthetic construct
YFNPYNDYTR

SEQ ID NO: 106 - protein, 6 aa, synthetic construct
YYGMDV

SEQ ID NO: 107 - protein, 10 aa, synthetic construct
ARAPRVAVEE

SEQ ID NO: 108 - protein, 10 aa, synthetic construct
DYSYYYGMDV

SEQ ID NO: 109 - protein, 10 aa, synthetic construct
ARDIIGTTRD

SEQ ID NO: 110 - protein, 17 aa, synthetic construct
AKDGYYDFLTFDYTLDY

SEQ ID NO: 111 - protein, 15 aa, synthetic construct
ARDGNWGGPYWYFDL

SEQ ID NO: 112 - protein, 17 aa, synthetic construct
AKDPYYDFLTGSDYFDY

SEQ ID NO: 113 - protein, 10 aa, synthetic construct
GLWLRDALDY

SEQ ID NO: 114 - protein, 15 aa, synthetic construct
AVPWQSRYYYYQMDV

SEQ ID NO: 115 - protein, 10 aa, synthetic construct
QTGFLWSSDL

SEQ ID NO: 116 - protein, 10 aa, synthetic construct
QTGFLWSFDL

SEQ ID NO: 117 - protein, 12 aa, synthetic construct
PGYTSSWTSFDY

SEQ ID NO: 118 - protein, 6 aa, synthetic construct
GWNFDY

SEQ ID NO: 119 - protein, 5 aa, synthetic construct
RWNNF

SEQ ID NO: 120 - protein, 9 aa, synthetic construct
GENWYYFDS

SEQ ID NO: 121 - protein, 13 aa, synthetic construct
DSYGNFKRGWFDF

SEQ ID NO: 122 - protein, 14 aa, synthetic construct
CARSDGYYDAMDYW

SEQ ID NO: 123 - protein, 17 aa, synthetic construct
KSSQSLLNSRNRKNYLA

SEQ ID NO: 124 - protein, 17 aa, synthetic construct
KSSQSLLNSRTRKNYLA

SEQ ID NO: 125 - protein, 7 aa, synthetic construct
WASTRES

SEQ ID NO: 126 - protein, 7 aa, synthetic construct
WASTRAS

SEQ ID NO: 127 - protein, 9 aa, synthetic construct
SQSTRVPFT

SEQ ID NO: 128 - protein, 8 aa, synthetic construct
KQSYYLWT

SEQ ID NO: 129 - protein, 5 aa, synthetic construct
DYHMN

SEQ ID NO: 130 - protein, 5 aa, synthetic construct
DYGMA

SEQ ID NO: 131 - protein, 19 aa, synthetic construct
LIRNKANGYRTEYSASVKG

SEQ ID NO: 132 - protein, 17 aa, synthetic construct
FISNLGYSIYYADSVKG

SEQ ID NO: 133 - protein, 17 aa, synthetic construct
FISNLAYSIYYADSVKG

SEQ ID NO: 134 - protein, 13 aa, synthetic construct
APYDYDSFYPMDY

SEQ ID NO: 135 - protein, 13 aa, synthetic construct
APYDYDSFDPMDY

SEQ ID NO: 136 - protein, 17 aa, synthetic construct
KSSQSLLYSRNQKNYLA

SEQ ID NO: 137 - protein, 12 aa, synthetic construct
RASQSVSRNYLA

SEQ ID NO: 138 - protein, 12 aa, synthetic construct
RASQSVTSSYLA

SEQ ID NO: 139 - protein, 7 aa, synthetic construct
WASTRES

SEQ ID NO: 140 - protein, 7 aa, synthetic construct
GASSRAT

SEQ ID NO: 141 - protein, 7 aa, synthetic construct
GASTRAT

SEQ ID NO: 142 - protein, 7 aa, synthetic construct
VGSRRAT

SEQ ID NO: 143 - protein, 9 aa, synthetic construct
QQSYSTPRT

SEQ ID NO: 144 - protein, 8 aa, synthetic construct
QQFSYPLT

SEQ ID NO: 145 - protein, 9 aa, synthetic construct
QQSLGYPPT

SEQ ID NO: 146 - protein, 9 aa, synthetic construct
QQGQVIPPT

SEQ ID NO: 147 - protein, 5 aa, synthetic construct
SYAMS

SEQ ID NO: 148 - protein, 5 aa, synthetic construct
SYAMS

SEQ ID NO: 149 - protein, 5 aa, synthetic construct
SYAMN

SEQ ID NO: 150 - protein, 5 aa, synthetic construct
SYAMS

SEQ ID NO: 151 - protein, 4 aa, synthetic construct
RYAM

SEQ ID NO: 152 - protein, 17 aa, synthetic construct
AISGSGGGTRYADSVKG

SEQ ID NO: 153 - protein, 16 aa, synthetic construct
GISGSGGTYYADSVKG

SEQ ID NO: 154 - protein, 16 aa, synthetic construct
TISSSGRTYYADSVKG

SEQ ID NO: 155 - protein, 17 aa, synthetic construct
GISGGGGSTYYADSVKG

SEQ ID NO: 156 - protein, 15 aa, synthetic construct
SISAGGTYYADSVKG

SEQ ID NO: 157 - protein, 11 aa, synthetic construct
HSSGFHWYFDY

SEQ ID NO: 158 - protein, 12 aa, synthetic construct
ISFYPGGTYFDY

SEQ ID NO: 159 - protein, 9 aa, synthetic construct
GLVASAPFD

SEQ ID NO: 160 - protein, 12 aa, synthetic construct
IAHSRIGWHFDY

SEQ ID NO: 161 - protein, 12 aa, synthetic construct
TFSGYAHYDFDY

SEQ ID NO: 162 - protein, 11 aa, synthetic construct
WASQGISSYLA

SEQ ID NO: 163 - protein, 11 aa, synthetic construct
RASQGISSALA

SEQ ID NO: 164 - protein, 7 aa, synthetic construct
GASSRAT

SEQ ID NO: 165 - protein, 7 aa, synthetic construct
YASSLQS

SEQ ID NO: 166 - protein, 7 aa, synthetic construct
DASSLGS

SEQ ID NO: 167 - protein, 7 aa, synthetic construct
DASSLES

SEQ ID NO: 168 - protein, 9 aa, synthetic construct
QQYGSSPIT

SEQ ID NO: 169 - protein, 8 aa, synthetic construct
QQYYSTLT

SEQ ID NO: 170 - protein, 9 aa, synthetic construct
QQRSNWPWT

SEQ ID NO: 171 - protein, 9 aa, synthetic construct
QQFNSYPIT

SEQ ID NO: 172 - protein, 5 aa, synthetic construct
SYGMH

SEQ ID NO: 173 - protein, 17 aa, synthetic construct
IIWYDGSKKNYADSVKG

SEQ ID NO: 174 - protein, 17 aa, synthetic construct
AIWYNGRKQDYADSVKG

SEQ ID NO: 175 - protein, 9 aa, synthetic construct
GTGYNWFDP

SEQ ID NO: 176 - protein, 10 aa, synthetic construct
RASSSLSFMH

SEQ ID NO: 177 - protein, 10 aa, synthetic construct
RASSSVSYMH

SEQ ID NO: 178 - protein, 15 aa, synthetic construct
RASGSVDSFGNSFMH

SEQ ID NO: 179 - protein, 15 aa, synthetic construct
RASESVDNFGNSFMH

SEQ ID NO: 180 - protein, 7 aa, synthetic construct
RASNLES

SEQ ID NO: 181 - protein, 9 aa, synthetic construct
HQWSSNPLT

SEQ ID NO: 182 - protein, 9 aa, synthetic construct
QQSFSNPPT

SEQ ID NO: 183 - protein, 9 aa, synthetic construct
QQSYEDPFT

SEQ ID NO: 184 - protein, 5 aa, synthetic construct
NYGMN

SEQ ID NO: 185 - protein, 5 aa, synthetic construct
NVGMN

SEQ ID NO: 186 - protein, 10 aa, synthetic construct
WINTYTGEPS

SEQ ID NO: 187 - protein, 10 aa, synthetic construct
WINTYTGEPA

SEQ ID NO: 188 - protein, 16 aa, synthetic construct
AIYPGNGDTSYNQKFK

SEQ ID NO: 189 - protein, 13 aa, synthetic construct
SHYGSNYVDYFDV

SEQ ID NO: 190 - protein, 12 aa, synthetic construct
WYYSNSYWYFDV

SEQ ID NO: 191 - protein, 12 aa, synthetic construct
GAYYRYDLGMDY

SEQ ID NO: 192 - protein, 12 aa, synthetic construct
STYYGGDWYFNV

SEQ ID NO: 193 - protein, 11 aa, synthetic construct
QSFNSRTRKNY

SEQ ID NO: 194 - protein, 16 aa, synthetic construct
KSSQSLFNSRTRKNSL

SEQ ID NO: 195 - protein, 14 aa, synthetic construct
TGTISDVGGHNFVS

SEQ ID NO: 196 - protein, 13 aa, synthetic construct
TGNSNNVGNQGAA

SEQ ID NO: 197 - protein, 11 aa, synthetic construct
QGDSLRKFFAS

SEQ ID NO: 198 - protein, 11 aa, synthetic construct
RASQSVNTNLA

SEQ ID NO: 199 - protein, 11 aa, synthetic construct
QGDSLRSYYAS

SEQ ID NO: 200 - protein, 13 aa, synthetic construct
SGSRSNIGSNTVN

SEQ ID NO: 201 - protein, 13 aa, synthetic construct
SGSRSNIGGNTVN

SEQ ID NO: 202 - protein, 7 aa, synthetic construct
WASARDS

SEQ ID NO: 203 - protein, 7 aa, synthetic construct
WASIRES

SEQ ID NO: 204 - protein, 7 aa, synthetic construct
EVTKRPA

SEQ ID NO: 205 - protein, 7 aa, synthetic construct
RNNNRPS

SEQ ID NO: 206 - protein, 7 aa, synthetic construct
GKNSRPS

SEQ ID NO: 207 - protein, 7 aa, synthetic construct
GASSRAT

SEQ ID NO: 208 - protein, 7 aa, synthetic construct
GKNNRPS

SEQ ID NO: 209 - protein, 7 aa, synthetic construct
TNNQRPS

SEQ ID NO: 210 - protein, 7 aa, synthetic construct
ANNQRPS

SEQ ID NO: 211 - protein, 8 aa, synthetic construct
MQSFNLRT

SEQ ID NO: 212 - protein, 8 aa, synthetic construct
KQSTNLRT

SEQ ID NO: 213 - protein, 10 aa, synthetic construct
SSYGGSNDVI

SEQ ID NO: 214 - protein, 11 aa, synthetic construct
SAWDNRLKTYV

SEQ ID NO: 215 - protein, 11 aa, synthetic construct
NSRDSRDNHQV

SEQ ID NO: 216 - protein, 9 aa, synthetic construct
QHYGSSPLT

SEQ ID NO: 217 - protein, 9 aa, synthetic construct
NSRSGSQRV

SEQ ID NO: 218 - protein, 11 aa, synthetic construct
LSFDSSLTSYV

SEQ ID NO: 219 - protein, 11 aa, synthetic construct
AAWDDNLSGHW

SEQ ID NO: 220 - skipped

SEQ ID NO: 221 - protein, 8 aa, synthetic construct
GFTFTDNY

SEQ ID NO: 222 - protein, 8 aa, synthetic construct
GFSLTDYG

SEQ ID NO: 223 - protein, 5 aa, synthetic construct
DNTMS

SEQ ID NO: 224 - protein, 5 aa, synthetic construct
DTQVY

SEQ ID NO: 225 - protein, 5 aa, synthetic construct
SYGMH

SEQ ID NO: 226 - protein, 7 aa, synthetic construct
SNFVAWN

SEQ ID NO: 227 - protein, 5 aa, synthetic construct
SYGIS

SEQ ID NO: 228 - protein, 5 aa, synthetic construct
SYPMH

SEQ ID NO: 229 - protein, 5 aa, synthetic construct
NYGLH

SEQ ID NO: 230 - protein, 5 aa, synthetic construct
RYGMH

SEQ ID NO: 231 - protein, 5 aa, synthetic construct
SYAMS

SEQ ID NO: 232 - protein, 5 aa, synthetic construct
IHTMS

SEQ ID NO: 233 - protein, 5 aa, synthetic construct
DYAIG

SEQ ID NO: 234 - protein, 5 aa, synthetic construct
GYTIA

SEQ ID NO: 235 - protein, 5 aa, synthetic construct
PSAMA

SEQ ID NO: 236 - protein, 5 aa, synthetic construct
DYAMS

SEQ ID NO: 237 - skipped

SEQ ID NO: 238 - protein, 5 aa, synthetic construct
LNAMG

SEQ ID NO: 239 - protein, 5 aa, synthetic construct
VNDMG

SEQ ID NO: 240 - protein, 5 aa, synthetic construct
YYTIG

SEQ ID NO: 241 - protein, 5 aa, synthetic construct
YHAIV

SEQ ID NO: 242 - protein, 5 aa, synthetic construct
NYAMG

SEQ ID NO: 243 - protein, 5 aa, synthetic construct
INAMG

SEQ ID NO: 244 - protein, 7 aa, synthetic construct
IWGDGTT

SEQ ID NO: 245 - protein, 19 aa, synthetic construct
FIRNKANGYTTDYSASVRG

SEQ ID NO: 246 - protein, 16 aa, synthetic construct
MIWGDGTTDYNSALKS

SEQ ID NO: 247 - protein, 17 aa, synthetic construct
VISYDGSNKYYADSVKG

SEQ ID NO: 248 - protein, 18 aa, synthetic construct
RTYYRSRWYNDYAVSVQS

SEQ ID NO: 249 - protein, 17 aa, synthetic construct
WISAYNGNTNYAQKLQG

SEQ ID NO: 250 - protein, 17 aa, synthetic construct
VISSDGRNKYYPDSVKG

SEQ ID NO: 251 - protein, 17 aa, synthetic construct
NIKQDGSEKYYVDSVKG

SEQ ID NO: 252 - protein, 17 aa, synthetic construct
VISHDGTKKYYADSVKG

SEQ ID NO: 253 - protein, 17 aa, synthetic construct
LISYDGSKTFYGESVKG

SEQ ID NO: 254 - protein, 17 aa, synthetic construct
GIKSSGDSTRYAGSVKG

SEQ ID NO: 255 - protein, 17 aa, synthetic construct
TIKPSGDTTNYANAVKG

SEQ ID NO: 256 - protein, 17 aa, synthetic construct
AISWNGGSTDYADSVKG

SEQ ID NO: 257 - protein, 17 aa, synthetic construct
CISGSDGSTTYADSVKG

SEQ ID NO: 258 - protein, 17 aa, synthetic construct
YHRWSDGANLYADSVKG

SEQ ID NO: 259 - protein, 17 aa, synthetic construct
STIWSRGDTYFADSVKG

SEQ ID NO: 260 - protein, 17 aa, synthetic construct
AISWNGGSADYADSVKG

SEQ ID NO: 261 - protein, 17 aa, synthetic construct
TSRLITDNIIYADSVKG

SEQ ID NO: 262 - protein, 16 aa, synthetic construct
GITSSTSTYYADSVKG

SEQ ID NO: 263 - protein, 16 aa, synthetic construct
VITSGGGTNYVDSVKG

SEQ ID NO: 264 - protein, 17 aa, synthetic construct
CISSSDGSTAYLGSVQG

SEQ ID NO: 265 - protein, 17 aa, synthetic construct
CITSRDSITYYASFVKG

SEQ ID NO: 266 - protein, 21 aa, synthetic construct
AITRSGVRSGVSAIYGDSVKD

SEQ ID NO: 267 - protein, 16 aa, synthetic construct
SITSGGSTVYADSVKG

SEQ ID NO: 268 - protein, 11 aa, synthetic construct
ARDVGSNYFDY

SEQ ID NO: 269 - protein, 10 aa, synthetic construct
ARGRQFGFDY

SEQ ID NO: 270 - protein, 9 aa, synthetic construct
DVGSNYFDY

SEQ ID NO: 271 - protein, 8 aa, synthetic construct
GRQFGFDY

SEQ ID NO: 272 - protein, 12 aa, synthetic construct
DLVAAAGTAFDI

SEQ ID NO: 273 - protein, 8 aa, synthetic construct
GQHSGFDF

SEQ ID NO: 274 - protein, 17 aa, synthetic construct
DTPGIAARRYYYYGMDV

SEQ ID NO: 275 - protein, 12 aa, synthetic construct
GGYHDFWSGPDY

SEQ ID NO: 276 - protein, 18 aa, synthetic construct
DQVSGITIFGGKWRSPDV

SEQ ID NO: 277 - protein, 17 aa, synthetic construct
DGGYCSGGRCYSYGMDV

SEQ ID NO: 278 - protein, 13 aa, synthetic construct
ATVTTDGYYYMDV

SEQ ID NO: 279 - protein, 13 aa, synthetic construct
SRVSRTGLYTYDN

SEQ ID NO: 280 - protein, 7 aa, synthetic construct
DYFGTGV

SEQ ID NO: 281 - protein, 18 aa, synthetic construct
DQGPFYSGTYYYTRQYGY

SEQ ID NO: 282 - protein, 13 aa, synthetic construct
ARMTTSNDKEYLY

SEQ ID NO: 283 - protein, 18 aa, synthetic construct
QYGVGGRVVCPGPYEYDV

SEQ ID NO: 284 - protein, 13 aa, synthetic construct
AWMTTSNDKEYLY

SEQ ID NO: 285 - protein, 10 aa, synthetic construct
RVRPYGQYDY

SEQ ID NO: 286 - protein, 18 aa, synthetic construct
DQGPFYSGTYYYTKGYAY

SEQ ID NO: 287 - protein, 16 aa, synthetic construct
RQNYSRSVFGAKDYDY

SEQ ID NO: 288 - protein, 15 aa, synthetic construct
DCPDYYSDYECPLED

SEQ ID NO: 289 - protein, 11 aa, synthetic construct
YYSSGISTLRS

SEQ ID NO: 290 - protein, 20 aa, synthetic construct
BSADSRCSIGSIGFTWLYNN

SEQ ID NO: 291 - protein, 15 aa, synthetic construct
BTSMTCPTLIVRFNY

SEQ ID NO: 292 - protein, 15 aa, synthetic construct
SAIGSGALRRFEYDY

SEQ ID NO: 293 - protein, 15 aa, synthetic construct
DGVPEWGKVQYPDTY

SEQ ID NO: 294 - protein, 15 aa, synthetic construct
RASQSVSGSRFTYMH

SEQ ID NO: 295 - protein, 11 aa, synthetic construct
RASQDVNTAVA

SEQ ID NO: 296 - protein, 7 aa, synthetic construct
SASYRES

SEQ ID NO: 297 - protein, 7 aa, synthetic construct
SASYLYS

SEQ ID NO: 298 - protein, 7 aa, synthetic construct
SASYLES

SEQ ID NO: 299 - protein, 7 aa, synthetic construct
SASYRET

SEQ ID NO: 300 - protein, 7 aa, synthetic construct
SASYLYT

SEQ ID NO: 301 - protein, 7 aa, synthetic construct
SASYLET

SEQ ID NO: 302 - protein, 7 aa, synthetic construct
YASILES

SEQ ID NO: 303 - protein, 7 aa, synthetic construct
SASYRYT

SEQ ID NO: 304 - protein, 7 aa, synthetic construct
SASFLYS

SEQ ID NO: 305 - protein, 7 aa, synthetic construct
SASFRYT

SEQ ID NO: 306 - protein, 10 aa, synthetic construct
QHSWEIPPWT

SEQ ID NO: 307 - protein, 10 aa, synthetic construct
GFTFTDYTMS

SEQ ID NO: 308 - protein, 5 aa, synthetic construct
GYWMN

SEQ ID NO: 309 - protein, 10 aa, synthetic construct
GFNIKDTYIH

SEQ ID NO: 310 - protein, 10 aa, synthetic construct
GFTFNDYTMD

SEQ ID NO: 311 - protein, 16 aa, synthetic construct
MIHPLDAEIRANQKFR

SEQ ID NO: 312 - protein, 16 aa, synthetic construct
DVNPNSGGSIYNQRFK

SEQ ID NO: 313 - protein, 16 aa, synthetic construct
RIYPTNGYTRYADSVK

SEQ ID NO: 314 - protein, 16 aa, synthetic construct
DVNPNSGGSIVNRRFK

SEQ ID NO: 315 - protein, 9 aa, synthetic construct
GTYDGGFEY

SEQ ID NO: 316 - protein, 11 aa, synthetic construct
WGGDGFYAMDY

SEQ ID NO: 317 - protein, 10 aa, synthetic construct
NLGPFFYFDY

SEQ ID NO: 318 - protein, 11 aa, synthetic construct
RASQDVSTAVA

SEQ ID NO: 319 - protein, 11 aa, synthetic construct
RASQVIRRSLA

SEQ ID NO: 320 - protein, 11 aa, synthetic construct
RASQSYAYAVA

SEQ ID NO: 321 - protein, 12 aa, synthetic construct
CRASQASYYDVA

SEQ ID NO: 322 - protein, 7 aa, synthetic construct
SASFLYS

SEQ ID NO: 323 - protein, 7 aa, synthetic construct
AASNLAS

SEQ ID NO: 324 - protein, 7 aa, synthetic construct
DASYLYS

SEQ ID NO: 325 - protein, 7 aa, synthetic construct
AASYLYS

SEQ ID NO: 326 - protein, 9 aa, synthetic construct
QQSYTTPPT

SEQ ID NO: 327 - protein, 9 aa, synthetic construct
QQSNTSPLT

SEQ ID NO: 328 - protein, 9 aa, synthetic construct
QQAYSSPDT

SEQ ID NO: 329 - protein, 10 aa, synthetic construct
CQQYYYAPAT

SEQ ID NO: 330 - protein, 10 aa, synthetic construct
GYDFTNYGMN

SEQ ID NO: 331 - protein, 10 aa, synthetic construct
GYTFTHYGMN

SEQ ID NO: 332 - protein, 10 aa, synthetic construct
GYDFTHYGMN

SEQ ID NO: 333 - protein, 5 aa, synthetic construct
ASWIH

SEQ ID NO: 334 - protein, 5 aa, synthetic construct
GSWIF

SEQ ID NO: 335 - protein, 10 aa, synthetic construct
GFAISDYDIH

SEQ ID NO: 336 - protein, 5 aa, synthetic construct
DYDIH

SEQ ID NO: 337 - protein, 17 aa, synthetic construct
WINTYTGEPTYAADFKR

SEQ ID NO: 338 - protein, 17 aa, synthetic construct
AIYPYSGYTNYADSVKG

SEQ ID NO: 339 - protein, 17 aa, synthetic construct
AIWPFGGYTHYADSVKG

SEQ ID NO: 340 - protein, 17 aa, synthetic construct
DIAPYAGATAYADSVKG

SEQ ID NO: 341 - protein, 16 aa, synthetic construct
AIAPYSGSTYYADSVK

SEQ ID NO: 342 - protein, 12 aa, synthetic construct
WGHSTSPWAMDY

SEQ ID NO: 343 - protein, 11 aa, synthetic construct
SSYAYYAAMDY

SEQ ID NO: 344 - protein, 10 aa, synthetic construct
SYAYYSAMDY

SEQ ID NO: 345 - protein, 7 aa, synthetic construct
DVSTAVA

SEQ ID NO: 346 - protein, 6 aa, synthetic construct
SASFLY

SEQ ID NO: 347 - protein, 9 aa, synthetic construct
QQYLYHPAT

SEQ ID NO: 348 - protein, 5 aa, synthetic construct
DSWIH

SEQ ID NO: 349 - protein, 10 aa, synthetic construct
WISPYGGSTY

SEQ ID NO: 350 - protein, 7 aa, synthetic construct
RHWPGGF

SEQ ID NO: 351 - protein, 11 aa, synthetic construct
KASQDVSIAVA

SEQ ID NO: 352 - protein, 7 aa, synthetic construct
SASYRTT

SEQ ID NO: 353 - protein, 9 aa, synthetic construct
QQHYITPLT

SEQ ID NO: 354 - protein, 5 aa, synthetic construct
YNYGM

SEQ ID NO: 355 - protein, 17 aa, synthetic construct
WINTYTGEPTYTDDFKG

SEQ ID NO: 356 - protein, 12 aa, synthetic construct
GGFGSSYWYFDV

SEQ ID NO: 357 - protein, 8 aa, synthetic construct
RVSSSYLA

SEQ ID NO: 358 - protein, 7 aa, synthetic construct
SVSSYLA

SEQ ID NO: 359 - protein, 8 aa, synthetic construct
VGGYNYVS

SEQ ID NO: 360 - protein, 13 aa, synthetic construct
TRSSGSIDSNYVQ

SEQ ID NO: 361 - protein, 6 aa, synthetic construct
DASSRA

SEQ ID NO: 362 - protein, 6 aa, synthetic construct
DASNRA

SEQ ID NO: 363 - protein, 6 aa, synthetic construct
DVSNRP

SEQ ID NO: 364 - protein, 7 aa, synthetic construct
EDNQRPS

SEQ ID NO: 365 - protein, 9 aa, synthetic construct
QQYGSLPWT

SEQ ID NO: 366 - protein, 8 aa, synthetic construct
QQRSNWPT

SEQ ID NO: 367 - protein, 10 aa, synthetic construct
SSYTSSSTRV

SEQ ID NO: 368 - protein, 11 aa, synthetic construct
QSYDSNNRHVI

SEQ ID NO: 369 - protein, 5 aa, synthetic construct
RYWMS

SEQ ID NO: 370 - protein, 5 aa, synthetic construct
TYAIS

SEQ ID NO: 371 - protein, 5 aa, synthetic construct
SYIMM

SEQ ID NO: 372 - protein, 9 aa, synthetic construct
GTFSRSAIS

SEQ ID NO: 373 - protein, 10 aa, synthetic construct
NIKQDGSEKY

SEQ ID NO: 374 - protein, 10 aa, synthetic construct
GIIPIFGKAH

SEQ ID NO: 375 - protein, 10 aa, synthetic construct
SIYPSGGITF

SEQ ID NO: 376 - protein, 17 aa, synthetic construct
VIIPAFGEANYAQKFQG

SEQ ID NO: 377 - protein, 10 aa, synthetic construct
EGGWFGELAF

SEQ ID NO: 378 - protein, 12 aa, synthetic construct
KFHFVSGSPFGM

SEQ ID NO: 379 - protein, 9 aa, synthetic construct
IKLGTVTTV

SEQ ID NO: 380 - protein, 13 aa, synthetic construct
ARGRQMFGAGIDF

SEQ ID NO: 381 - protein, 11 aa, synthetic construct
RASQSISSYLN

SEQ ID NO: 382 - protein, 10 aa, synthetic construct
KASQDVSIAV

SEQ ID NO: 383 - protein, 7 aa, synthetic construct
AASSLQS

SEQ ID NO: 384 - protein, 7 aa, synthetic construct
SASYRYT

SEQ ID NO: 385 - protein, 9 aa, synthetic construct
QQSYSTPLT

SEQ ID NO: 386 - protein, 8 aa, synthetic construct
QQHYITPL

SEQ ID NO: 387 - protein, 5 aa, synthetic construct
TSYAM

SEQ ID NO: 388 - protein, 5 aa, synthetic construct
TNYGM

SEQ ID NO: 389 - protein, 17 aa, synthetic construct
WINTNTGNPTYAQGFTG

SEQ ID NO: 390 - protein, 16 aa, synthetic construct
WINTYTGEPTYTDDFK

SEQ ID NO: 391 - protein, 11 aa, synthetic construct
EDSNGYKIFDY

SEQ ID NO: 392 - protein, 11 aa, synthetic construct
GGFGSSYWYFD

SEQ ID NO: 393 - protein, 108 aa, synthetic construct
DIQMTQSPSSLSASVGDSVTITCRASQSINNYLNWYQQKPGKAPKLLIYTASSLLSGVPSRFSGSGSGTDFTLTISGLHPEDFATYFCQQSFSTPPITFGQGTRLDIK

SEQ ID NO: 394 - protein, 117 aa, synthetic construct
EVQLVESGGALVQTGGSLRLSCVASGFTFSNYWMSWVRQSPGKGLQWVASIKKDGSDEDYVDSVKGRFTISRDNAENSLYLQMTSLRIEDTAVYYCARFISAVGVDWGQGALVTVSS

SEQ ID NO: 395 - protein, 8 aa, synthetic construct
GFTFSNYW

SEQ ID NO: 396 - protein, 8 aa, synthetic construct
IKKDGSDE

SEQ ID NO: 397 - protein, 10 aa, synthetic construct
ARFISAVGVD

SEQ ID NO: 398 - protein, 107 aa, synthetic construct
DIQMTQSPSSLSASVGDRVTITCRASQGIRNDLGWYQQKPGRAPKRLIYGASNLQSGVPSRFSGSGSGTEFTLTISSLQPEDFATYYCLQHNSHPYTFGQGTKLEIK

SEQ ID NO: 399 - protein, 127 aa, synthetic construct
QVQLVESGGGVVQPGRSLRLSCAASGFTFITYGMHWVRQAPGKGLEWVAIIYYDESNKYYADSVKGRFTISRDISKNTLYLQMNGLRAEDTAVYYCARAPRVAVEEDYSYYYGMDVWGQGTTVTVSS

SEQ ID NO: 400 - protein, 20 aa, synthetic construct
ARAPRVAVEEDYSYYYGMDV

SEQ ID NO: 401 - protein, 108 aa, synthetic construct
DIVMTQSHKFMSTSVGDRVSIICKASQDVGTAVDWYQQKPGQSPKLLIYWASTRHTGVPDRFTGSGSGTDFTLAITNVQSEDLADYFCQQYNSYPLTFGAGTKLEIKR

SEQ ID NO: 402 - protein, 112 aa, synthetic construct
EVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLT

SEQ ID NO: 403 - protein, 5 aa, synthetic construct
EYTIH

SEQ ID NO: 404 - protein, 109 aa, synthetic construct
EIVLTQSPITMAAFLGERITITCSASSSISSNYLHWYQQKPGFSPKLLIYRTSNLASGVPIRFSGSGSGTSYSLTIGTMEAEDVATYYCQQGSYIPFTFGSGTKLEIKR

SEQ ID NO: 405 - protein, 119 aa, synthetic construct
QVQLQESGGGLVKPGGSLKLSCAASGFTFSDFYMYWVRQTPEKRLEWVATISDGGGYTSYPDSVKGRFTISRDIAKNNLYLQMNSLKSEDTAKYYCARGLWLRDALDYWGQGTSVTVSS

SEQ ID NO: 406 - protein, 16 aa, synthetic construct
TISDGGGYTSYPDSVK

SEQ ID NO: 407 - protein, 107 aa, synthetic construct
NIVMTQFPKSMSISVGERVTLTCKASENVGTYVSWYQQKPEQSPKMLIYGASNRFTGVPDRFTGSGSATDFILTISSVQTEDLVDYYCGQSYTFPYTFGGGTKLEMK

SEQ ID NO: 408 - protein, 116 aa, synthetic construct
EVKLEESGGGLVQPGGSMKLSCVASGFTFSNYWMNWVRQSPEKGLEWVAEIRSQSNNFATHYAESVKGRVIISRDDSKSSVYLQMNNLRAEDTGIYYCTRRWNNFWGQGTTLTVSS

SEQ ID NO: 409 - protein, 111 aa, synthetic construct
DIVLTQSPASLAVSLGQRATISCRASESIDSYDNTFMHWYQQKPGQPPNLLIFRASILESGIPARFSGSGSGTDFTLTIYPVEADDVATYYCHQSIEDPYTFGGGTKLEIK

SEQ ID NO: 410 - protein, 118 aa, synthetic construct
EVQLQQSGPELVKPGASVKMSCKASGYTFTGYVMHWVKQKPGQVLEWIGYINPYNDVTRYNGKFKGKATLTSDKYSSTAYMELSGLTSEDSAVYYCARGENWYYFDSWGRGATLTVSS

SEQ ID NO: 411 - protein, 10 aa, synthetic construct
GYTFTGYVMH

SEQ ID NO: 412 - protein, 107 aa, synthetic construct
NIVMTQSQKFMSTSPGDRVRVTCKASQNVGSDVAWYQAKPGQSPRILIYSTSYRYSGVPDRFTAYGSGTDFTLTITNVQSEDLTEYFCQQYNSYPLTFGAGTKLELK

SEQ ID NO: 413 - protein, 121 aa, synthetic construct
QVQLKESGPGLVASSQSLSITCTVSGFSLTAYGINWVRQPPGKGLEWLGVIWPDGNTDYNSTLKSRLNIFKDNSKNQVFLKMSSFQTDDTARYFCARDSYGNFKRGWFDFWGQGTTLTVSS

SEQ ID NO: 414 - protein, 16 aa, synthetic construct
VIWPDGNTDYNSTLKS

SEQ ID NO: 415 - protein, 112 aa, synthetic construct
DIVMTQSPDSLAVSLGERATINCKSSQSLLNSRNRKNYLAWYQQKPDQSPKLLIYWASTRESGVPDRFSGSGSGTDFTLTISSLQAEDVAVYYCKQSYYLWTFGGGTKVEIK

SEQ ID NO: 416 - protein, 121 aa, synthetic construct
EVQLVESGGGLVQPGGSLRLSCAASGFTFSDYGMAWFRQAPGKGLEWVSFISNLGYSIYYADSVKGRFTISRDNAKNSLYLQMNSLRAEDTAVYYCARAPYDYDSFDPMDYWGQGTLVTVS

SEQ ID NO: 417 - protein, 112 aa, synthetic construct
DIVMTQSPDSLAVSLGERATINCKSSQSLLNSRTRKNYLAWYQQKPDQSPKLLIYWASTRESGVPDRFSGSGSGTDFTLTISSLQAEDVAVYYCKQSYYLWTFGGGTKVEIK

SEQ ID NO: 418 - protein, 122 aa, synthetic construct
EVQLVESGGGLVQPGGSLRLSCAASGFTFSDYGMAWFRQAPGKGPEWVSFISNLAYSIYYADSVKGRFTISRDNAKNSLYLQMNSLRAEDTAVYYCARAPYDYDSFYPMDYWGQGTLVTVSS

SEQ ID NO: 419 - protein, 112 aa, synthetic construct
DIVMTQSPDSLAVSLGERATINCKSSQSLLNSRTRKNYLAWYQQKPDQSPKLLIYWASTRASGVPDRFSGSGSGTDFTLTISSLQAEDVAVYYCKQSYYLWTFGGGTKVEIK

SEQ ID NO: 420 - protein, 122 aa, synthetic construct
EVQLVESGGGLVQPGGSLRLSCAASGFTFSDYGMAWFRQAPGKGPEWVSFISNLAYSIYYADSVKGRFTISRDNAKNSLYLQMNSLRAEDTAVYYCARAPYDYDSFDPMDYWGQGTLVTVSS

SEQ ID NO: 421 - protein, 122 aa, synthetic construct
EVQLVESGGGLVQPGGSLRLSCAASGFTFSDVGMAWFRQAPGKGPEWVSFISNLAVSIVVADSVKGRFTISRDNAKNSLVLQMNSLRAEDTAVVVCARAPVDYDSFDPMDYWGQGTLVTVSS

SEQ ID NO: 422 - protein, 112 aa, synthetic construct
DIVMTQSPDSLAVSLGERATINCKSSQSLLNSRNRKSYLAWYQQKPDQSPKLLIYYASTRASGVPDRFSGSGSGTDFTLTISSLQAEDVAVYYCKQSYYLWTFGGGTKVEIK

SEQ ID NO: 423 - protein, 122 aa, synthetic construct
EVQLVESGGGLVQPGGSLRLSCAASGFTFSDYGMAWFRQAPGKGPEWVSFISNLAYSIYYADSVKGRFTISRDNAKNSLYLQMNSLRAEDTAVYYCARAPYDVDSFDPMDYWGQGTLVTVSS

SEQ ID NO: 424 - protein, 112 aa, synthetic construct
DIVMTQSPDSLAVSLGERATINCKSSQSLLNSRNRKSYLAWYQQKPDQSPKLLIYVASTRASGVPDRFSGSGSGTDFTLTISSLQAEDVAVYYCKQSYYLWTFGGGTKVEIK

SEQ ID NO: 425 - protein, 112 aa, synthetic construct
DVVMTQTPLSLPVSLGDQASISCRSSQSLVHSNGNTYLHWYLQRPGQSPKLLIYKVSNRFSGVPDRFSGSGSGTDFTLKISRVEAEDLGVYFCSQSTHVPFTFGSGTKLEIK

SEQ ID NO: 426 - protein, 119 aa, synthetic construct
EVKLVESGGGLVQPGGSLRLSCATSGFTFTDYHLNWVRQPPGKALEWLALIRNKRYGYRTEYSASVKGRFTISRDNSQSILYLQMNTLRAEDSATYYCARDFYDPFAYWGQGTLVTVSA

SEQ ID NO: 427 - protein, 16 aa, synthetic construct
IRNKRYGYRTEYSASV

SEQ ID NO: 428 - protein, 112 aa, synthetic construct; identical to SEQ ID NO: 425

SEQ ID NO: 429 - protein, 119 aa, synthetic construct; identical to SEQ ID NO: 426

SEQ ID NO: 430 - protein, 112 aa, synthetic construct
DVVMTQTPLSLPVSLGDQASISCRSSQSLVHSNGNTYLHWYLQRPGQSPNLLIYKVSNRFSGVPDRFSGSGSGTDFTLKISRVEAEDLGLYFCSQSTRVPFTFGSGTKLEIK

SEQ ID NO: 431 - protein, 119 aa, synthetic construct
EVKLVESGGGLVQPGGSLRLSCATSGFTFTDYHMNWVRQPPGKALEWLALIRNKANGYRTEYSASVKGRFTISRDNSQNILYLQMNTLRAEDSATYYCARDFYDPFAYWGQGTLVTVSA

SEQ ID NO: 432 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYKFPYTFGQGTKLEI

SEQ ID NO: 433 - protein, 118 aa, synthetic construct
EVQIIESGGGIVQPGGSLRLSCAASGFTFSSYAMSWVRQAPGKGIEWVSAISGSGGTTYYADSVKGRFTISRDNSKNTLYIQMNSLRAEDTAVYYCAKDAGRPYFDYWGQGTLVTVSS

SEQ ID NO: 434 - protein, 9 aa, synthetic construct
QQSYKFPYT

SEQ ID NO: 435 - protein, 8 aa, synthetic construct
DAGRPYFD

SEQ ID NO: 436 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKLEIK

SEQ ID NO: 437 - protein, 120 aa, synthetic construct
EVQLLESGGGLVQPGGSLRLSCAASGFTFSSYAMSWVRQAPGKGLEWVSAISGSGGGTRYADSVKGRFTISRDNSKNTLYLQMNSLRAEDTAVYYCAKHSSGFHWYFDYWGQGTLVTVSS

SEQ ID NO: 438 - protein, 10 aa, synthetic construct
RASQSISSYL

SEQ ID NO: 439 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSSPYTFGQGTKLEIK

SEQ ID NO: 440 - protein, 121 aa, synthetic construct
EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCAKISFYPGGTYFDYWGQGTLVTVSS

SEQ ID NO: 441 - protein, 9 aa, synthetic construct
QQSYSSPYT

SEQ ID NO: 442 - protein, 17 aa, synthetic construct
GISGSGGSTYYADSVKG

SEQ ID NO: 443 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 444 - protein, 121 aa, synthetic construct; identical to SEQ ID NO: 440

SEQ ID NO: 445 - protein, 9 aa, synthetic construct
QQSYSTPYT

SEQ ID NO: 446 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYVCQQSYSTPYTFGQGTKLEIK

SEQ ID NO: 447 - protein, 119 aa, synthetic construct
EVQLLESGGGLVQPGGSLRLSCAASGFTFSSYAMNWVRQAPGKGLEWVSTISSSGSRTYYADSVKGRFTISRDNSKNTLYLQMNSLRAEDTAVYVCAKGLVASAPFDYWGQGTLVTVSS

SEQ ID NO: 448 - protein, 17 aa, synthetic construct
TISSSGSRTYYADSVKG

SEQ ID NO: 449 - protein, 107 aa, synthetic construct; identical to SEQ ID NO: 443

SEQ ID NO: 450 - protein, 121 aa, synthetic construct
EVQLLESGGGLVQPGGSLRLSCAASGFTFSSYAMSWVRQAPGKGLEWVSGISGGGGSTYYADSVKGRFTISRDNSKNTLYLQMNSLRAEDTAVYYCAKIAHSRIGWHFDYWGQGTLVTVSS

SEQ ID NO: 451 - protein, 107 aa, synthetic construct; identical to SEQ ID NO: 443

SEQ ID NO: 452 - protein, 121 aa, synthetic construct
EVQLLESGGGLVQPGGSLRLSCAASGFTFSRYAMTWVRQAPGKGLEWVSSISASGGSTYYADSVKGRFTISRDNSKNTLYLQMNSLRAEDTAVYVCAKTFSGYAHYDFDYWGQGTLVTVSS

SEQ ID NO: 453 - protein, 17 aa, synthetic construct
SISASGGSTYYADSVKG

SEQ ID NO: 454 - protein, 108 aa, synthetic construct
EIVLTQSPGTLSLSPGERATLSCRASQSVSRNYLAWYQQKPGQAPRLLIYGASSRATGIPDRFSGSGSGTDFTLTISRLEPEDFAVYYCQQSLGYPPTFGQGTKVEIK

SEQ ID NO: 455 - protein, 116 aa, synthetic construct
EVQLLESGGGLVQPGGSLRLSCAASGFTFSPAYMSWVRQAPGKGLEWVSAISGSGGSTYYADSVKGRFTISRDNSKNTLYLQMNSLRAEDTAVYYCAKGWRAFDYWGQGTLVTVSS

SEQ ID NO: 456 - protein, 10 aa, synthetic construct
GFTFSPAYMS

SEQ ID NO: 457 - protein, 16 aa, synthetic construct
AISGSGGSTYYADSVK

SEQ ID NO: 458 - protein, 7 aa, synthetic construct
GWRAFDY

SEQ ID NO: 459 - protein, 108 aa, synthetic construct [sequence omitted]

SEQ ID NO: 460 - protein, 118 aa, synthetic construct [sequence omitted]

SEQ ID NO: 461 - protein, 18 aa, synthetic construct
VIWYDGSKKYYVDSVKGR

SEQ ID NO: 462 - protein, 118 aa, synthetic construct
QVQLVQSGGGVVQSGRSLRLSCAASGFKFSGYGMHWVRQAPGKGLEWVAVIWYDGSKKYYVDSVKGRFTISRDNSKNTLYLQMNSLRGEDTAVYYCARQMGYWHFDLWGRGTLVTVSS

SEQ ID NO: 463 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 464 - protein, 118 aa, synthetic construct
QVQLVESGGGVVQPGRSLRLSCAASGFTFRSYGMHWVRQAPGKGLEWVAIIWYDGSKKNYADSVKGRFTISRDNSKNTLYLQMNSLRAEDTAVYYCARGTGYNWFDPWGQGTLVTVSS

SEQ ID NO: 465 - protein, 109 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 466 - protein, 125 aa, synthetic construct
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 467 - protein, 14 aa, synthetic construct
GSSTGAVTSGYYPN

SEQ ID NO: 468 - protein, 7 aa, synthetic construct
GTKFLAP

SEQ ID NO: 469 - protein, 9 aa, synthetic construct
ALWYSNRWV

SEQ ID NO: 470 - protein, 5 aa, synthetic construct
IYAMN

SEQ ID NO: 471 - protein, 19 aa, synthetic construct
RIRSKYNNYATYYADSVKS

SEQ ID NO: 472 - protein, 14 aa, synthetic construct
HGNFGNSYVSFFAY

SEQ ID NO: 473 - protein, 109 aa, synthetic construct; identical to SEQ ID NO: 465

SEQ ID NO: 474 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 475 - protein, 5 aa, synthetic construct
KYAMN

SEQ ID NO: 476 - protein, 14 aa, synthetic construct
HGNFGNSYLSFWAY

SEQ ID NO: 477 - protein, 109 aa, synthetic construct; identical to SEQ ID NO: 465

SEQ ID NO: 478 - protein, 125 aa, synthetic construct
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 479 - protein, 5 aa, synthetic construct
RYAMN

SEQ ID NO: 480 - protein, 16 aa, synthetic construct
RIRSKYNNYATYYADS

SEQ ID NO: 481 - protein, 14 aa, synthetic construct
HGNFGNSYLSYFAY

SEQ ID NO: 482 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 483 - protein, 123 aa, synthetic construct
AVQLVESGGGLVQPGRSLRLSCAASGFTFGDYTMHWVRQAPGKGLEWVSGISWNSGSIGYADSVKGRFTISRDNAKNSLYLQMNSLRAEDTALYYCTKDNQYGSGSTYGLGVWGQGTLVTVSS

SEQ ID NO: 484 - protein, 9 aa, synthetic construct
QQRSNWPLT

SEQ ID NO: 485 - protein, 5 aa, synthetic construct
DYTMH

SEQ ID NO: 486 - protein, 18 aa, synthetic construct
GISWNSGSILGYADSVKG

SEQ ID NO: 487 - protein, 14 aa, synthetic construct
DNQYGSGSTYGLGV

SEQ ID NO: 488 - protein, 106 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSVFTFGPGTKVDIK

SEQ ID NO: 489 - protein, 123 aa, synthetic construct; identical to SEQ ID NO: 483

SEQ ID NO: 490 - protein, 11 aa, synthetic construct
RASQGISSWLA

SEQ ID NO: 491 - protein, 8 aa, synthetic construct
QQYNSVFT

SEQ ID NO: 492 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIK

SEQ ID NO: 493 - protein, 123 aa, synthetic construct; identical to SEQ ID NO: 483

SEQ ID NO: 494 - protein, 9 aa, synthetic construct
QQRSDWPLT

SEQ ID NO: 495 - protein, 106 aa, synthetic construct
QIVLSQSPAILSASPYEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 496 - protein, 119 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGTTVTVSA

SEQ ID NO: 497 - protein, 112 aa, synthetic construct
DIVMTQSPDSLAVSLGERATINCKSSQSLFNSRTRKNYLAWYQQKPGQPPKLLIYWASTRESGVPDRFSGSGSGTDFTLTISSLQAEDVAVYYCMQSFNLRTFGQGTKVEIK

SEQ ID NO: 498 - protein, 120 aa, synthetic construct
EVQLVESGGGLVQPGRSLRLSCTASGFTFTDNYMSWVRQAPGKGLEWVGFIRNKANGYTTEYAASVKGRFTISRDDSKSIAYLQMNSLKTEDTAVYYCARDVGSNYFDYWGQGTLVTVSS

SEQ ID NO: 499 - protein, 12 aa, synthetic construct
NSLFNSRTRKNY

SEQ ID NO: 500 - protein, 112 aa, synthetic construct
DIVMTQSPSSLAVSLGERATMSCKSSQSLFNSRTRKNYLAWYQQKPGQSPKLLIYWASARDSGVPARFTGSGSETYFTLTISRVQAEDLAVYYCMQSFNLRTFGQGTKVEIK

SEQ ID NO: 501 - protein, 120 aa, synthetic construct
EVNLVESGGGLVQPGRSLRLSCTASGFTFTDNYMSWVRQAPGKGLEWLGFIRNKANGYTTDYAASVRGRFTISRDNSKSILYLQMNALRTEDTAVYYCARDVGSNYFDYWGQGTLVTVSS

SEQ ID NO: 502 - protein, 112 aa, synthetic construct
DIVMSQSPSSLAVSAGEKVTMSCKSSQSLFNSRTRKNYLAWYQQKPGQSPKLLIYWASARDSGVPARFTGSGSETYFTLTISRVQAEDLAVYYCMQSFNLRTFGGGTKLEIK

SEQ ID NO: 503 - protein, 120 aa, synthetic construct
EVNLVESGGGLVQPGGSLRLSCATSGFTFTDNYMSWVRQPPGKALEWLGFIRNKANGYTTDYSASVRGRFTISRDNSQSILYLQMNALRAEDSATYYCARDVGSNYFDYWGQGTTLTVSS

SEQ ID NO: 504 - protein, 12 aa, synthetic construct
QSLFNSRTRKNY

SEQ ID NO: 505 - protein, 109 aa, synthetic construct
SSELTQDPAVSVALGQTVRITCQGDSLRKFFASWYQQKPGQAPVLVIYGKNSRPSGIPDRFSGSNSRNTASLTITGAQAEDEGDYYCNSRDSRDNHQVFGAGTKVTVLS

SEQ ID NO: 506 - protein, 126 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDTPGIAARRYYYYGMDVWGQGTTVTVSS

SEQ ID NO: 507 - protein, 111 aa, synthetic construct
QPVLTQPRSVSGSPGQSVTISCTGTISDVGGHNFVSWYQQNPGKAPKLIIFEVTKRPAGVPDRFSGSKSGNTASLTVSGLQAEDEGEYYCSSYGGSNDVIFGGGTKLSVLG

SEQ ID NO: 508 - protein, 121 aa, synthetic construct
EVQLVESGGGVVQPGRSLRLSCAASGFTFSSYGMHWVRQAPGKGLEWVAVISYDGSNKYYADSVKGRFTISRDNSKNTLYLQVSSLRAEDTAVYYCVRDLVAAAGTAFDIWGQGTTVTVSS

SEQ ID NO: 509 - protein, 111 aa, synthetic construct
QSVLTQPPSVSKGLRQTATLTCTGNSNNVGNQGAAWLQQHQGHPPKLLSYRNNNRPSGISERFSASRSGNTASLTITGLQPEDEADYYCSAWDNRLKTYVFGTGGKVTVLG

SEQ ID NO: 510 - protein, 120 aa, synthetic construct
QVQLQQSGPGLVKPSQTLSLTCAVSGDSVSSNFVAWNWIRQSPSRGLEWLGRTYYRSRWYNDYAVSVQSRIRVTPDTSKNQFSLHLDSVTPEDTAVYYCARGQHSGFDFWGQGTLVTVSS

SEQ ID NO: 511 - protein, 108 aa, synthetic construct
EIVLTQSPATLSVSPGERATLSCRASQSVNTNLAWYQQKPGQAPRLLIYGASSRATGIPDRFSGSGSGTDFTLTISRLEPEDFAVYYCQHYGSSPLTFGGGTKLEIKR

SEQ ID NO: 512 - protein, 121 aa, synthetic construct
QVQLVQSGGGVVQPGRSLRLSCAASGFTFSSYPMHWVRQAPGKGLEWMTVISSDGRNKYYPDSVKGRFTISRDNSKNTLYLQMNSLRPEDTAVYYCARGGYHDFWSGPDYWGQGTLVTVSS

SEQ ID NO: 513 - protein, 107 aa, synthetic construct
SSELTQDPAVSVALGQTVRITCQGDSLRSYYASWYQQKPGQAPVLVIYGKNNRPSGIPDRFSGSKSHNTAYLTITGAQAEDEADYFCNSRSGSQRVFGGGTKLTVLG

SEQ ID NO: 514-523 - skipped (10 entries)

SEQ ID NO: 524 - protein, 127 aa, synthetic construct
EVQLVQSGGGLVQPGGSLRLSCAASGFTFSSYAMSWVRQAPGKGLEWVANIKQDGSEKYYVDSVKGRFTISRDNAKNSLYLQMNSLRAEDTAVYYCARDQVSGITIFGGKWRSPDVWGQGTTVTVSS

SEQ ID NO: 525 - protein, 111 aa, synthetic construct
QSVLTQPPSASGTPGQRVTISCSGSRSNIGSNTVNWYQQLPGTAPKLLIYTNNQRPSGVPDRFSGSKSGTSASLAITGLQAEDEADYYCLSFDSSLTSYVFGTGTKVTVLG

SEQ ID NO: 526 - protein, 126 aa, synthetic construct
QVTLKESGGGVVQPGRSLRLSCAASGFTFTNYGLHWVRQAPGKGLEWVAVISHDGTKKYYADSVKGRFTISRDSSENTLYLQMNSLRPEDSALYYCARDGGYCSGGRCYSYGMDVWGQGTTVTVSS

SEQ ID NO: 527 - protein, 112 aa, synthetic construct
QSVLTQPPSASGTPGQRVTISCSGSRSNIGGNTVNWYQQLPGTAPKLLIYANNQRPSGVPDRFSGSKSGTSASLAISGLRSEDEADYYCAAWDDNLSGHVVFGGGTKLTVLR

SEQ ID NO: 528 - protein, 122 aa, synthetic construct
QVQLVQSGGGVVLPGRSLRLSCVASGFTFRRYGMHWVRQAPGKGLEWVSLISYDGSKTFYGESVKGRFTISRDNSKNTLYLQMNSLRAEDTAVYYCARATVTTDGYYYMDVWGKGTTVTVSS

SEQ ID NO: 529 - protein, 12 aa, synthetic construct
AAWDDNLSGHVV

SEQ ID NO: 530 - protein, 108 aa, synthetic construct
DIQMTQSPSSLSASVGDRVTITCRASQDVNTAVAYQQKPGKAPKLLIYSASFLYSGVPSRFSGSRSGTDFTLTISSLQPEDFATYYCQQHYTTPPTFGQGTKVEIKRT

SEQ ID NO: 531 - protein, 121 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSA

SEQ ID NO: 532 - protein, 9 aa, synthetic construct
QQHYTTPPT

SEQ ID NO: 533 - protein, 17 aa, synthetic construct
RIYPTNGYTRYADSVKG

SEQ ID NO: 534 - protein, 107 aa, synthetic construct [sequence omitted]

SEQ ID NO: 535 - protein, 119 aa, synthetic construct [sequence omitted]

SEQ ID NO: 536 - protein, 6 aa, synthetic construct
SASYRY

SEQ ID NO: 537 - protein, 8 aa, synthetic construct
QQYYIYPY

SEQ ID NO: 538 - protein, 17 aa, synthetic construct
ADVNPNSGGSIYNQRFK

SEQ ID NO: 539 - protein, 12 aa, synthetic construct
ARNLGPSFYFDY

SEQ ID NO: 540 - protein, 107 aa, synthetic construct
DTVMTQSHKIMSTSVGDRVSITCKASQDVSIGVAWYQQRPGQSPKLLIYSASYRYTGVPDRFTGSGSGTDFTFTISSVQAEDLAVYYCQQYYIYPYTFGGGTKLEIK

SEQ ID NO: 541 - protein, 119 aa, synthetic construct
EVQLQQSGPELVKPGTSVKISCKASGFTFTDYTMDWVKQSHGKSLEWIGDVNPNSGGSIYNQRFKGKASLTVDRSSRIVYMELRSLTFEDTAVYYCARNLGPSFYFDYWGQGTTLTVSS

SEQ ID NO: 542 - protein, 107 aa, synthetic construct [sequence omitted]

SEQ ID NO: 543 - protein, 119 aa, synthetic construct [sequence omitted]

SEQ ID NO: 544 - protein, 107 aa, synthetic construct
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIK

SEQ ID NO: 545 - protein, 119 aa, synthetic construct
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 546 - protein, 11 aa, synthetic construct
RASQSISNYLA

SEQ ID NO: 547 - protein, 7 aa, synthetic construct
AASSLES

SEQ ID NO: 548 - protein, 9 aa, synthetic construct
QQYNSLPWT

SEQ ID NO: 549 - protein, 10 aa, synthetic construct
GFTFSSYAMS

SEQ ID NO: 550 - protein, 17 aa, synthetic construct
VISGDGGSTYYADSVKG

SEQ ID NO: 551 - protein, 10 aa, synthetic construct
GRVGYSLYDY

SEQ ID NO: 552 - protein, 107 aa, synthetic construct [sequence omitted]

SEQ ID NO: 553 - protein, 122 aa, synthetic construct
EVQLVESGGGLVQPGGSLRLSCAASGYTFTNYGMNWVRQAPGKGLEWVGWINTYTGEPTYAADFKRRFTSLDTSKSTAYLQMNSLRAEDTAVYYCAKYPHYYGSSHWYFDVWGQGTLVTVSS

SEQ ID NO: 554 - protein, 6 aa, synthetic construct
QDISNY

SEQ ID NO: 555 - skipped

SEQ ID NO: 556 - protein, 8 aa, synthetic construct
GYTFTNYG

SEQ ID NO: 557 - protein, 8 aa, synthetic construct
INTYTGEP

SEQ ID NO: 558 - protein, 16 aa, synthetic construct
AKYPHYYGSSHWYFDV

SEQ ID NO: 559 - protein, 107 aa, synthetic construct [sequence omitted]

SEQ ID NO: 560 - protein, 122 aa, synthetic construct
EVQLVESGGGLVQPGGSLRLSCAASGYDFTHYGMNWVRQAPGKGLEWVGWINTYTGEPTYAADFKRRFTSLDTSKSTAYLQMNSLRAEDTAVYYCAKYPYYYGTSHWYFDVWGQGTLVTVSS

SEQ ID NO: 561 - protein, 8 aa, synthetic construct
GYDFTHYG

SEQ ID NO: 562 - protein, 16 aa, synthetic construct
AKYPYYYGTSHWYFDV

SEQ ID NO: 563 - protein, 111 aa, synthetic construct
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLG

SEQ ID NO: 564 - protein, 104 aa, synthetic construct
SLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 565 - protein, 11 aa, synthetic construct
QASEIIHSWLA

SEQ ID NO: 566 - protein, 7 aa, synthetic construct
LASTLAS

SEQ ID NO: 567 - protein, 12 aa, synthetic construct
QNVYLASTNGAN

SEQ ID NO: 568 - protein, 11 aa, synthetic construct
GFSLTDYYYMT

SEQ ID NO: 569 - protein, 16 aa, synthetic construct
FIDPDDDPYYATWAKG

SEQ ID NO: 570 - protein, 11 aa, synthetic construct
GDHNSGWGLDI

SEQ ID NO: 571 - protein, 112 aa, synthetic construct
EIVMTQSPSTLSASVGDRVIITCQSSQSVYGNIWMAWYQQKPGKAPKLLIYQASKLASGVPSRFSGSGSGAEFTLTISSLQPDDFATYYCQGNFNTGDRYAFGQGTKLTVLG

SEQ ID NO: 572 - protein, 121 aa, synthetic construct
EVQLVESGGGLVQPGGSLRLSCAASGFTISRSYWICWVRQAPGKGLEWVSCIYGDNDITPLYANWAKGRFTISRDNSKNTLYLQMNSLRAEDTAVYYCAKLGYADYAYDLWGQGTLVTVSS

SEQ ID NO: 573 - protein, 13 aa, synthetic construct
QSSQSVYGNIWMA

SEQ ID NO: 574 - protein, 7 aa, synthetic construct
QASKLAS

SEQ ID NO: 575 - protein, 11 aa, synthetic construct
QGNFNTGDRYA

SEQ ID NO: 576 - protein, 11 aa, synthetic construct
GFTISRSYWIC

SEQ ID NO: 577 - protein, 16 aa, synthetic construct
CIYGDNDITPLYANWA

SEQ ID NO: 578 - protein, 10 aa, synthetic construct
LGYADYAYDL

SEQ ID NO: 579 - protein, 110 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTV

SEQ ID NO: 580 - protein, 118 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTL

SEQ ID NO: 581 - protein, 14 aa, synthetic construct
YPHYYGSSHWYFDV

SEQ ID NO: 582 - protein, 110 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTV

SEQ ID NO: 583 - protein, 118 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTL

SEQ ID NO: 584 - protein, 14 aa, synthetic construct
YPYYYGTSHWYFDV

SEQ ID NO: 585 - protein, 107 aa, synthetic construct [sequence omitted]

SEQ ID NO: 586 - protein, 115 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVT

SEQ ID NO: 587 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 588 - protein, 118 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVT

SEQ ID NO: 589 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 590 - protein, 120 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVT

SEQ ID NO: 591 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 592 - protein, 117 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVT

SEQ ID NO: 593 - protein, 120 aa, synthetic construct
QVQLVQSGAEVKKPGSSVKVSCKASGGTFSRSAISWVRQAPGQGLEWMGVIIPAFGEANYAQKFQGRVTITADESTSTAYMELSSLRSEDTAVYYCARGRQMFGAGIDFWGQGTLVTVSS

SEQ ID NO: 594 - protein, 115 aa, synthetic construct; identical to SEQ ID NO: 586

SEQ ID NO: 595 - protein, 108 aa, synthetic construct
DIQLTQSHKFMSTSVGDRVSITCKASQDVSIAVAWYQQKPGQSPKLLIYSASYRYTGVPDRFTGSGSGTDFTFTISSVQAEDLAVYYCQQHYITPLTFGAGTKLELKR

SEQ ID NO: 596 - protein, 120 aa, synthetic construct
VKLQESGPELKKPGETVKISCKASGYTFTNYGMNWVKQAPGKGLKWMGWINTYTGEPTYTDDFKGRFAFSLETSATTAYLQINNLKSEDMATYFCARGGFGSSYWYFDVWGQGTTVTVSS

SEQ ID NO: 597 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEI

SEQ ID NO: 598 - protein, 108 aa, synthetic construct
VQLVQSGSELKKPGASVKVSCKASGYTFTSYAMNWVRQAPGQGLEWMGWINTNTGNPTYAQGFTGRFVFSLDTSVSTAYLQISSLKADDTAVYYCAREDSNGYKIFDY

SEQ ID NO: 599 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 600 - protein, 109 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFV

SEQ ID NO: 601 - DNA, 68 bp, synthetic construct
  source 1..68: mol_type = other DNA
gggaggacgatgcggaccgaaaaagacctgacttctatactaagtctacgttcccagacgactcccga

SEQ ID NO: 602 - DNA, 68 bp, synthetic construct
  source 1..68: mol_type = other DNA
gggaggacgatgcggatcagccatgtttacgtcactccttgtcaatcctcatcggcagactcgcccga

SEQ ID NO: 603 - RNA, 56 nt, synthetic construct
  source 1..56: mol_type = other RNA
gggaggacgatgcggatcagccatgtttacgtcactccttgtcaatcctcatcggc

SEQ ID NO: 604 - RNA, 39 nt, synthetic construct
  source 1..39: mol_type = other RNA
gggaggacgatgcggatcagccatgtttacgtcactcct

SEQ ID NO: 605 - DNA, 66 bp, synthetic construct
  source 1..66: mol_type = other DNA
agcgtcgaataccactacacaccacatccgtcttactccccaattacagaccacgagctccattag

SEQ ID NO: 606 - DNA, 66 bp, synthetic construct
  source 1..66: mol_type = other DNA
agcgtcgaataccactactccacctttccgtctaactccccactttatgaccacgagctccattag

SEQ ID NO: 607 - DNA, 66 bp, synthetic construct
  source 1..66: mol_type = other DNA
agcgtcgaataccactacccaacctttaccgtcaaactccccacttttgaccacgagctccattag

SEQ ID NO: 608 - DNA, 66 bp, synthetic construct
  source 1..66: mol_type = other DNA
agcgtcgaataccactactaagacgcaattcgaccttcttccccattcgaccacgagctccattag

SEQ ID NO: 609 - DNA, 66 bp, synthetic construct
  source 1..66: mol_type = other DNA
agcgtcgaataccactacacttacaccaactctatcatctcccttatagaccacgagctccattag

SEQ ID NO: 610 - DNA, 66 bp, synthetic construct
  source 1..66: mol_type = other DNA
agcgtcgaataccactacttacatccctacgaacaagcggactgcagagaccacgagctccattag

SEQ ID NO: 611 - DNA, 65 bp, synthetic construct
  source 1..65: mol_type = other DNA
agcgtcgaataccactacccgccatccgtcaacgtcttcccactattgaccacgagctccattag

SEQ ID NO: 612 - RNA, 48 nt, synthetic construct
  source 1..48: mol_type = other RNA
cagcgaaagttgcgtatgggtcactcgcaggcacatgtcatctgggcg

SEQ ID NO: 613 - DNA, 66 bp, synthetic construct; identical to SEQ ID NO: 605
  source 1..66: mol_type = other DNA

SEQ ID NO: 614 - DNA, 66 bp, synthetic construct; identical to SEQ ID NO: 606
  source 1..66: mol_type = other DNA

SEQ ID NO: 615 - DNA, 66 bp, synthetic construct; identical to SEQ ID NO: 607
  source 1..66: mol_type = other DNA

SEQ ID NO: 616 - DNA, 66 bp, synthetic construct; identical to SEQ ID NO: 608
  source 1..66: mol_type = other DNA

SEQ ID NO: 617 - DNA, 66 bp, synthetic construct; identical to SEQ ID NO: 609
  source 1..66: mol_type = other DNA

SEQ ID NO: 618 - DNA, 66 bp, synthetic construct; identical to SEQ ID NO: 610
  source 1..66: mol_type = other DNA

SEQ ID NO: 619 - DNA, 65 bp, synthetic construct; identical to SEQ ID NO: 611
  source 1..65: mol_type = other DNA

SEQ ID NO: 620 - RNA, 48 nt, synthetic construct; identical to SEQ ID NO: 612
  source 1..48: mol_type = other RNA

SEQ ID NO: 621 - DNA, 62 bp, synthetic construct
  source 1..62: mol_type = other DNA
acagcatccccatgtgaacaatcgcattgtgattgttacggtttccgcctcatggacgtgct

SEQ ID NO: 622 - DNA, 43 bp, synthetic construct
  source 1..43: mol_type = other DNA
ggaggcaacggagcggagacattgactgagtgaacgtgtagtg

SEQ ID NO: 623 - DNA, 25 bp, synthetic construct
  source 1..25: mol_type = other DNA
gctactgtacatcacgcctctcccc

SEQ ID NO: 624 - DNA, 24 bp, synthetic construct
  source 1..24: mol_type = other DNA
ctactgtacatcacgcctctcccc

SEQ ID NO: 625 - DNA, 25 bp, synthetic construct
  source 1..25: mol_type = other DNA
gtacagttcccgtccctgcactaca

SEQ ID NO: 626 - DNA, 24 bp, synthetic construct
  source 1..24: mol_type = other DNA
gtacagttcccgtcctgcactaca

SEQ ID NO: 627 - DNA, 45 bp, synthetic construct
  source 1..45: mol_type = other DNA
acgggccacatcaactcattgatagacaatgcgtccactgcccgt

SEQ ID NO: 628 - DNA, 25 bp, synthetic construct
  source 1..25: mol_type = other DNA
tgtgggggtggacgggccgggtaga

SEQ ID NO: 629 - DNA, 25 bp, synthetic construct
  source 1..25: mol_type = other DNA
tgtgggggaggacgggcggggatgc

SEQ ID NO: 630 - DNA, 25 bp, synthetic construct
  source 1..25: mol_type = other DNA
tgtgggggtggactgggtgggtacc

SEQ ID NO: 631 - DNA, 25 bp, synthetic construct
  source 1..25: mol_type = other DNA
tgtgggggtggacgggccgagtacg

SEQ ID NO: 632 - DNA, 25 bp, synthetic construct
  source 1..25: mol_type = other DNA
tgtggggatggatgggccgggctgc

SEQ ID NO: 633 - DNA, 25 bp, synthetic construct
  source 1..25: mol_type = other DNA
tgtgggggtggttgggcggggctgc

SEQ ID NO: 634 - DNA, 26 bp, synthetic construct
  source 1..26: mol_type = other DNA
ggtggtggtggttgtggtggtggtgg

SEQ ID NO: 635 - DNA, 29 bp, synthetic construct
  source 1..29: mol_type = other DNA
tttggtggtggtggttgtggtggtggtgg

SEQ ID NO: 636 - DNA, 25 bp, synthetic construct
  source 1..25: mol_type = other DNA
gcagttgatcctttggataccctgg

SEQ ID NO: 637 - DNA, 25 bp, synthetic construct
  source 1..25: mol_type = other DNA
ggcgtacggtaggcggggtcaactg

SEQ ID NO: 638 - DNA, 25 bp, synthetic construct
  source 1..25: mol_type = other DNA
gaagtgaaaatgacagaacacaaca

SEQ ID NO: 639 - DNA, 25 bp, synthetic construct
  source 1..25: mol_type = other DNA
ggctatagcacatgggtaaaacgag

SEQ ID NO: 640 - DNA, 25 bp, synthetic construct
  source 1..25: mol_type = other DNA
caaacaatcaaacagcagtggggtg

SEQ ID NO: 641 - DNA, 25 bp, synthetic construct
  source 1..25: mol_type = other DNA
tactgcatgcacaccacttcaacta

SEQ ID NO: 642 - DNA, 25 bp, synthetic construct
  source 1..25: mol_type = other DNA
gggttatattactcggccggtgtaa

SEQ ID NO: 643 - DNA, 25 bp, synthetic construct
  source 1..25: mol_type = other DNA
ggctatagcacatgggtaaaacgac

SEQ ID NO: 644 - DNA, 25 bp, synthetic construct
  source 1..25: mol_type = other DNA
ggcgtacggtaggcggggtcaactg

SEQ ID NO: 645 - DNA, 25 bp, synthetic construct
  source 1..25: mol_type = other DNA
gctgggtaaatagatgattcccggc

SEQ ID NO: 646 - DNA, 25 bp, synthetic construct
  source 1..25: mol_type = other DNA
agtggtggttttataagactgatac

SEQ ID NO: 647 - DNA, 25 bp, synthetic construct
  source 1..25: mol_type = other DNA
tagccttaatgcctttgtaaggcga

SEQ ID NO: 648 - DNA, 25 bp, synthetic construct
  source 1..25: mol_type = other DNA
cggtctacggagccgcgttaaactt

SEQ ID NO: 649 - DNA, 45 bp, synthetic construct
  source 1..45: mol_type = other DNA
acaccaaaatcgtccgtttcgttttagtccgtctctttagggtgt

SEQ ID NO: 650 - DNA, 51 bp, synthetic construct
  source 1..51: mol_type = other DNA
agcagcgtggaggatatgatctgaccgttttcgtttgttatagacgctgct

SEQ ID NO: 651 - DNA, 88 bp, synthetic construct
  source 1..88: mol_type = other DNA
ataccagcttattcaattagtcacacttagagttctaactgctgcgccgccgggaaaatactgtacggttagatagtaagtgcaatct

SEQ ID NO: 652 - DNA, 41 bp, synthetic construct
  source 1..41: mol_type = other DNA
atctaactgctgcgccgccgggaaaatactgtacggttaga

SEQ ID NO: 653 - DNA, 35 bp, synthetic construct
  source 1..35: mol_type = other DNA
atctaactgctgcgccgccgggnngtacggttaga

SEQ ID NO: 654 - DNA, 30 bp, synthetic construct
  source 1..30: mol_type = other DNA
tgctgttgtcacctgcctcgtctccctcgt

SEQ ID NO: 655 - DNA, 30 bp, synthetic construct
  source 1..30: mol_type = other DNA
ttccctcctctgttgttcccccctcctctc

SEQ ID NO: 656 - DNA, 30 bp, synthetic construct
  source 1..30: mol_type = other DNA
tggggtggtggtgtgggtgggggtttgttc

SEQ ID NO: 657 - DNA, 100 bp, synthetic construct
  source 1..100: mol_type = other DNA
ctcctctgactgtaaccacgccgtatgtccgaaatacggagaacagcactcatatgcaagccatacgcggaggtgcacgcgcataggtagtccagaagcc

SEQ ID NO: 658 - DNA, 69 bp, synthetic construct
  source 1..69: mol_type = other DNA
ggctgttgtgagcctcctcccagagggaagactttaggttcggttcacgtcccgcttattcttactccc

SEQ ID NO: 659 - DNA, 34 bp, synthetic construct
  source 1..34: mol_type = other DNA
cctgcacttggcttggatttcagaagggagaccc

SEQ ID NO: 660 - DNA, 59 bp, synthetic construct
  source 1..59: mol_type = other DNA
tgcgtgtgtagtgtgtctgttgtttgtattgttgtctatcctcttagggatttgggcgg

SEQ ID NO: 661 - DNA, 52 bp, synthetic construct
  source 1..52: mol_type = other DNA
acgagycyggyccyyccyyygacgacyayygyyyggyaycgaycaacaacaa

SEQ ID NO: 662 - DNA, 51 bp, synthetic construct
  source 1..51: mol_type = other DNA
acgagycacacgyygaygacyggayggyagyyaaagagggyggggcaacaa

SEQ ID NO: 663 - DNA, 51 bp, synthetic construct
  source 1..51: mol_type = other DNA
acgagyygycgcaygygcggyycagycyggyccyycagcaccgyacaacaa

SEQ ID NO: 664 - DNA, 51 bp, synthetic construct
  source 1..51: mol_type = other DNA
acgagycyggyccyyccycccyaayygcygyygaggyaycggcyacaacaa

SEQ ID NO: 665 - DNA, 37 bp, synthetic construct
  source 1..37: mol_type = other DNA
tgggagggcgcgttcttcgtggttacttttagtcccg

SEQ ID NO: 666 - DNA, 39 bp, synthetic construct
  source 1..39: mol_type = other DNA
cacaggctacggcacgtagagcatcaccatgatcctgtg

SEQ ID NO: 667 - DNA, 44 bp, synthetic construct
  source 1..44: mol_type = other DNA
tactcagggcactgcaagcaattgtggtcccaatgggctgagta

SEQ ID NO: 668 - DNA, 40 bp, synthetic construct
  source 1..40: mol_type = other DNA
cactgaacgaatggcaaccgccaaaccctacccacactgg

SEQ ID NO: 669 - protein, 9 aa, synthetic construct
KTLLPTPYC

SEQ ID NO: 670-671 - skipped (2 entries)

SEQ ID NO: 672 - protein, 13 aa, synthetic construct
CGGGPKKKRKVGG

SEQ ID NO: 673 - protein, 8 aa, synthetic construct
CRQAGFSL

SEQ ID NO: 674 - protein, 15 aa, synthetic construct
CPNFDWDPNNSNAGF

SEQ ID NO: 675 - protein, 12 aa, synthetic construct
TSFAEYWALLSP

SEQ ID NO: 676 - protein, 14 aa, synthetic construct
YQRLGNQWAVGHLM

SEQ ID NO: 677 - protein, 12 aa, synthetic construct
HTMYYHHYQHHL

SEQ ID NO: 678 - protein, 21 aa, synthetic construct
LGASWHRPDKCCLGYQKRPLP

SEQ ID NO: 679 - protein, 10 aa, synthetic construct
NAACAWLEAQ

SEQ ID NO: 680 - protein, 7 aa, synthetic construct
RACRFFC

SEQ ID NO: 681 - protein, 6 aa, synthetic construct
  SITE 3: note = D-Ornithine
  MOD_RES 3: note = N-methylated
  SITE 5: note = D-alanine
DYXRXG

SEQ ID NO: 682 - protein, 22 aa, synthetic construct
GGRSFFLLRRIQGCRFRNTVDD

SEQ ID NO: 683 - protein, 9 aa, synthetic construct
CAGRRSAYC

SEQ ID NO: 684 - protein, 9 aa, synthetic construct
CREAGRKAC

SEQ ID NO: 685 - protein, 9 aa, synthetic construct
CLSDGKRKC

SEQ ID NO: 686 - skipped

SEQ ID NO: 687 - protein, 12 aa, synthetic construct
FHKHKSPALSPV

SEQ ID NO: 688 - protein, 11 aa, synthetic construct
ACSSSPSKHCG

SEQ ID NO: 689 - protein, 7 aa, synthetic construct
CAGALCY

SEQ ID NO: 690 - protein, 29 aa, synthetic construct
YTIWMPENPRPGTPCDIFTNSRGKRASNG

SEQ ID NO: 691 - protein, 6 aa, synthetic construct
KCCYSL

SEQ ID NO: 692 - protein, 12 aa, synthetic construct
FCGDFYACYMDV

SEQ ID NO: 693 - protein, 8 aa, synthetic construct
YLFFVFER

SEQ ID NO: 694 - protein, 17 aa, synthetic construct
CDTFPYLGWWNPNEYRY

SEQ ID NO: 695 - protein, 17 aa, synthetic construct
CKTIYYLGYYNPNEYRY

SEQ ID NO: 696 - protein, 7 aa, synthetic construct
LTVSPWY

SEQ ID NO: 697 - protein, 8 aa, synthetic construct
KLRLEWNR

SEQ ID NO: 698 - protein, 9 aa, synthetic construct
QDVNTAVAW

SEQ ID NO: 699 - protein, 11 aa, synthetic construct
CGGGLTVSPWY

SEQ ID NO: 700 - protein, 16 aa, synthetic construct
MEGPSKCCYSLALASH

SEQ ID NO: 701 - protein, 14 aa, synthetic construct
GTKSKCCYSLRRSS

SEQ ID NO: 702 - protein, 11 aa, synthetic construct
CSSSLTVSPWY

SEQ ID NO: 703 - protein, 17 aa, synthetic construct
CKTIYYLGYYNPNEYRY

SEQ ID NO: 704 - protein, 19 aa, synthetic construct
GGCAKIFGSLAFLKCCYSL

SEQ ID NO: 705 - protein, 12 aa, synthetic construct
YHWYGYTPQNVI

SEQ ID NO: 706 - protein, 11 aa, synthetic construct
KASQDVSTAVA

SEQ ID NO: 707 - protein, 108 aa, synthetic construct [sequence omitted]

SEQ ID NO: 708 - protein, 16 aa, synthetic construct
IWYDGSKKYYVDSVKG

SEQ ID NO: 709 - protein, 125 aa, synthetic construct
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 710 - protein, 19 aa, synthetic construct
RIRSKYNNYATYYADSVKG

SEQ ID NO: 711 - RNA, 28 nt, synthetic construct
  source 1..28: mol_type = other RNA
cggaatcagtgaatgcttatacatccgt

SEQ ID NO: 712 - RNA, 85 nt, synthetic construct
  source 1..85: mol_type = other RNA
gggagacaagaataaacgctcaattcaacgctgtgaagggcttatacgagcggattacccttcgacaggaggctcacaaacaggc